(19) United States
(12) Patent Application Publication
(10) Pub. No.: US 2019/0359678 A1
(43) Pub. Date: Nov. 28, 2019

(54) CHIMERIC T CELL ANTIGEN RECEPTORS AND METHODS OF USE THEREOF

(71) Applicant: The Regents of the University of California, Oakland, CA (US)

(72) Inventors: Geoffrey P. O'Donoghue, San Francisco, CA (US); Jasper Z. Williams, San Francisco, CA (US); Wendell A. Lim, San Francisco, CA (US)

(21) Appl. No.: 16/483,349

(22) PCT Filed: Feb. 8, 2018
(86) PCT No.: PCT/US18/17485

§ 371 Date: Aug. 2, 2019

Related U.S. Application Data
(60) Provisional application No. 62/457,112, filed on Feb. 9, 2017.

(51) Int. Cl. C07K 14/725; A61K 35/17; C07K 16/30; A61P 35/00; C07K 16/28

(52) U.S. Cl. CPC C07K 14/7051; A61K 35/17; C07K 16/30; C07K 2319/33; C07K 16/2803; C07K 2319/03; C07K 2317/622; A61P 35/00

(57) ABSTRACT

Provided are chimeric T cell antigen receptors (TCR) comprising modified TCR chains. The modified TCR chains include fusion polypeptides having one or more heterologous antigen-binding domains fused to the extracellular domain of the TCR chain. Modified TCR chains also include chains that are modified in various other ways including e.g., chain truncation, cysteine modification, domain swapping and combinations thereof. Also provided are nucleic acids encoding the modified TCR chains as well as nucleic acids encoding the chimeric TCRs and recombinant expression vectors comprising such nucleic acids. Immune cells that are genetically modified or otherwise include the described chimeric TCRs, recombinant expression vectors encoding chimeric TCRs, and/or the described nucleic acids are also provided. Methods are also provided, such as methods of killing a target cell and/or treating a subject for a condition, e.g., through the use of the described chimeric TCRs, nucleic acids, expression vectors and/or immune cells.

### CROSS-REFERENCE

This application claims the benefit of U.S. Provisional Patent Application No. 62/457,112, filed Feb. 9, 2017, which application is incorporated herein by reference in its entirety.

### STATEMENT REGARDING FEDERALLY SPONSORED RESEARCH

This invention was made with government support under grant no. R01 CA196277 awarded by the National Institutes of Health. The government has certain rights in the invention

### INCORPORATION BY REFERENCE OF SEQUENCE LISTING PROVIDED AS A TEXT FILE

A Sequence Listing is provided herewith as a text file, “UCSF-550WO_SeqList_ST25.txt” created on Jan. 31, 2018 and having a size of 434 KB. The contents of the text file are incorporated by reference herein in their entirety.

### INTRODUCTION

Immunotherapy has rapidly advanced as an effective modality for the treatment of cancer, supplementing historical pillars of cancer treatment, namely surgery, chemotherapy, and radiotherapy. Recombinant designer immune molecules such as engineered T cell receptors (TCRs) and chimeric antigen receptors (CARs) have greatly advanced T cell therapies. Indeed, CAR T cells have proven to be exquisitely targetable to various antigens while there are clear examples of TCR engineered T cells driving lasting clearances of solid tumors in human patients. These technologies continue to advance, providing medical practitioners with an ever expanding toolbox of precision instruments with which to combat cancer cells.

### SUMMARY

Provided are chimeric T cell antigen receptors (TCR) comprising modified TCR chains. The modified TCR chains include fusion polypeptides having one or more heterologous antigen-binding domains fused to the extracellular domain of the TCR chain. Modified TCR chains also include chains that are modified in various other ways including e.g., chain truncation, cysteine modification, domain swapping and combinations thereof. Also provided are nucleic acids encoding the modified TCR chains as well as nucleic acids encoding the chimeric TCRs and recombinant expression vectors comprising such nucleic acids Immune cells that are genetically modified or otherwise include the described chimeric TCRs, recombinant expression vectors encoding chimeric TCRs, and/or the described nucleic acids are also provided. Methods are also provided, such as methods of killing a target cell and/or treating a subject for a condition, e.g., through the use of the described chimeric TCRs, nucleic acids, expression vectors and/or immune cells.

Aspects of the present disclosure include one or more nucleic acids encoding a chimeric T cell antigen receptor (TCR) comprising a modified α-chain and a modified β-chain that, when present in an immune cell membrane, activates the immune cell when the chimeric TCR binds an antigen, wherein: a) the modified α-chain is a fusion polypeptide comprising a heterologous antigen-binding domain, that specifically binds the antigen, fused to the extracellular domain of a TCR α-chain; or b) the modified β-chain is a fusion polypeptide comprising a heterologous antigen-binding domain, that specifically binds the antigen, fused to the extracellular domain of a TCR β-chain.

In some embodiments, one or more nucleic acids encode a chimeric TCR comprising a modified α-chain and a modified β-chain that, when present in an immune cell membrane, activates the immune cell when the chimeric TCR binds one or more antigens, wherein: a) the modified α-chain is a fusion polypeptide comprising a heterologous antigen-binding domain, that specifically binds an antigen of the one or more antigens, fused to the extracellular domain of a TCR α-chain; and b) the modified β-chain is a fusion polypeptide comprising a heterologous antigen-binding domain, that specifically binds an antigen of the one or more antigens, fused to the extracellular domain of a TCR β-chain.

In some embodiments the nucleic acid(s) include, wherein the antigen is a cancer antigen or a cell surface antigen. In some embodiments the methods include, wherein the antigen is a peptide-major histocompatibility complex (peptide-MHC). In some embodiments the nucleic acid(s) include, wherein the heterologous antigen-binding domain comprises an antibody. In some embodiments the nucleic acid(s) include, wherein the antibody is a scFv or a single domain antibody. In some embodiments the nucleic acid(s) include, wherein the heterologous antigen-binding domain comprises a ligand binding domain of a receptor. In some embodiments the nucleic acid(s) include, wherein the heterologous antigen-binding domain is fused directly to the extracellular domain. In some embodiments the nucleic acid(s) include, wherein the heterologous antigen-binding domain is fused to the extracellular domain by a linker. In some embodiments the nucleic acid(s) include, wherein the linker is less than 30 amino acids in length. In some embodiments the nucleic acid(s) include, wherein the linker is less than 20 amino acids in length. In some embodiments the nucleic acid(s) include, wherein the modified α-chain comprises a truncated α-chain, the modified β-chain comprises a truncated β-chain or the modified α-chain comprises a truncated α-chain and the modified β-chain comprises a truncated β-chain. In some embodiments the nucleic acid(s) include, wherein the modified α-chain, the modified β-chain or both the modified α-chain and the modified β-chain do not comprise a variable region. In some embodiments the nucleic acid(s) include, wherein the extracellular domain to which the heterologous antigen-binding domain is fused is a constant region of the TCR α-chain or the TCR β-chain. In some embodiments the nucleic acid(s) include, wherein the heterologous antigen-binding domain is fused directly to the constant region. In some embodiments the nucleic acid(s) include, wherein the heterologous antigen-binding domain is fused to the constant region by a linker. In some embodiments the nucleic acid(s) include, wherein the linker is less than 30 amino acids in length. In some embodiments the nucleic acid(s) include, wherein the linker is less than 20 amino acids in length. In some embodiments the nucleic acid(s) include, wherein the chimeric TCR comprises a recombinant disulfide bond between an α-chain cysteine mutation and a β-chain cysteine mutation. In some embodiments the nucleic acid(s) include, wherein the α-chain cysteine mutation is a T48C mutation and the β-chain cysteine mutation is a S57C mutation. In some embodiments the nucleic acid(s) include, wherein the modified α-chain and the modified β-chain are domain swapped modified α- and β-chains. In some embodiments the nucleic acid(s) include, wherein the domain swapped modified α- and β-chains comprise swapped α- and β-chain transmembrane regions. In some embodiments the nucleic acid(s) include, wherein the domain swapped modified α- and β-chains comprise swapped α- and β-chain cytoplasmic regions. In some embodiments the nucleic acid(s) include, wherein the domain swapped modified α- and β-chains comprise swapped α- and β-chain connecting regions. In some embodiments the nucleic acid(s) include, wherein the modified α-chain is a fusion polypeptide comprising two or more heterologous antigen-binding domains, that each specifically bind a different antigen, fused to the extracellular domain of a TCR α-chain. In some embodiments the nucleic acid(s) include, wherein the fusion polypeptide comprises a first heterologous antigen-binding domain fused to the extracellular domain of a TCR α-chain and a second heterologous antigen-binding domain fused to the first heterologous antigen-binding domain In some embodiments the nucleic acid(s) include, wherein the modified β-chain is a fusion polypeptide comprising two or more heterologous antigen-binding domains, each of which specifically binds a different antigen, fused to the extracellular domain of a TCR β-chain. In some embodiments the nucleic acid(s) include, wherein the fusion polypeptide comprises a first heterologous antigen-binding domain fused to the extracellular domain of a TCR β-chain and a second heterologous antigen-binding domain fused to the first heterologous antigen-binding domain In some embodiments the nucleic acid(s) include, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain. In some embodiments the nucleic acid(s) include, wherein the modified α-chain, the modified β-chain, or both the modified α-chain and the modified β-chain comprise a costimulatory domain In some embodiments the nucleic acid(s) include, wherein the chimeric TCR activates the immune cell to exhibit cytotoxic activity to a target cell expressing the antigen. In some embodiments the nucleic acid(s) include, wherein the activated immune cell results in a 10% or greater increase in killing of the target cell as compared to a control immune cell without the chimeric TCR. In some embodiments the nucleic acid(s) include, wherein the modified α-chain and the modified β-chain are linked into a single chain by a linking polypeptide comprising a transmembrane domain.

Aspects of the present disclosure include a recombinant expression vector comprising the nucleic acid(s) described above, wherein the expression vector comprises a promoter operably linked to a nucleotide sequence encoding the modified α-chain and a nucleotide sequence encoding the modified β-chain.

In some embodiments the recombinant expression vector includes, wherein the expression vector comprises a bicistronic-facilitating sequence between the nucleotide sequence encoding the modified α-chain and the nucleotide sequence encoding the modified β-chain. In some embodiments the recombinant expression vector includes, wherein the bicistronic-facilitating sequence comprises a furin cleavage site encoding sequence, an amino acid spacer encoding sequence and a 2A peptide encoding sequence. In some embodiments the recombinant expression vector includes, wherein the amino acid spacer encoding sequence comprises a nucleotide sequence encoding a V5 peptide. In some embodiments the recombinant expression vector includes, wherein the promoter is an inducible or conditional promoter.

Aspects of the present disclosure include a recombinant expression vector comprising the nucleic acid(s) described above, wherein the recombinant expression vector comprises a first promoter operably linked to a nucleotide sequence encoding the modified α-chain and a second promoter operably linked to a nucleotide sequence encoding the modified β-chain.

In some embodiments the recombinant expression vector includes, wherein the first promoter is an inducible or conditional promoter. In some embodiments the recombinant expression vector includes, wherein the second promoter is an inducible or conditional promoter. In some embodiments the recombinant expression vector includes, wherein the first promoter and the second promoter are copies of the same promoter.

Aspects of the present disclosure include an immune cell comprising an expression vector described above. Aspects of the present disclosure include an immune cell genetically modified to comprise a nucleic acid as described above.

Aspects of the present disclosure include a method of killing a target cell, the method comprising contacting the target cell with an immune cell as described above, wherein the target cell expresses the antigen to which the chimeric TCR binds.

In some embodiments the method includes, wherein the method is performed in vitro and the contacting comprises co-culturing the target cell and the immune cell. In some embodiments the method includes, wherein the method is performed in vivo and the contacting comprises administering the immune cell to a subject having the target cell. In some embodiments the method includes, wherein the target cell is a cancer cell and the method comprises administering to the subject an amount of the immune cells effective to treat the subject for the cancer.

Aspects of the present disclosure include a nucleic acid encoding a modified T cell antigen receptor (TCR) α-chain that, when present in a chimeric TCR within an immune cell membrane, activates the immune cell when the chimeric TCR binds an antigen, the modified TCR α-chain comprising: a heterologous antigen-binding domain; a truncated TCR α-chain extracellular domain linked to the heterologous antigen-binding domain; a TCR chain connecting region linked to the truncated TCR α-chain; a TCR chain transmembrane domain linked to the TCR chain connecting region; and a TCR chain cytoplasmic domain.

In some embodiments the nucleic acid includes, wherein the antigen is a cancer antigen. In some embodiments the nucleic acid includes, wherein the antigen is a cell surface antigen. In some embodiments the nucleic acid includes, the antigen is a peptide-major histocompatibility complex (peptide-MHC). In some embodiments the nucleic acid includes, wherein the heterologous antigen-binding domain comprises an antibody. In some embodiments the nucleic acid includes, wherein the antibody is a scFv or a single domain antibody. In some embodiments the nucleic acid includes, wherein the heterologous antigen-binding domain comprises a ligand binding domain of a receptor. In some embodiments the nucleic acid includes, wherein the heterologous antigen-binding domain is linked directly to the truncated TCR α-chain extracellular domain In some embodiments the nucleic acid includes, wherein the heterologous antigen-binding domain is linked to the truncated TCR α-chain extracellular domain by a linker. In some embodiments the nucleic acid includes, wherein the linker is less than 30 amino acids in length. In some embodiments the nucleic acid includes, wherein the linker is less than 20 amino acids in length. In some embodiments the nucleic acid includes, wherein the truncated TCR α-chain extracellular domain does not comprise a variable region. In some embodiments the nucleic acid includes, wherein the TCR chain connecting region comprises one or more cysteine substitutions. In some embodiments the nucleic acid includes, wherein the TCR chain connecting region is a TCR α-chain connecting region. In some embodiments the nucleic acid includes, wherein the one or more cysteine substitutions comprise a T48C mutation. In some embodiments the nucleic acid includes, wherein the TCR chain connecting region is a TCR β-chain connecting region. In some embodiments the nucleic acid includes, wherein the one or more cysteine substitutions comprise a S57C mutation. In some embodiments the nucleic acid includes, wherein the TCR chain transmembrane domain is a TCR α-chain transmembrane domain. In some embodiments the nucleic acid includes, wherein the TCR chain transmembrane domain is a TCR β-chain transmembrane domain In some embodiments the nucleic acid includes, wherein the TCR chain cytoplasmic domain is a TCR α-chain cytoplasmic domain. In some embodiments the nucleic acid includes, wherein the TCR chain cytoplasmic domain is a TCR β-chain cytoplasmic domain. In some embodiments the nucleic acid includes, wherein the modified TCR α-chain comprises two different heterologous antigen-binding domains. In some embodiments the nucleic acid includes, wherein the modified TCR α-chain further comprises a costimulatory domain In some embodiments the nucleic acid includes, wherein the chimeric TCR comprising the modified TCR α-chain activates the immune cell to exhibit cytotoxic activity to a target cell expressing the antigen. In some embodiments the nucleic acid includes, wherein the activated immune cell results in a 10% or greater increase in killing of the target cell as compared to a control immune cell without the chimeric TCR.

Aspects of the present disclosure include a recombinant expression vector comprising a nucleic acid as described above. Aspects of the present disclosure include an immune cell comprising the expression vector. Aspects of the present disclosure include an immune cell genetically modified to comprise the nucleic acid as described above.

Aspects of the present disclosure include an immune cell comprising: a first nucleic acid encoding a modified TCR α-chain comprising: a heterologous antigen-binding domain linked to a TCR α-chain; and a first cysteine substitution within the chain connecting region of the TCR α-chain; and a second nucleic acid encoding a modified TCR β-chain comprising a second cysteine substitution, wherein the first and second cysteine substitutions result in a recombinant disulfide bond between the modified TCR α-chain and the modified TCR β-chain. In some embodiments the immune cell includes, wherein the first cysteine substitution is a T48C mutation and the second cysteine substitution is a S57C mutation. Aspects of the present disclosure include a method of killing a target cell, the method comprising contacting the target cell with an immune cell, wherein the target cell expresses the antigen to which the chimeric TCR binds. In some embodiments the method includes, wherein the method is performed in vitro and the contacting comprises co-culturing the target cell and the immune cell. In some embodiments the method includes, wherein the method is performed in vivo and the contacting comprises administering the immune cell to a subject having the target cell. In some embodiments the method includes, wherein the target cell is a cancer cell and the method comprises administering to the subject an amount of the immune cells effective to treat the subject for the cancer.

Aspects of the present disclosure include a nucleic acid encoding a modified T cell antigen receptor (TCR) β-chain that, when present in a chimeric TCR within an immune cell membrane, activates the immune cell when the chimeric TCR binds an antigen, the modified TCR β-chain comprising: a heterologous antigen-binding domain; a truncated TCR β-chain extracellular domain linked to the heterologous antigen-binding domain; a TCR chain connecting region linked to the truncated TCR β-chain; a TCR chain transmembrane domain linked to the TCR chain connecting region; and a TCR chain cytoplasmic domain.

In some embodiments the nucleic acid includes, wherein the antigen is a cancer antigen. In some embodiments the nucleic acid includes, wherein the antigen is a cell surface antigen. In some embodiments the nucleic acid includes, the antigen is a peptide-major histocompatibility complex (peptide-MHC). In some embodiments the nucleic acid includes, wherein the heterologous antigen-binding domain comprises an antibody. In some embodiments the nucleic acid includes, wherein the antibody is a scFv or a single domain antibody. In some embodiments the nucleic acid includes, wherein the heterologous antigen-binding domain comprises a ligand binding domain of a receptor. In some embodiments the nucleic acid includes, wherein the heterologous antigen-binding domain is linked directly to the truncated TCR β-chain extracellular domain In some embodiments the nucleic acid includes, wherein the heterologous antigen-binding domain is linked to the truncated TCR β-chain extracellular domain by a linker. In some embodiments the nucleic acid includes, wherein the linker is less than 30 amino acids in length. In some embodiments the nucleic acid includes, wherein the linker is less than 20 amino acids in length. In some embodiments the nucleic acid includes, wherein the truncated TCR β-chain extracellular domain does not comprise a variable region. In some embodiments the nucleic acid includes, wherein the TCR chain connecting region comprises one or more cysteine substitutions. In some embodiments the nucleic acid includes, wherein the TCR chain connecting region is a TCR β-chain connecting region. In some embodiments the nucleic acid includes, wherein the one or more cysteine substitutions comprise a S57C mutation. In some embodiments the nucleic acid includes, wherein the TCR chain connecting region is a TCR α-chain connecting region. In some embodiments the nucleic acid includes, wherein the one or more cysteine substitutions comprise a T48C mutation. In some embodiments the nucleic acid includes, wherein the TCR chain transmembrane domain is a TCR β-chain transmembrane domain. In some embodiments the nucleic acid includes, wherein the TCR chain transmembrane domain is a TCR α-chain transmembrane domain In some embodiments the nucleic acid includes, wherein the TCR chain cytoplasmic domain is a TCR β-chain cytoplasmic domain In some embodiments the nucleic acid includes, wherein the TCR chain cytoplasmic domain is a TCR α-chain cytoplasmic domain. In some embodiments the nucleic acid includes, wherein the modified TCR β-chain comprises two different heterologous antigen-binding domains. In some embodiments the nucleic acid includes, wherein the modified TCR β-chain further comprises a costimulatory domain. In some embodiments the nucleic acid includes, wherein the chimeric TCR comprising the modified TCR β-chain activates the immune cell to exhibit cytotoxic activity to a target cell expressing the antigen. In some embodiments the nucleic acid includes, wherein the activated immune cell results in a 10% or greater increase in killing of the target cell as compared to a control immune cell without the chimeric TCR.

Aspects of the present disclosure include a recombinant expression vector comprising the nucleic acid as described above. Aspects of the present disclosure include an immune cell comprising the expression vector. Aspects of the present disclosure include an immune cell genetically modified to comprise the nucleic acid as described above.

Aspects of the present disclosure include an immune cell comprising: a first nucleic acid encoding a modified TCR β-chain comprising: a heterologous antigen-binding domain linked to a TCR β-chain; and and a first cysteine substitution within the chain connecting region of the TCR β-chain; and a second nucleic acid encoding a modified TCR α-chain comprising a second cysteine substitution, wherein the first and second cysteine substitutions result in a recombinant disulfide bond between the modified TCR β-chain and the modified TCR α-chain. In some embodiments, the immune cell includes, wherein the first cysteine substitution is a S57C mutation and the second cysteine substitution is a T48C mutation.

Aspects of the present disclosure include a method of killing a target cell, the method comprising contacting the target cell with an immune cell as described above, wherein the target cell expresses the antigen to which the chimeric TCR binds. In some embodiments the method includes, wherein the method is performed in vitro and the contacting comprises co-culturing the target cell and the immune cell. In some embodiments the method includes, wherein the method is performed in vivo and the contacting comprises administering the immune cell to a subject having the target cell. In some embodiments the method includes, wherein the target cell is a cancer cell and the method comprises administering to the subject an amount of the immune cells effective to treat the subject for the cancer.

Aspects of the present disclosure include a method of treating a subject for a condition, the method comprising: administering to the subject an effective amount of the immune cells described above in combination with an agent that ameliorates at least one side effect of the immune cells. In some embodiments the method includes, wherein the condition is cancer.

Aspects of the present disclosure include a method of treating a subject for cancer, the method comprising: administering to the subject an effective amount of the immune cells as described above in combination with a conventional cancer therapy. In some embodiments the method includes, wherein the immune cells and the conventional cancer therapy are administered in combination with an agent that ameliorates at least one side effect of the immune cells.

Aspects of the present disclosure include a chimeric T cell antigen receptor (TCR) comprising a modified α-chain and a modified β-chain that, when present in an immune cell membrane, activates the immune cell when the chimeric TCR binds an antigen, wherein: a) the modified α-chain is a fusion polypeptide comprising a heterologous antigen-binding domain, that specifically binds the antigen, fused to the extracellular domain of a TCR α-chain; or the modified β-chain is a fusion polypeptide comprising a heterologous antigen-binding domain, that specifically binds the antigen, fused to the extracellular domain of a TCR β-chain; or both the modified α-chain and the modified β-chain comprise a heterologous antigen-binding domain

In some embodiments the chimeric TCR includes, wherein the antigen is a cancer antigen. In some embodiments the chimeric TCR includes, wherein the antigen is a cell surface antigen. In some embodiments the chimeric TCR includes, wherein the antigen is a peptide-major histocompatibility complex (peptide-MHC). In some embodiments the chimeric TCR includes, wherein the heterologous antigen-binding domain comprises an antibody. In some embodiments the chimeric TCR includes, wherein the antibody is a scFv or a single domain antibody. In some embodiments the chimeric TCR includes, wherein the heterologous antigen-binding domain comprises a ligand binding domain of a receptor. In some embodiments the chimeric TCR includes, wherein the heterologous antigen-binding domain is fused directly to the extracellular domain. In some embodiments the chimeric TCR includes, wherein the heterologous antigen-binding domain is fused to the extracellular domain by a linker. In some embodiments the chimeric TCR includes, wherein the linker is less than 30 amino acids in length. In some embodiments the chimeric TCR includes, wherein the linker is less than 20 amino acids in length. In some embodiments the chimeric TCR includes, wherein the modified α-chain comprises a truncated α-chain, the modified β-chain comprises a truncated β-chain or the modified α-chain comprises a truncated α-chain and the modified β-chain comprises a truncated β-chain. In some embodiments the chimeric TCR includes, wherein the modified α-chain, the modified β-chain or both the modified α-chain and the modified β-chain do not comprise a variable region. In some embodiments the chimeric TCR includes, wherein the extracellular domain to which the heterologous antigen-binding domain is fused is a constant region of the TCR α-chain or the TCR β-chain. In some embodiments the chimeric TCR includes, wherein the heterologous antigen-binding domain is fused directly to the constant region. In some embodiments the chimeric TCR includes, wherein the heterologous antigen-binding domain is fused to the constant region by a linker. In some embodiments the chimeric TCR includes, wherein the linker is less than 30 amino acids in length. In some embodiments the chimeric TCR includes, wherein the linker is less than 20 amino acids in length. In some embodiments the chimeric TCR includes, wherein the chimeric TCR comprises a recombinant disulfide bond between an α-chain cysteine mutation and a β-chain cysteine mutation. In some embodiments the chimeric TCR includes, wherein the α-chain cysteine mutation is a T48C mutation and the β-chain cysteine mutation is a S57C mutation. In some embodiments the chimeric TCR includes, wherein the modified α-chain and the modified β-chain are domain swapped modified α- and β-chains. In some embodiments the chimeric TCR includes, wherein the domain swapped modified α- and β-chains comprise swapped α- and β-chain transmembrane regions. In some embodiments the chimeric TCR includes, wherein the domain swapped modified α- and β-chains comprise swapped α- and β-chain cytoplasmic regions. In some embodiments the chimeric TCR includes, wherein the domain swapped modified α- and β-chains comprise swapped α- and β-chain connecting regions. In some embodiments the chimeric TCR includes, wherein the modified α-chain is a fusion polypeptide comprising two or more heterologous antigen-binding domains, that each specifically bind a different antigen, fused to the extracellular domain of a TCR α-chain. In some embodiments the chimeric TCR includes, wherein the fusion polypeptide comprises a first heterologous antigen-binding domain fused to the extracellular domain of a TCR α-chain and a second heterologous antigen-binding domain fused to the first heterologous antigen-binding domain. In some embodiments the chimeric TCR includes, wherein the modified β-chain is a fusion polypeptide comprising two or more heterologous antigen-binding domains, that each specifically bind a different antigen, fused to the extracellular domain of a TCR β-chain. In some embodiments the chimeric TCR includes, wherein the fusion polypeptide comprises a first heterologous antigen-binding domain fused to the extracellular domain of a TCR β-chain and a second heterologous antigen-binding domain fused to the first heterologous antigen-binding domain In some embodiments the chimeric TCR includes, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain. In some embodiments the chimeric TCR includes, wherein the modified α-chain, the modified β-chain, or both the modified α-chain and the modified β-chain comprise a costimulatory domain In some embodiments the chimeric TCR includes, wherein the chimeric TCR activates the immune cell to exhibit cytotoxic activity to a target cell expressing the antigen. In some embodiments the chimeric TCR includes, wherein the activated immune cell results in a 10% or greater increase in killing of the target cell as compared to a control immune cell without the chimeric TCR. In some embodiments the chimeric TCR includes, wherein the modified α-chain and the modified β-chain are linked into a single chain by a linking polypeptide comprising a transmembrane domain

### BRIEF DESCRIPTION OF THE DRAWINGS

FIG. 1 depicts a schematic representation of an endogenous T cell receptor.

FIG. 2 depicts a schematic representation of an engineered T cell receptor having non-modified α+β chains.

FIG. 3 depicts a schematic representation of an engineered T cell receptor having domain-swapped α+β chains swapped at the connecting peptide-transmembrane domains.

FIG. 4 depicts a schematic representation of an engineered T cell receptor having domain-swapped α+β chains swapped at the constant-connecting peptide domains.

FIG. 5 depicts a schematic representation of construct P145 as described herein.

FIG. 6 depicts a schematic representation of construct P146 as described herein.

FIG. 7 depicts a schematic representation of construct P147 as described herein.

FIG. 8 depicts a schematic representation of construct P148 as described herein.

FIG. 9 depicts a schematic representation of construct P149 as described herein.

FIG. 10 depicts a schematic representation of construct P150 as described herein.

FIG. 11 depicts a schematic representation of construct P176 as described herein.

FIG. 12 depicts a schematic representation of construct P177 as described herein.

FIG. 13 depicts a schematic representation of construct P178 as described herein.

FIG. 14 depicts a schematic representation of construct P179 as described herein.

FIG. 15 depicts a schematic representation of construct P180 as described herein.

FIG. 16 depicts a schematic representation of construct P181 as described herein.

FIG. 17 depicts a schematic representation of construct P189 as described herein.

FIG. 18 depicts a schematic representation of construct P190 as described herein.

FIG. 19 depicts a schematic representation of construct P191 as described herein.

FIG. 20 depicts a schematic representation of construct P192 as described herein.

FIG. 21 depicts a schematic representation of construct P193 as described herein.

FIG. 22 depicts a schematic representation of construct P194 as described herein.

FIG. 23 depicts a schematic representation of construct P195 as described herein.

FIG. 24 depicts a schematic representation of construct P196 as described herein.

FIG. 25 depicts a schematic representation of construct P204 as described herein.

FIG. 26 depicts a schematic representation of construct P205 as described herein.

FIG. 27 depicts a schematic representation of construct P206 as described herein.

FIG. 28 depicts a schematic representation of construct P207 as described herein.

FIG. 29 depicts a schematic representation of construct P208 as described herein.

FIG. 30 depicts a schematic representation of construct P209 as described herein.

FIG. 31 depicts a schematic representation of construct P210 as described herein.

FIG. 32 depicts a schematic representation of construct P211 as described herein.

FIG. 33 depicts a schematic representation of construct P212 as described herein.

FIG. 34 depicts a schematic representation of construct P213 as described herein.

FIG. 35 depicts a schematic representation of construct P214 as described herein.

FIG. 36 depicts a schematic representation of construct P215 as described herein.

FIG. 37 depicts a schematic representation of construct P254 as described herein.

FIG. 38 depicts a schematic representation of construct P255 as described herein.

FIG. 39 depicts a schematic representation of construct P256 as described herein.

FIG. 40 depicts a schematic representation of construct P257 as described herein.

FIG. 41 depicts a schematic representation of construct P258 as described herein.

FIG. 42 depicts a schematic representation of construct P259 as described herein.

FIG. 43 provides Table 1 (from top to bottom, SEQ ID NOs:135-200).

FIG. 44 provides the nucleic acid sequence and certain feature locations of construct P145 (SEQ ID NO:201).

FIG. 45 provides the nucleic acid sequence and certain feature locations of construct P146 (SEQ ID NO:202).

FIG. 46 provides the nucleic acid sequence and certain feature locations of construct P147 (SEQ ID NO:203).

FIG. 47 provides the nucleic acid sequence and certain feature locations of construct P148 (SEQ ID NO:204).

FIG. 48 provides the nucleic acid sequence and certain feature locations of construct P149 (SEQ ID NO:205).

FIG. 49 provides the nucleic acid sequence and certain feature locations of construct P150 (SEQ ID NO:206).

FIG. 50 provides the nucleic acid sequence and certain feature locations of construct P176 (SEQ ID NO:207).

FIG. 51 provides the nucleic acid sequence and certain feature locations of construct P177 (SEQ ID NO:208).

FIG. 52 provides the nucleic acid sequence and certain feature locations of construct P178 (SEQ ID NO:209).

FIG. 53 provides the nucleic acid sequence and certain feature locations of construct P179 (SEQ ID NO:210).

FIG. 54 provides the nucleic acid sequence and certain feature locations of construct P180 (SEQ ID NO:211).

FIG. 55 provides the nucleic acid sequence and certain feature locations of construct P181 (SEQ ID NO:212).

FIG. 56 provides the nucleic acid sequence and certain feature locations of construct P189 (SEQ ID NO:213).

FIG. 57 provides the nucleic acid sequence and certain feature locations of construct P190 (SEQ ID NO:214).

FIG. 58 provides the nucleic acid sequence and certain feature locations of construct P191 (SEQ ID NO:215).

FIG. 59 provides the nucleic acid sequence and certain feature locations of construct P192 (SEQ ID NO:216).

FIG. 60 provides the nucleic acid sequence and certain feature locations of construct P193 (SEQ ID NO:217).

FIG. 61 provides the nucleic acid sequence and certain feature locations of construct P194 (SEQ ID NO:218).

FIG. 62 provides the nucleic acid sequence and certain feature locations of construct P195 (SEQ ID NO:219).

FIG. 63 provides the nucleic acid sequence and certain feature locations of construct P196 (SEQ ID NO:220).

FIG. 64 provides the nucleic acid sequence and certain feature locations of construct P204 (SEQ ID NO:221).

FIG. 65 provides the nucleic acid sequence and certain feature locations of construct P205 (SEQ ID NO:222).

FIG. 66 provides the nucleic acid sequence and certain feature locations of construct P206 (SEQ ID NO:223).

FIG. 67 provides the nucleic acid sequence and certain feature locations of construct P207 (SEQ ID NO:224).

FIG. 68 provides the nucleic acid sequence and certain feature locations of construct P208 (SEQ ID NO:225).

FIG. 69 provides the nucleic acid sequence and certain feature locations of construct P209 (SEQ ID NO:226).

FIG. 70 provides the nucleic acid sequence and certain feature locations of construct P210 (SEQ ID NO:227).

FIG. 71 provides the nucleic acid sequence and certain feature locations of construct P211 (SEQ ID NO:228).

FIG. 72 provides the nucleic acid sequence and certain feature locations of construct P212 (SEQ ID NO:229).

FIG. 73 provides the nucleic acid sequence and certain feature locations of construct P213 (SEQ ID NO:230).

FIG. 74 provides the nucleic acid sequence and certain feature locations of construct P214 (SEQ ID NO:231).

FIG. 75 provides the nucleic acid sequence and certain feature locations of construct P215 (SEQ ID NO:232).

FIG. 76 provides the nucleic acid sequence and certain feature locations of construct P254 (SEQ ID NO:233).

FIG. 77 provides the nucleic acid sequence and certain feature locations of construct P255 (SEQ ID NO:234).

FIG. 78 provides the nucleic acid sequence and certain feature locations of construct P256 (SEQ ID NO:235).

FIG. 79 provides the nucleic acid sequence and certain feature locations of construct P257 (SEQ ID NO:236).

FIG. 80 provides the nucleic acid sequence and certain feature locations of construct P258 (SEQ ID NO:237).

FIG. 81 provides the nucleic acid sequence and certain feature locations of construct P259 (SEQ ID NO:238).

FIG. 82 depicts immune cell activation and antigen-specific target cell killing by human CD8 T cells transduced to express a chimeric TCR according to an embodiment of the disclosure.

FIG. 83 depicts immune cell activation and antigen-specific target cell killing by human CD8 T cells transduced to express various chimeric TCRs according embodiments of the disclosure.

FIG. 84 depicts immune cell activation by Jurkat T cells transduced to express a chimeric TCR according to an embodiment of the disclosure.

FIG. 85 provides quantification of the transduction of T cells with various chimeric TCRs, as compared to untransduced and chimeric antigen receptor (CAR) controls, as described herein.

FIG. 86 depicts the cell surface expression various chimeric TCRs, as compared to untransduced and chimeric antigen receptor (CAR) controls, as described herein.

FIG. 87 depicts a comparison of the cell surface expression of chimeric TCRs (synTCRs) having paired and unpaired modified alpha and beta TCR chains.

FIG. 88 provides quantification of the cell surface expression of various chimeric TCRs (synTCRs), as compared to untransduced and chimeric antigen receptor (CAR) controls, as described herein.

FIG. 89 provides the FACS profiles utilized in the quantification presented in FIG. 88.

FIG. 90 provides a comparison of the in vivo efficacy of CAR T cells versus synTCR T cells.

FIG. 91 shows comparable survival of tumor carrying mice treated with CAR T cells as compared to tumor carrying mice treated with synTCR T cells.

FIG. 92 demonstrates CD19-specific immune activation by synTCR T cells expressing either an anti-CD19 scFv alpha chain synTCR or an anti-CD19 scFv beta chain synTCR.

FIG. 93 demonstrates CD22-specific immune activation by synTCR T cells expressing either an anti-CD22 scFv alpha chain synTCR or an anti-CD22 scFv beta chain synTCR.

FIG. 94 demonstrates CD22-specific immune activation by T cells expressing a synTCR with an anti-CD22 scFv on both alpha and beta chains as well as CD19-specific immune activation by T cells expressing a synTCR with an anti-CD19 scFv on both alpha and beta chains.

FIG. 95 shows the expression by primary human CD8 T cells of an anti-GFP synTCR with a 41BB costimulatory domain fused intracellularly to the truncated TCR alpha chain.

FIG. 96 demonstrates antigen-specific immune activation by T cells transduced with the costimulatory domain containing synTCR depicted and expressed in FIG. 95.

FIG. 97 provides the nucleic acid sequence and certain feature locations of construct p286 (SEQ ID NO:239).

FIG. 98 provides the nucleic acid sequence and certain feature locations of construct p345 (SEQ ID NO:240).

FIG. 99 provides the nucleic acid sequence and certain feature locations of construct p353 (SEQ ID NO:241).

FIG. 100 provides the nucleic acid sequence and certain feature locations of construct p354 (SEQ ID NO:242).

FIG. 101 provides the nucleic acid sequence and certain feature locations of construct p435 (SEQ ID NO:243).

FIG. 102 provides the nucleic acid sequence and certain feature locations of construct p436 (SEQ ID NO:244).

FIG. 103 provides the nucleic acid sequence and certain feature locations of construct p312 (SEQ ID NO:245).

### DEFINITIONS

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

“Operably linked” refers to a juxtaposition wherein the components so described are in a relationship permitting them to function in their intended manner For instance, a promoter is operably linked to one or more coding sequences if the promoter affects the transcription or expression of the one or more coding sequences to which it is linked.

A “biological sample” encompasses a variety of sample types obtained from an individual or a population of individuals and can be used in various ways, including e.g., the isolation of cells or biological molecules, diagnostic assays, etc. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples. Accordingly, biological samples may be cellular samples or acellular samples.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide or nucleic acid is one that has been identified and separated and/or recovered from a component of its natural environment. Contaminant components of its natural environment are materials that would interfere with diagnostic or therapeutic uses for the polypeptide or nucleic acid, and may include enzymes, hormones, and other proteinaceous or nonproteinaceous solutes. In some embodiments, a polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, nanobodies, single-domain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁷ M, e.g., binding with an affinity of 10⁻⁶M, 10⁻⁵ M, 10 ⁻⁴ M, etc.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides chimeric T cell antigen receptors (TCRs) that include modified TCR chains. As described in more detail below, “modified TCR chains” encompass any TCR chain, e.g., TCR alpha or TCR beta, that has been modified from its naturally occurring form.

In some instances, TCRs containing such modified chains may be referred to as engineered TCRs, recombinant TCRs or synthetic TCRs (including “synTCR”).

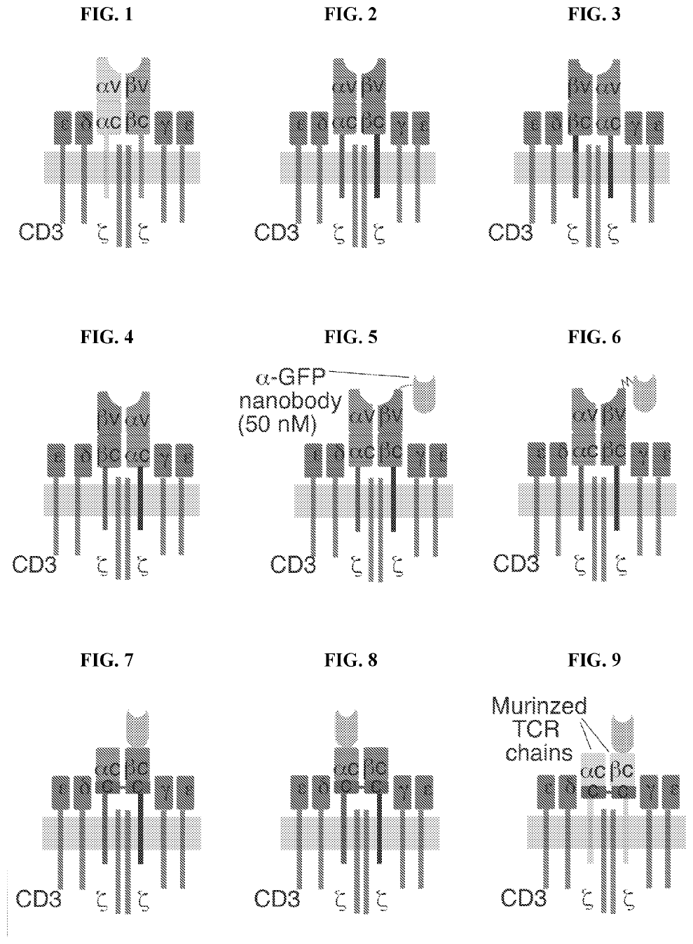

A schematic representation of an endogenous TCR complex is provided in FIG. 1. The TCR complex is a disulfide-linked membrane-anchored heterodimeric protein normally consisting of the highly variable alpha (α) and beta (β) chains expressed as part of a complex with CD3 chain molecules. Many native TCRs exist in heterodimeric αβ or γ67 forms. The complete endogenous TCR complex in heterodimeric αβ form, as shown in FIG. 1, includes eight chains, namely an alpha chain (referred to herein as TCRα or TCR alpha), beta chain (referred to herein as TCRβ or TCR beta), delta chain, gamma chain, two epsilon chains and two zeta chains. The TCR will be generally referred to herein by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane will include reference to the fully or partially assembled TCR complex.

Recombinant or engineered individual TCR chains and TCR complexes have been developed. Individual recombinant TCR chains may be generally referred to herein as modified TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

In some embodiments, chimeric TCRs of the present disclosure include paired modified TCR chains, including paired modified TCR alpha and modified TCR beta chains where the subject chimeric TCR includes both a modified TCRα chain and modified TCRβ chain. One example of paired modified alpha and beta chains would include where the modified chains are full length and associate with endogenous delta, gamma, epsilon and zeta chains (e.g., as depicted in FIG. 2). Full length examples of modified chains also include domain swapped chains, e.g., where domains are swapped between alpha and beta chains at the transmembrane domain (see e.g., FIG. 3) or at the constant domain (see e.g., FIG. 4).

In some instances, paired chains result in preferential pairing between the modified chains while also limiting pairing of the modified chains with an endogenously expressed TCR alpha or beta chain. For example, in some instances, paired domain swapped chains will preferentially pair with each other while limiting pairing of either of the domain swapped chains with an endogenous TCR chain. In some instances, paired truncated chains will preferentially pair with each other while limiting pairing of either of the truncated chains with an endogenous TCR chain. In some instances, cysteine modified chains will preferentially pair with each other while limiting pairing of either of the cysteine modified chains with an endogenous TCR chain.

In some instances, a chimeric TCR of the present disclosure may include a modified TCR alpha chain. Any convenient domain(s) of a TCR alpha chain may find use in constructing a modified TCR alpha chain for use in a chimeric TCR of the present disclosure. In some instances, the TCR alpha chain or one or more domains thereof will be a mammalian TCR alpha chain or a mammalian TCR alpha chain domain In some instances, the mammalian TCR alpha chain or one or more domains thereof will be a rodent TCR alpha chain or a rodent TCR alpha chain domain. In some instances, the rodent TCR alpha chain or one or more domains thereof will be a mouse TCR alpha chain or a mouse TCR alpha chain domain In some instances, the mammalian TCR alpha chain or one or more domains thereof will be a primate TCR alpha chain (e.g., a non-human primate TCR alpha chain) or a primate TCR alpha chain domain (e.g., a non-human primate TCR alpha chain domain) In some instances, the primate TCR alpha chain or one or more domains thereof will be a human TCR alpha chain or a human TCR alpha chain domain

Useful TCR alpha chain domains include but are not limited to e.g., an alpha variable domain, an alpha constant domain, an alpha transmembrane domain, an alpha connecting peptide domain, and the like. In some instances, useful TCR alpha chain domains include but are not limited to e.g., a human alpha variable domain, a human alpha constant domain, a human alpha transmembrane domain, a human alpha connecting peptide domain, and the like.

As used herein the term “variable domain” is understood to encompass all amino acids of a given TCR which are not included within the constant domain as encoded by the TRAC gene for TCR a chains and either the TRBC1 or TRBC2 for TCR β chains as described in, e.g., T cell receptor Factsbook, (2001) LeFranc and LeFranc, Academic Press.

In some instances, a chimeric TCR of the present disclosure may include an alpha variable domain, where such domain may have 75% or more sequence identity, including e.g., 80% or more, 85% or more, 90% or more, 95% or more, 96% or more, 97% or more, 98% or more, 99% or more or 100% sequence identity to the following human alpha chain variable region sequence:

(SEQ ID NO: 1)
METLLGLLILWLQLQWVSSKQEVTQIPAALSVPEGENLVLNCSFTDSAIY
NLQWFRQDPGKGLTSLLLIQSSQREQTSGRLNASLDKSSGRSTLYIAASQ
PGDSATYLCAVRPTSGGSYIPTFGRGTSLIVHP.

In some instances, for example in the case of a truncated TCR alpha chain, a chimeric TCR of the present disclosure may not have (i.e., may exclude) an alpha chain variable domain, including e.g., wherein an alpha chain of the subject chimeric TCR excludes all or most of (including e.g., 75% or more of, 80% or more of, 85% or more of, 90% or more of, 95% or more of, 96% or more of, 97% or more of, 98% or more of, 99% or more of or 100% of) a TCR alpha chain variable domain, including e.g., the domain for which an amino acid sequence is provided above.

In some instances, a chimeric TCR of the present disclosure may include a modified TCR beta chain. Any convenient domain(s) of a TCR beta chain may find use in constructing a modified TCR beta chain for use in a chimeric TCR of the present disclosure. In some instances, the TCR beta chain or one or more domains thereof will be a mammalian TCR beta chain or a mammalian TCR beta chain domain In some instances, the mammalian TCR beta chain or one or more domains thereof will be a rodent TCR beta chain or a rodent TCR beta chain domain In some instances, the rodent TCR beta chain or one or more domains thereof will be a mouse TCR beta chain or a mouse TCR beta chain domain In some instances, the mammalian TCR beta chain or one or more domains thereof will be a primate TCR beta chain or a primate TCR beta chain domain In some instances, the primate TCR beta chain or one or more domains thereof will be a human TCR beta chain or a human TCR beta chain domain.

Useful TCR beta chain domains include but are not limited to e.g., a beta variable domain, a beta constant domain, a beta transmembrane domain, a beta connecting peptide domain, and the like. In some instances, useful TCR beta chain domains include but are not limited to e.g., a human beta variable domain, a human beta constant domain, a human beta transmembrane domain, a human beta connecting peptide domain, and the like.

In some instances, a chimeric TCR of the present disclosure may include a beta variable domain, where such domain may have 75% or more sequence identity, including e.g., 80% or more, 85% or more, 90% or more, 95% or more, 96% or more, 97% or more, 98% or more, 99% or more or 100% sequence identity to the following human beta chain variable region sequence:

(SEQ ID NO: 2)
MSIGLLCCAALSLLWAGPVNAGVTQTPKFQVLKTGQSMTLQCAQDMNHEY
MSWYRQDPGMGLRLIHYSVGAGITDQGEVPNGYNVSRSTTEDFPLRLLSA
APSQTSVYFCASSYVGNTGELFFGEGSRLTVL.

In some instances, for example in the case of a truncated TCR beta chain, a chimeric TCR of the present disclosure may not have (i.e., may exclude) a beta chain variable domain, including e.g., wherein a beta chain of the subject chimeric TCR excludes all or most of (including e.g., 75% or more of, 80% or more of, 85% or more of, 90% or more of, 95% or more of, 96% or more of, 97% or more of, 98% or more of, 99% or more of or 100% of) a TCR beta chain variable domain, including e.g., the domain for which an amino acid sequence is provided above.

In some instances, a chimeric TCR of the present disclosure may include an alpha constant domain, where such domain may have 75% or more sequence identity, including e.g., 80% or more, 85% or more, 90% or more, 95% or more, 96% or more, 97% or more, 98% or more, 99% or more or 100% sequence identity to the following human alpha chain constant region sequence:

(SEQ ID NO: 3)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGFNLLMTLRLWSS.

In some instances, a chimeric TCR of the present disclosure may include an alpha constant domain, where such domain may have 75% or more sequence identity, including e.g., 80% or more, 85% or more, 90% or more, 95% or more, 96% or more, 97% or more, 98% or more, 99% or more or 100% sequence identity to the following mouse alpha chain constant region sequence:

(SEQ ID NO: 4)
PYIQNPEPAVYQLKDPRSQDSTLCLFTDFDSQINVPKTMESGTFITDKTV
LDMKAMDSKSNGAIAWSNQTSFTCQDIFKETNATYPSSDVPCDATLTEKS
FETDMNLNFQNLSVMGLRILLLKVAGFNLLMTLRLWSS.

In some instances, a chimeric TCR of the present disclosure may not have (i.e., may exclude) some portion of an alpha chain constant region, including but not limited to e.g., where the alpha chain constant region is truncated at either end by one or more amino acids, including from 1 to 5 aa or more including e.g., by 1 aa, by 2 aa, by 3 aa, by 4 aa, by 5 aa, etc.

In some instances, a chimeric TCR of the present disclosure may include a beta constant domain, where such domain may have 75% or more sequence identity, including e.g., 80% or more, 85% or more, 90% or more, 95% or more, 96% or more, 97% or more, 98% or more, 99% or more or 100% sequence identity to the following human beta chain constant region sequence:

(SEQ ID NO: 5)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF.

In some instances, a chimeric TCR of the present disclosure may include a beta constant domain, where such domain may have 75% or more sequence identity, including e.g., 80% or more, 85% or more, 90% or more, 95% or more, 96% or more, 97% or more, 98% or more, 99% or more or 100% sequence identity to the following mouse beta chain constant region sequence:

(SEQ ID NO: 6)
EDLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS.

In some instances, a chimeric TCR of the present disclosure may not have (i.e., may exclude) some portion of a beta chain constant region, including but not limited to e.g., where the beta chain constant region is truncated at either end by one or more amino acids, including from 1 to 5 aa or more including e.g., by 1 aa, by 2 aa, by 3 aa, by 4 aa, by 5 aa, etc.

The overall length of a subject TCR chain may vary and may range from less than 20 amino acids to 1000 amino acid or more, including but not limited to e.g., from 20 aa to 1000 aa, from 30 aa to 1000 aa, from 40 aa to 1000 aa, from 50 aa to 1000 aa, from 60 aa to 1000 aa, from 70 aa to 1000 aa, from 80 aa to 1000 aa, from 90 aa to 1000 aa, from 100 aa to 1000 aa, from 150 aa to 1000 aa, from 200 aa to 1000 aa, from 250 aa to 1000 aa, from 300 aa to 1000 aa, from 350 aa to 1000 aa, from 400 aa to 1000 aa, from 450 aa to 1000 aa, from 500 aa to 1000 aa, from 550 aa to 1000 aa, from 600 aa to 1000 aa, from 650 aa to 1000 aa, from 700 aa to 1000 aa, from 750 aa to 1000 aa, from 800 aa to 1000 aa, from 850 aa to 1000 aa, from 900 aa to 1000 aa, from 950 aa to 1000 aa, from 20 aa to 950 aa, from 20 aa to 900 aa, from 20 aa to 850 aa, from 20 aa to 800 aa, from 20 aa to 750 aa, from 20 aa to 700 aa, from 20 aa to 650 aa, from 20 aa to 600 aa, from 20 aa to 550 aa, from 20 aa to 500 aa, from 20 aa to 450 aa, from 20 aa to 400 aa, from 20 aa to 350 aa, from 20 aa to 300 aa, from 20 aa to 250 aa, from 20 aa to 200 aa, from 20 aa to 150 aa, from 20 aa to 100 aa, from 30 aa to 950 aa, from 40 aa to 900 aa, from 50 aa to 850 aa, from 60 aa to 800 aa, from 70 aa to 750 aa, from 80 aa to 700 aa, from 90 aa to 650 aa, from 100 aa to 600 aa, from 150 aa to 550 aa, from 200 aa to 500 aa, from 250 aa to 450 aa, from 300 aa to 400 aa, from 20 aa to 500 aa, from 30 aa to 500 aa, from 40 aa to 500 aa, from 50 aa to 500 aa, from 60 aa to 500 aa, from 70 aa to 500 aa, from 80 aa to 500 aa, from 90 aa to 500 aa, from 100 aa to 500 aa, from 150 aa to 500 aa, from 200 aa to 500 aa, from 250 aa to 500 aa, from 300 aa to 500 aa, from 350 aa to 500 aa, from 400 aa to 500 aa, from 450 aa to 500 aa, from 150 aa to 950 aa, from 150 aa to 900 aa, from 150 aa to 850 aa, from 150 aa to 800 aa, from 150 aa to 750 aa, from 150 aa to 700 aa, from 150 aa to 650 aa, from 150 aa to 600 aa, from 150 aa to 550 aa, from 150 aa to 500 aa, from 150 aa to 450 aa, from 150 aa to 400 aa, from 150 aa to 350 aa, from 150 aa to 300 aa, from 150 aa to 250 aa, from 150 aa to 200 aa, from 500 aa to 950 aa, from 500 aa to 900 aa, from 500 aa to 850 aa, from 500 aa to 800 aa, from 500 aa to 750 aa, from 500 aa to 700 aa, from 500 aa to 650 aa, from 500 aa to 600 aa, etc., where the overall length of the subject TCR chain may include or exclude a linked antigen binding domain where present.

As described in more detail below, the subject alpha and/or beta chains included in a chimeric TCR may be modified from their naturally occurring form in one or more ways including but not limited to e.g., chain truncation, cysteine modification, domain swapping, addition of a heterologous signaling domain (e.g., a heterologous co-stimulatory domain), etc. Naturally occurring alpha and beta chains that may be modified for use in the subject chimeric TCRs are not limited to those specifically disclosed above and include any naturally occurring mammalian alpha or beta TCR chain with the appropriate functionality.

### Chimeric T Cell Antigen Receptors (TCRs)

As summarized above, chimeric T cell receptors of the present disclosure will generally include TCRs having modified alpha and beta chains wherein at least one of the chains is fused to a heterologous antigen binding domain In some instances, a modified alpha chain of a chimeric TCR of the present disclosure may, with the exception of a heterologous antigen binding domain fused to the alpha chain, be otherwise unmodified from its naturally occurring form. In some instances, a modified alpha chain of a chimeric TCR of the present disclosure may not include a fused heterologous antigen binding domain but may be modified in some other way, including e.g., chain truncation, cysteine modification, domain swapping, addition of a heterologous co-stimulatory domain, etc. In some instances, a modified alpha chain of a chimeric TCR of the present disclosure may include both: a fused heterologous antigen binding domain and a further modification, including but not limited to e.g., chain truncation, cysteine modification, domain swapping, addition of a heterologous co-stimulatory domain, etc., including combinations thereof.

In some instances, a modified beta chain of a chimeric TCR of the present disclosure may, with the exception of a heterologous antigen binding domain fused to the beta chain, be otherwise unmodified from its naturally occurring form. In some instances, a modified beta chain of a chimeric TCR of the present disclosure may not include a fused heterologous antigen binding domain but may be modified in some other way, including e.g., chain truncation, cysteine modification, domain swapping, addition of a heterologous co-stimulatory domain, etc. In some instances, a modified beta chain of a chimeric TCR of the present disclosure may include both: a fused heterologous antigen binding domain and a further modification, including but not limited to e.g., chain truncation, cysteine modification, domain swapping, addition of a heterologous co-stimulatory domain, etc., including combinations thereof.

A chimeric TCR of the present disclosure may, in some instances, also include one or more epsilon, delta, gamma and/or zeta chains, modified or unmodified. For example, where a subject chimeric TCR is expressed from a nucleic acid, the nucleic acid may include one or more sequences encoding for one or more of an epsilon chain, a delta chain, a gamma chain and/or a zeta chain. In some instances, a chimeric TCR may not include one or more epsilon, delta, gamma and/or zeta chains and may instead rely upon endogenously expressed epsilon, delta, gamma and/or zeta chains. For example, where a subject chimeric TCR is expressed from a nucleic acid, the nucleic acid may not include one or more sequences encoding for one or more of an epsilon chain, a delta chain, a gamma chain and/or a zeta chain.

In some instances, a chimeric TCR of the present disclosure may include a TCR chain having or excluding one or more domains of a particular TCR chain (e.g., alpha or beta) relative to the naturally occurring counterpart. Such chains may be recombinantly produced or partly or completely synthetic. For example, in some instances a subject chain of chimeric TCR may include or exclude a variable region (e.g., an alpha chain variable region or a beta chain variable region). In some instances, a subject chain of chimeric TCR may include or exclude one, two or three of the naturally present complementarity determining regions (CDRs). In some instances, a subject chain of chimeric TCR may include or exclude all or a portion of an alpha or beta chain framework region. In some instances, a subject chain of chimeric TCR may include or exclude a beta chain HV4 hypervariability region.

In some instances, a subject chain of chimeric TCR may include or exclude a portion of the constant region (e.g., an alpha chain constant region or a beta chain constant region). For example, a subject chain of a chimeric TCR may include or exclude one or more of an alpha chain connecting peptide, a beta chain connecting peptide, an alpha chain transmembrane domain or a beta chain transmembrane domain.

In some instances, a subject chain of chimeric TCR may include or exclude an alpha connecting peptide of the TCR alpha constant region. In some instances, the chain includes an amino acid sequence having 75% or more sequence identity, including e.g., 80% or more, 85% or more, 90% or more, 95% or more, 96% or more, 97% or more, 98% or more, 99% or more or 100% sequence identity to the following human alpha connecting peptide sequence: CDVKLVEKSFETDTNLNFQN (SEQ ID NO:7). In some instances, the subject chimeric TCR chain excludes the human alpha connecting peptide sequence or a sequence having 85% or greater, e.g., 90% or greater, 95% or greater, 96% or greater, 97% or greater, 98% or greater, 99% or greater, sequence identity to the above human alpha connecting peptide sequence.

In some instances, a subject chain of chimeric TCR may include or exclude a transmembrane domain of the TCR alpha constant region. In some instances, the chain includes an amino acid sequence having 75% or more sequence identity, including e.g., 80% or more, 85% or more, 90% or more, 95% or more, 96% or more, 97% or more, 98% or more, 99% or more or 100% sequence identity to the following human alpha a transmembrane domain sequence: VIGFRILLLKVAGFNLLMTL (SEQ ID NO:8). In some instances, the subject chimeric TCR chain excludes the human alpha a transmembrane domain sequence or a sequence having 85% or greater, e.g., 90% or greater, 95% or greater, 96% or greater, 97% or greater, 98% or greater, 99% or greater, sequence identity to the above human alpha a transmembrane domain

In some instances, a subject chain of chimeric TCR may include or exclude a beta connecting peptide of the TCR beta constant region. In some instances, the chain includes an amino acid sequence having 75% or more sequence identity, including e.g., 80% or more, 85% or more, 90% or more, 95% or more, 96% or more, 97% or more, 98% or more, 99% or more or 100% sequence identity to the following human beta connecting peptide sequence: CGFTSVSYQQGVLSAT (SEQ ID NO:9). In some instances, the subject chimeric TCR chain excludes the human beta connecting peptide sequence or a sequence having 85% or greater, e.g., 90% or greater, 95% or greater, 96% or greater, 97% or greater, 98% or greater, 99% or greater, sequence identity to the above human beta connecting peptide sequence.

In some instances, a subject chain of chimeric TCR may include or exclude a transmembrane domain of the TCR beta constant region. In some instances, the chain includes an amino acid sequence having 75% or more sequence identity, including e.g., 80% or more, 85% or more, 90% or more, 95% or more, 96% or more, 97% or more, 98% or more, 99% or more or 100% sequence identity to the following human beta a transmembrane domain sequence: ILLGKATLYAVLVSALVLMAM (SEQ ID NO:10). In some instances, the subject chimeric TCR chain excludes the human beta a transmembrane domain sequence or a sequence having 85% or greater, e.g., 90% or greater, 95% or greater, 96% or greater, 97% or greater, 98% or greater, 99% or greater, sequence identity to the above human beta a transmembrane domain

In some instances, a subject chain of chimeric TCR may include or exclude all or a portion of a cytoplasmic domain of a TCR alpha chain or a TCR beta chain. In some instances, a subject chain of chimeric TCR may include or exclude all or a portion of an extracellular domain (e.g., including both the extracellular variable and extracellular constant regions) of a TCR alpha chain or a TCR beta chain. In some instances, TCR chains, including TCR alpha chain and TCR beta chains, may be referred to herein as “truncated” or as having a “truncation”. A “truncated chain”, as used herein, generally refers to any chain that is not full length (i.e., is of a length shorter than that of the corresponding wild-type or naturally occurring chain). Truncated chains may be N-terminal truncations (including where the amino acids have been removed from the N-terminal end but the C-terminal end has not been truncated), C-terminal truncations (including where the amino acids have been removed from the C-terminal end but the N-terminal end has not been truncated) or may include a combination of N-terminal and C-terminal truncation (including where amino acids have been removed from both the N-terminal and C-terminal ends).

Any TCR chain may be truncated and TCR chains may be truncated at any convenient and appropriate location along the length of the subject chain. For example, in some instances, a subject truncated TCR chain is a truncated TCR alpha chain, including where the truncated TCR alpha chain is truncated within the variable region, at the boundary between the variable region and the constant region, within the constant region, at the boundary between the constant region and the transmembrane domain, within the transmembrane domain, etc. In some embodiments, a chimeric TCR of the present disclosure includes a truncated TCR alpha chain that has been truncated to remove (i.e., exclude) the TCR alpha variable domain or a portion thereof, the constant domain or a portion thereof, the connecting peptide or a portion thereof or a portion of the transmembrane domain. In some instances, a truncated TCR alpha chain may be truncated to include only the transmembrane domain and the intracellular domain, i.e., to exclude the variable domain, the constant domain and the connecting peptide region.

In some instances, a subject truncated TCR chain is a truncated TCR beta chain, including where the truncated TCR beta chain is truncated within the variable region, at the boundary between the variable region and the constant region, within the constant region, at the boundary between the constant region and the transmembrane domain, within the transmembrane domain, etc. In some embodiments, a chimeric TCR of the present disclosure includes a truncated TCR beta chain that has been truncated to remove (i.e., exclude) the TCR beta variable domain or a portion thereof, the constant domain or a portion thereof, the connecting peptide or a portion thereof or a portion of the transmembrane domain In some instances, a truncated TCR beta chain may be truncated to include only the transmembrane domain and the intracellular domain, i.e., to exclude the variable domain, the constant domain and the connecting peptide region.

In some instances, a subject chimeric TCR may include a pair of truncated TCR chains. For example, in some instances, a chimeric TCR of the present disclosure may include an alpha and beta chain pair that includes a truncated TCR alpha chain and a truncated TCR beta chain. Pairs of truncated chains may or may not be truncated at corresponding positions along the chain. For example, in some instances, a pair of truncated chains may be truncated at non-corresponding positions. In some instances, a pair of truncated chains may be truncated at corresponding positions, including e.g., where the individual chains of an alpha and beta pair are both truncated at the junction between the variable region and the constant region, and the like.

The overall length of a subject truncated TCR chain may vary and may range from less than 20 amino acids to 500 amino acid or more, including but not limited to e.g., from 20 aa to 500 aa, from 30 aa to 500 aa, from 40 aa to 500 aa, from 50 aa to 500 aa, from 60 aa to 500 aa, from 70 aa to 500 aa, from 80 aa to 500 aa, from 90 aa to 500 aa, from 100 aa to 500 aa, from 150 aa to 500 aa, from 200 aa to 500 aa, from 250 aa to 500 aa, from 300 aa to 500 aa, from 350 aa to 500 aa, from 400 aa to 500 aa, from 450 aa to 500 aa, from 20 aa to 450 aa, from 20 aa to 400 aa, from 20 aa to 350 aa, from 20 aa to 300 aa, from 20 aa to 250 aa, from 20 aa to 200 aa, from 20 aa to 150 aa, from 20 aa to 100 aa, from 30 aa to 450 aa, from 40 aa to 400 aa, from 50 aa to 350 aa, from 60 aa to 300 aa, from 70 aa to 250 aa, from 80 aa to 200 aa, from 90 aa to 150 aa, from 100 aa to 500 aa, from 150 aa to 450 aa, from 200 aa to 400 aa, from 250 aa to 350 aa, from 20 aa to 250 aa, from 30 aa to 250 aa, from 40 aa to 250 aa, from 50 aa to 250 aa, from 60 aa to 250 aa, from 70 aa to 250 aa, from 80 aa to 250 aa, from 90 aa to 250 aa, from 100 aa to 250 aa, from 150 aa to 250 aa, from 200 aa to 250 aa, from 150 aa to 500 aa, from 150 aa to 450 aa, from 150 aa to 400 aa, from 150 aa to 350 aa, from 150 aa to 300 aa, from 150 aa to 250 aa, from 150 aa to 200 aa, from 250 aa to 450 aa, from 250 aa to 400 aa, from 250 aa to 350 aa, from 250 aa to 300 aa, etc., where the overall length of the subject truncated TCR chain may include or exclude a linked antigen binding domain where present.

As noted above, one or more chains of a chimeric TCR of the present disclosure may be a fusion protein, including e.g., where the one or more chains is fused with a heterologous domain. Various heterologous domains may be fused to the subject TCR chains, including e.g., heterologous antigen binding domains, heterologous signaling-related domains (e.g., co-stimulatory domains), and the like. Such domains may be fused to the subject chain by any convenient means and may, in some instances, be a terminal fusion (i.e., fused to the N- or C-terminus of the polypeptide). In some instances, the heterologous domain may be fused to the end of truncated chain, including e.g., the new N- or C-terminus resulting from a truncation.

In some instances, a chimeric TCR may include a single fused heterologous domain In some instances a chimeric TCR may include multiple fused heterologous domains, including but not limited to e.g., 2 or more fused domains, 3 or more fused domains, 4 or more fused domains, 5 or more fused domains, 6 or more fused domains, 7 or more fused domains, 8 or more fused domains, 9 or more fused domains, 10 or more fused domains, etc.

In some instances, a chimeric TCR may include a single fused heterologous antigen binding domain In some instances a chimeric TCR may include multiple fused heterologous antigen binding domains, including but not limited to e.g., 2 or more fused antigen binding domains, 3 or more fused antigen binding domains, 4 or more fused antigen binding domains, 5 or more fused antigen binding domains, 6 or more fused antigen binding domains, 7 or more fused antigen binding domains, 8 or more fused antigen binding domains, 9 or more fused antigen binding domains, 10 or more fused antigen binding domains, etc.

As such, where a chimeric TCR includes two or more fused heterologous domains, the plurality of domains may be fused to a single chain of the chimeric TCR. In some instances, where a chimeric TCR includes two or more fused heterologous domains, both chains of the chimeric TCR may include at least one fused heterologous domain, including where the number of domains fused to each chain are the same or different. In some instances, a chimeric TCR may include a first heterologous domain fused to a modified alpha chain and a second heterologous domain fused to a modified beta chain where the first and second heterologous domains are the same or different. In some instances, the first and second heterologous domains may be antigen-binding domains where the first and second antigen-binding domains may be the same or different and may be directed to the same antigen or to different antigens.

In some instances, a modified alpha or beta chain may include a single fused heterologous domain. In some instances, a modified alpha or beta chain may include multiple fused heterologous domains, including but not limited to e.g., 2 or more fused domains, 3 or more fused domains, 4 or more fused domains, 5 or more fused domains, 6 or more fused domains, 7 or more fused domains, 8 or more fused domains, 9 or more fused domains, 10 or more fused domains, etc.

In some instances, a modified alpha or beta chain may include a single fused heterologous antigen binding domain. In some instances, a modified alpha or beta chain may include multiple fused heterologous antigen binding domains, including but not limited to e.g., 2 or more fused antigen binding domains, 3 or more fused antigen binding domains, 4 or more fused antigen binding domains, 5 or more fused antigen binding domains, 6 or more fused antigen binding domains, 7 or more fused antigen binding domains, 8 or more fused antigen binding domains, 9 or more fused antigen binding domains, 10 or more fused antigen binding domains, etc.

Fusion of heterologous domains to a chain of a chimeric TCR may be achieved with or without the use of a linker (i.e., linking polypeptide). Suitable linkers, including non-limiting examples, are described in more detail below. In some instances, a linker used in joining two polypeptides or domains may be less than 50 amino acids in length, including e.g., where the subject linker is 45 aa or less, 40 aa or less, 35 aa or less, 34 aa or less, 33 aa or less, 32 aa or less, 31 aa or less, 30 aa or less, 29 aa or less, 28 aa or less, 27 aa or less, 26 aa or less, 25 aa or less, 24 aa or less, 23 aa or less, 22 aa or less, 21 aa or less, 20 aa or less, 19 aa or less, 18 aa or less, 17 aa or less, 16 aa or less, 15 aa or less, 14 aa or less, 13 aa or less, 12 aa or less, 11 aa or less, 10 aa or less, 9 aa or less, 8 aa or less, 7 aa or less, 6 aa or less, 5 aa or less, 4 aa or less, 3 aa or less, 2 aa or less or 1 aa. In some embodiments, a heterologous antigen binding domain may be fused to the constant domain of a TCR alpha chain by way of a peptide linker. In some embodiments, a heterologous antigen binding domain may be fused to the constant domain of a TCR beta chain by way of a peptide linker.

In some instances, a subject heterologous domain may be fused directly to a terminus or domain of a TCR chain without the use of a linker (i.e., where no intervening amino acids are present between the two joined polypeptides or domains). In some embodiments, a heterologous antigen binding domain may be directly fused to the constant domain of a TCR alpha chain. In some embodiments, a heterologous antigen binding domain may be directly fused to the constant domain of a TCR beta chain.

### Recombinant Disulfide Bond

As summarized above, modified TCR chains of chimeric TCRs of the present disclosure may include one or more cysteine modifications. Such cysteine modifications, when paired between two chains having corresponding modifications may result in a recombinant disulfide bond between the paired chains.

In some embodiments, a chimeric TCR of the present disclosure may include a first cysteine modification in an alpha chain and a second cysteine modification in a beta chain where the first and second cysteine modifications, when both chains are present in a cell, form a recombinant disulfide bond between the alpha chain and the beta chain. Such cysteine modifications that form a recombinant disulfide bond may be referred to as “corresponding cysteine modifications”.

In some instances, a modified TCR alpha chain may include a substitution of a residue to a cysteine resulting in a cysteine modification sufficient to produce a recombinant disulfide bond. Any appropriate residue of a TCR alpha chain having a corresponding residue in a TCR beta chain that, when mutated to a cysteine results in a recombinant disulfide bond, may be employed in generating a cysteine modified alpha chain. In some instances, the substituted residue is a residue present in the TCR alpha constant region. In some instances, the substitution is a tyrosine to cysteine substitution. In some instances, the substitution is a T48C substitution, or corresponding mutation, such as the T48C substitution present in the following human TCR alpha chain constant region sequence: PNIQNPDPAVYQLRDSKSSDKSVCLFTDFDSQTNVSQSKDSDVYITDKCVLDMRSMDFKSNSAV AWSNKSDFACANAFNNSIIPEDTFFPSPESSCDVKLVEKSFETDTNLNFQNLSVIGFRILLLKVAG FNLLMTLRLWSS (SEQ ID NO:11). In some instances, the substitution is a T84C substitution, or corresponding mutation, such as the T84C substitution present in the following mouse TCR alpha chain constant region sequence:

(SEQ ID NO: 12)
PYIQNPEPAVYQLKDPRSQDSTLCLFTDFDSQINVPKTMESGTFITDKTV
LDMKAMDSKSNGAIAWSNQTSFTCQDIFKETNACYPSSDVPCDATLTEKS
FETDMNLNFQNLSVMGLRILLLKVAGFNLLMTLRLWSS.

In some instances, a subject TCR alpha chain or corresponding domain thereof (e.g., as present in a domain swapped chain), may have at least about 75%, at least about 80%, at least about 85%, at least about 90%, at least about 95%, at least about 98%, or 100% amino acid sequence identity to the T48C or T84C containing TCR alpha sequences provided above.

In some embodiments, a chimeric TCR of the present disclosure may include a first cysteine modification in a beta chain and a second cysteine modification in an alpha chain where the first and second cysteine modifications, when both chains are present in a cell, form a recombinant disulfide bond between the beta chain and the alpha chain. Such cysteine modifications that form a recombinant disulfide bond may be referred to as “corresponding cysteine modifications”.

In some instances, a modified TCR beta chain may include a substitution of a residue to a cysteine resulting in a cysteine modification sufficient to produce a recombinant disulfide bond. Any appropriate residue of a TCR beta chain having a corresponding residue in a TCR beta chain that, when mutated to a cysteine results in a recombinant disulfide bond, may be employed in generating a cysteine modified beta chain. In some instances, the substituted residue is a residue present in the TCR beta constant region. In some instances, the substitution is a serine to cysteine substitution. In some instances, the substitution is a S58C substitution, or corresponding mutation, such as the S58C substitution present in the following human TCR beta chain constant region sequence: EDLNKVFPPEVAVFEPSEAEISHTQKATLVCLATGFFPDHVELSWWVNGKEVHSGVCTDPQPLK EQPALNDSRYCLSSRLRVSATFWQNPRNHFRCQVQFYGLSENDEWTQDRAKPVTQIVSAEAWG RADCGFTSVSYQQGVLSATILYEILLGKATLYAVLVSALVLMAMVKRKDF (SEQ ID NO:13). In some instances, the substitution is a S79C substitution, or corresponding mutation, such as the S79C substitution present in the following mouse TCR beta chain constant region sequence:

(SEQ ID NO: 14)
EDLRNVTPPKVSLFEPSKAEIANKQKATLVCLARGFFPDHVELSWWVNGK
EVHSGVSTDPQAYKESNYSYCLSSRLRVCATFWHNPRNHFRCQVQFHGLS
EEDKWPEGSPKPVTQNISAEAWGRADCGITSASYQQGVLSATILYEILLG
KATLYAVLVSTLVVMAMVKRKNS.

In some instances, a subject TCR beta chain or corresponding domain thereof (e.g., as present in a domain swapped chain), may have at least about 75%, at least about 80%, at least about 85%, at least about 90%, at least about 95%, at least about 98%, or 100% amino acid sequence identity to the S58C or S79C containing TCR beta sequences provided above.

As will be obvious to those skilled in the art, mutation(s) in TCR chain sequence, including e.g., a chain sequence and/or TCR _{R} chain sequence, may be one or more of substitution(s), deletion(s) or insertion(s), including where mutations are introduced generally or for the specific purpose of introducing a cysteine modification. Mutations in TCR chains, or other polypeptides, can be carried out using any appropriate method including, but not limited to, those based on polymerase chain reaction (PCR), restriction enzyme-based cloning, or ligation independent cloning (LIC) procedures. These methods are detailed in many standard molecular biology texts, including but not limited to e.g., Sambrook & Russell, (2001) Molecular Cloning—A Laboratory Manual (3^{rd} Ed.) CSHL Press and Rashtchian, (1995) Curr Opin Biotechnol 6 (1): 30-6.

### Domain Swapped Chains

In some instances, a chimeric TCR may include one or more domain-swapped chains. By “domain-swapped chains” is generally meant TCR chains in which domains have been swapped between the a and βchains. When paired, domain-swapped TCRs assemble with CD3, express on the cell surface, and mediate antigen-specific T cell responses. Useful examples of domain-swapped chains include but are not limited to e.g., those described in Bethune et al. eLife 2016; 5:e19095; the disclosure of which is incorporated herein by reference in its entirety. In some instances, a chimeric TCR may include a domain-swapped alpha chain, a domain-swapped beta chain, and/or the like.

Domain swapped chains of a chimeric TCR may be domain swapped at any convenient and appropriate location. In some instances, a TCR chain may be domain swapped at the transmembrane domain resulting in a transmembrane domain swapped TCR chain. In some instances, a TCR chain may be domain swapped at one or more cytoplasmic regions resulting in a cytoplasmic region swapped TCR chain. In some instances, a TCR chain may be domain swapped at one or more chain connecting regions resulting in a chain connecting region swapped TCR chain.

In some instances, a domain swapped TCR alpha chain may include one or more domains of a TCR beta chain. For example, in some instances, a domain swapped TCR alpha chain may include a TCR beta chain connecting peptide domain, including e.g., a chain connecting peptide having 85% or greater (including 90% or great, 95% or greater, 99% or greater or 100%) sequence identity to the following TCR beta chain connecting peptide sequence: CGFTSVSYQQGVLSAT (SEQ ID NO:9). In some instances, a domain swapped TCR alpha chain may include a TCR beta chain transmembrane domain, including e.g., a transmembrane having 85% or greater (including 90% or great, 95% or greater, 99% or greater or 100%) sequence identity to the following TCR beta chain transmembrane domain sequence: ILLGKATLYAVLVSALVLMAM (SEQ ID NO:10).

In some instances, a domain swapped TCR beta chain may include one or more domains of a TCR alpha chain. For example, in some instances, a domain swapped TCR beta chain may include a TCR alpha chain connecting peptide domain, including e.g., a chain connecting peptide having 85% or greater (including 90% or great, 95% or greater, 99% or greater or 100%) sequence identity to the following TCR alpha chain connecting peptide sequence: CDVKLVEKSFETDTNLNFQN (SEQ ID NO:7). In some instances, a domain swapped TCR beta chain may include a TCR alpha chain transmembrane domain, including e.g., a transmembrane having 85% or greater (including 90% or great, 95% or greater, 99% or greater or 100%) sequence identity to the following TCR alpha chain transmembrane domain sequence: VIGFRILLLKVAGFNLLMTL (SEQ ID NO:8).

In some instances, a domain swapped chain of a chimeric TCR of the present disclosure may be a constant domain-connecting peptide swapped chain. For example, in some instances, a constant domain-connecting peptide swapped chain may include: a beta chain constant region fragment linked to an alpha chain constant region fragment containing an alpha chain connecting peptide and an alpha chain transmembrane domain. In some instances, a constant domain-connecting peptide swapped chain may include: an alpha chain constant region fragment linked to a beta chain constant region fragment containing a beta chain connecting peptide and a beta chain transmembrane domain.

Such constant domain-connecting peptide swapped chains may include assemblages of the following constant chain fragments:

TCR beta chain constant domain fragment:
(SEQ ID NO: 15)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRAD
TCR alpha constant domain, connecting peptide (CP)
and transmembrane (TM) domain fragment:
(SEQ ID NO: 16)
CDVKLVEKSFETDTNLNFQNLSVIGFRILLLKVAGFNLLMTLRLWSS
TCR alpha chain constant domain fragment:
(SEQ ID NO: 17)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESS
TCR beta constant domain, CP and TM domain
fragment:
(SEQ ID NO: 18)
CGFTSVSYQQGVLSATILYEILLGKATLYAVLVSALVLMAMVKRKDF.

In some embodiments, domain swapped chains having such fragments assembled into the following domain swapped chains, with or without attached variable domains and/or other modifications, may be employed:

TCR beta chain (constant domain fragment) + TCR
alpha chain (CP + TM fragment):
(SEQ ID NO: 19)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCDVKLVEKSFETDTNLNFQN
LSVIGFRILLLKVAGFNLLMTLRLWSS
TCR alpha chain (constant domain fragment) + TCR
beta chain (CP + TM fragment):
(SEQ ID NO: 20)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCGFTS
VSYQQGVLSATILYEILLGKATLYAVLVSALVLMAMVKRKDF.

In some instances, a domain swapped chain of a chimeric TCR of the present disclosure may be a connecting peptide-transmembrane domain swapped chain. For example, in some instances, a connecting peptide-transmembrane domain swapped chain may include: a beta chain constant region fragment linked to an alpha chain constant region fragment containing an alpha chain transmembrane domain. In some instances, a connecting peptide-transmembrane domain swapped chain may include: an alpha chain constant region fragment linked to a beta chain constant region fragment containing a beta chain transmembrane domain.

Such connecting peptide-transmembrane domain swapped chains may include assemblages of the following constant chain fragments:

TCR beta chain constant domain fragment:
(SEQ ID NO: 21)
EDLNKVFPPEVAVFEPSEAEISHTQKATLVCLATGFFPDHVELSWWVNGK
EVHSGVSTDPQPLKEQALNDSRYCLSSRLRVSATFWQNPRNHFRCQVQFY
GLSENDEWTQDRAKPVTQIVSAEAWGRADCGFTSVSYQQGVLSAT
TCR alpha chain constant domain TM containing
fragment:
(SEQ ID NO: 85)
NLSVIGFRILLLKVAGFNLLMTLRLWSS
TCR alpha chain constant domain fragment:
(SEQ ID NO: 22)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQ
TCR beta chain constant domain TM containing
fragment:
(SEQ ID NO: 23)
ILYEILLGKATLYAVLVSALVLMAMVKRKDF.

In some embodiments, domain swapped chains having such fragments assembled into the following domain swapped chains, with or without attached variable domains and/or other modifications, may be employed:

TCR beta chain constant region fragment + TCR
alpha chain constant domain TM containing fragment:
(SEQ ID NO: 24)
EDLNKVFPPEVAVFEPSEAEISHTQKATLVCLATGFFPDHVELSWWVNGK
EVHSGVSTDPQPLKEQALNDSRYCLSSRLRVSATFWQNPRNHFRCQVQFY
GLSENDEWTQDRAKPVTQIVSAEAWGRADCGFTSVSYQQGVLSATNLSVI
GFRILLLKVAGFNLLMTLRLWSS
TCR alpha chain constant region fragment + TCR
beta chain constant domain TM containing fragment:
(SEQ ID NO: 25)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQILYEILLGKATLYAVLVSALVLMAMVKRKDF.

The above examples of domain swapped alpha and beta chains are provided as non-limiting examples and the instant disclosure is not intended to be limited to only those examples of domain swapped chains specifically disclosed. On the contrary, swapping of domains between alpha and beta TCR chains may be readily performed and various other swapped chains may be generated.

### Co-Stimulatory Domains

As noted above, in some instances, a chimeric TCR may include a co-stimulatory domain, including e.g., a co-stimulatory domain that is heterologous to one or more TCR chains, including e.g., heterologous to the TCR alpha chain, heterologous to the TCR beta chain, etc. The co-stimulatory domain, when present on (e.g., fused to) one or more chains of a TCR will generally be intracellular. A subject chimeric TCR may include any number of co-stimulatory domains including but not limited to e.g., one, two, three, four, five, six, seven, eight, nine, ten or more. In some instances, all co-stimulatory domains of a chimeric TCR will be present on one chain, including e.g., where all co-stimulatory domains are fused to the TCR alpha chain or where all co-stimulatory domains are fused to the TCR beta chain. In some instances, both the alpha and beta chains of a chimeric TCR may include at least one co-stimulatory domain, including where the alpha and beta chains have the same number of co-stimulatory domains or where the alpha and beta chains have different numbers of co-stimulatory domains. In some instances, the alpha and beta chains may include the same co-stimulatory domain. In some instances, the alpha and beta chains may not include the same co-stimulatory domain (i.e., the alpha and beta chains may include different co-stimulatory domains).

A co-stimulatory domain suitable for use in a subject chimeric TCR may be any functional unit of a polypeptide as short as a 3 amino acid linear motif and as long as an entire protein, where size of the co-stimulatory domain is restricted only in that the domain must be sufficiently large as to retain its function and sufficiently small so as to be compatible with the other components of the chimeric TCR or the chosen mode of expression/delivery. Accordingly, a co-stimulatory domain may range in size from 3 amino acids in length to 1000 amino acids or more and, in some instances, can have a length of from about 30 amino acids to about 70 amino acids (aa), e.g., a stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, stimulatory domain can have a length of from about 70 aa to about 100 aa, from about 100 aa to about 200 aa, or greater than 200 aa.

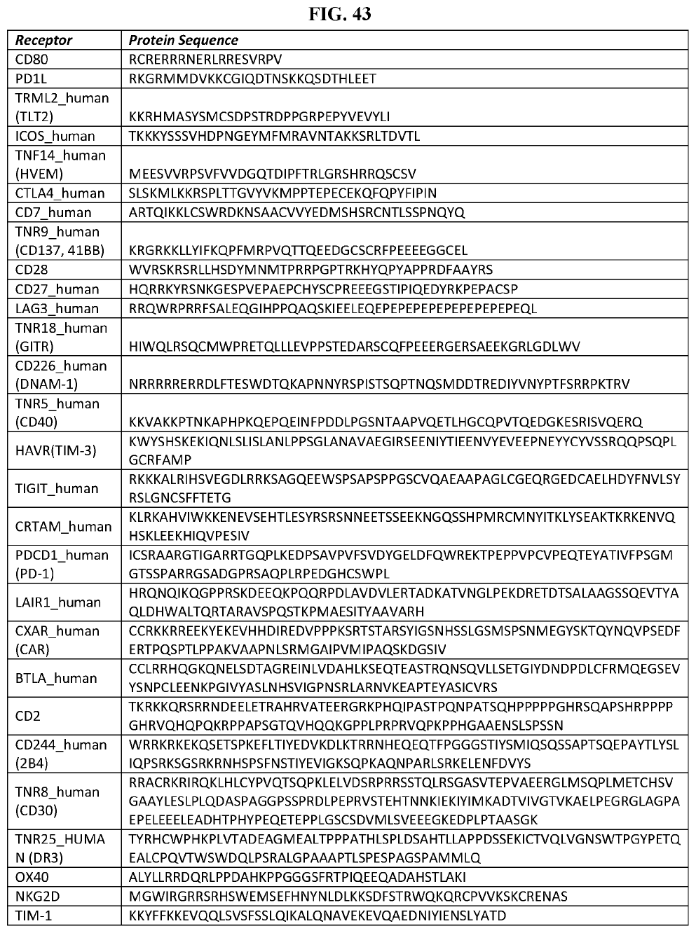

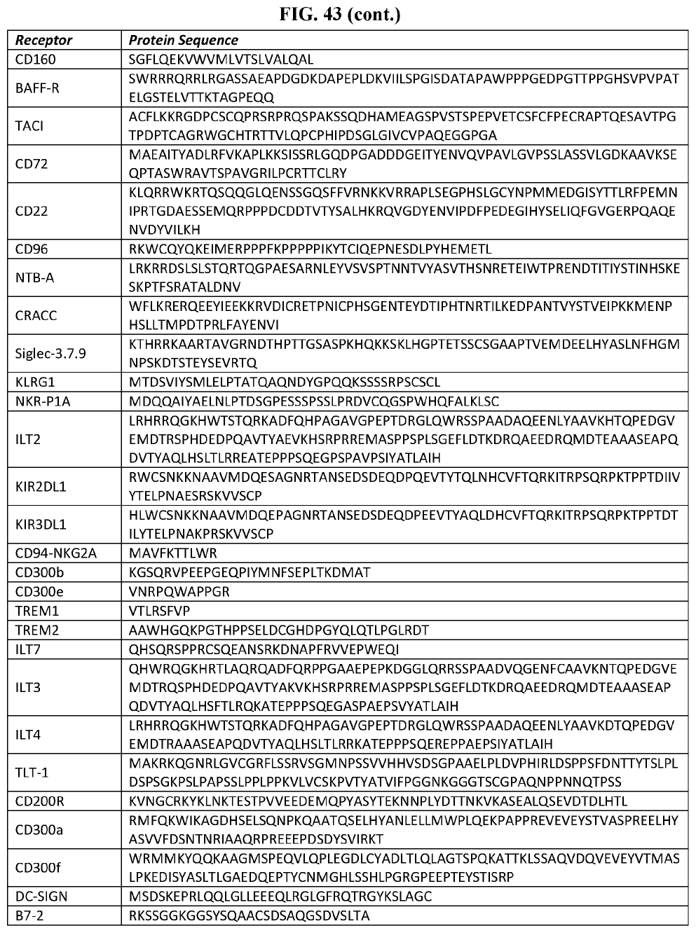

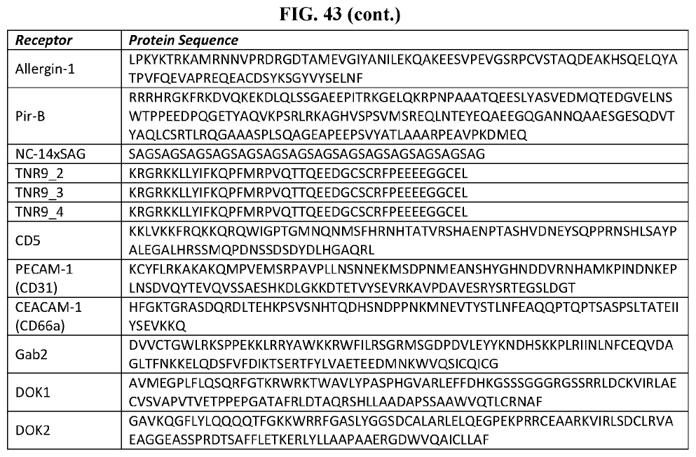

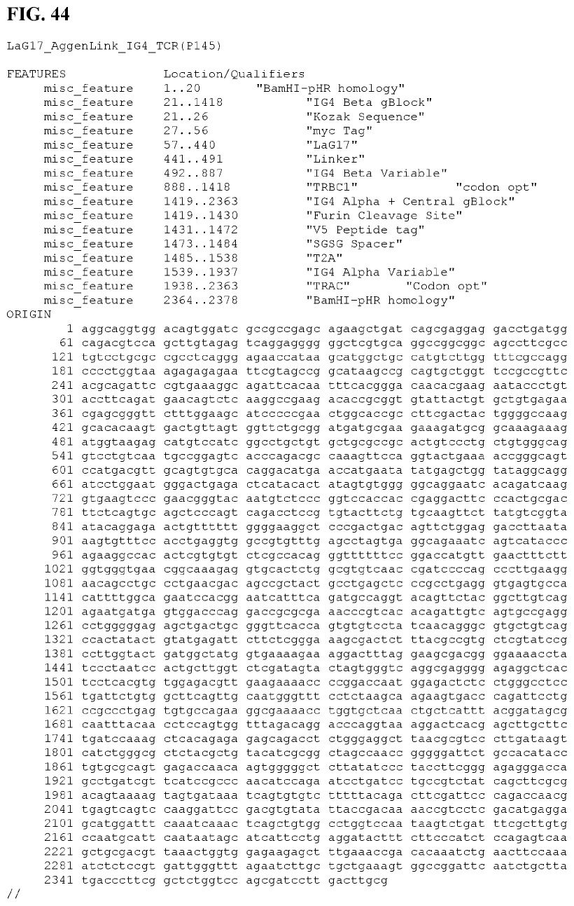

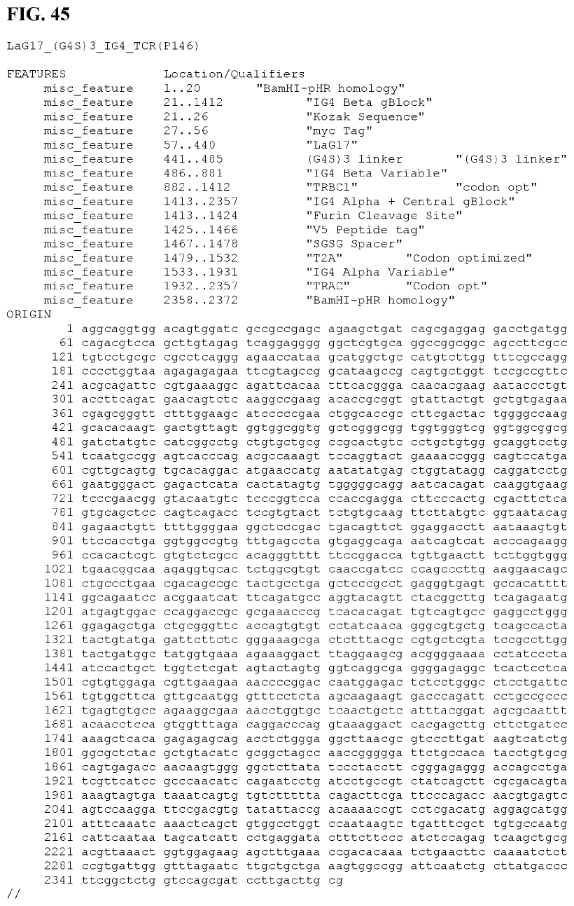

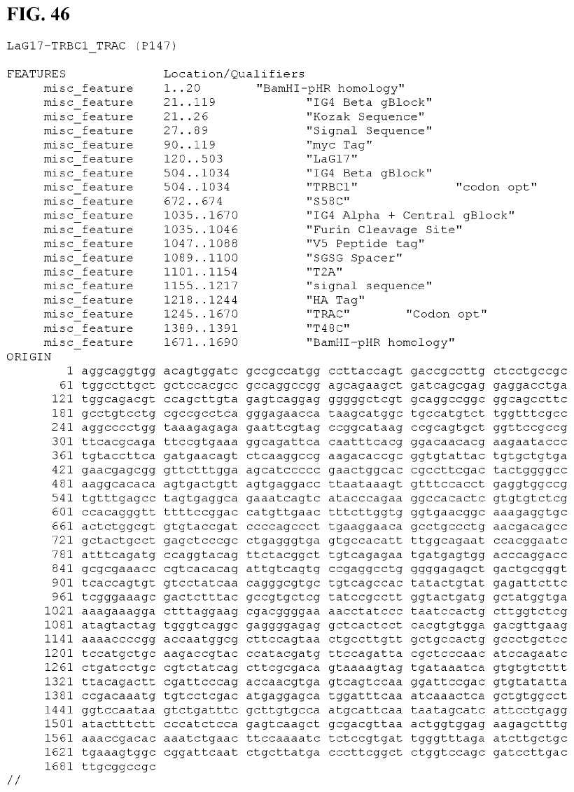

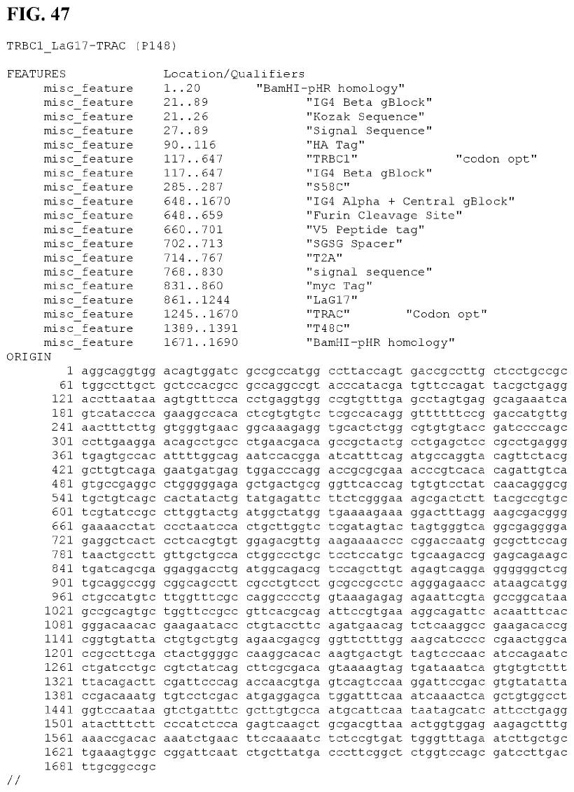

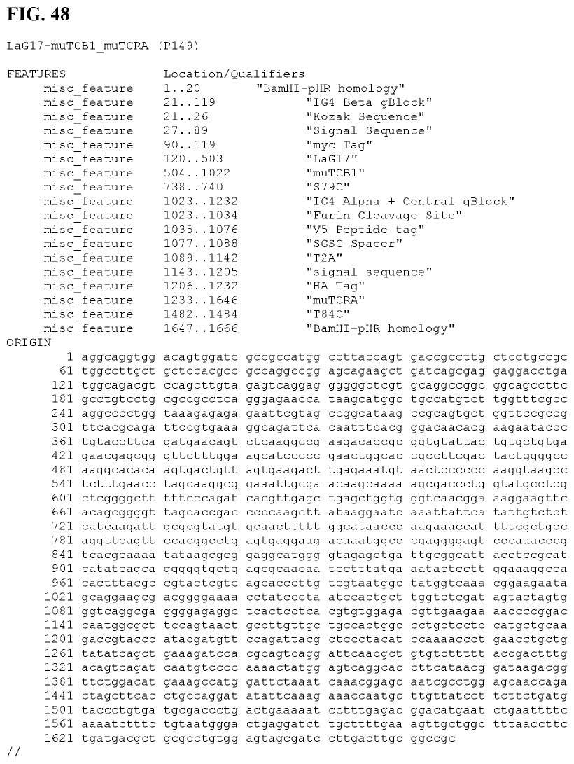

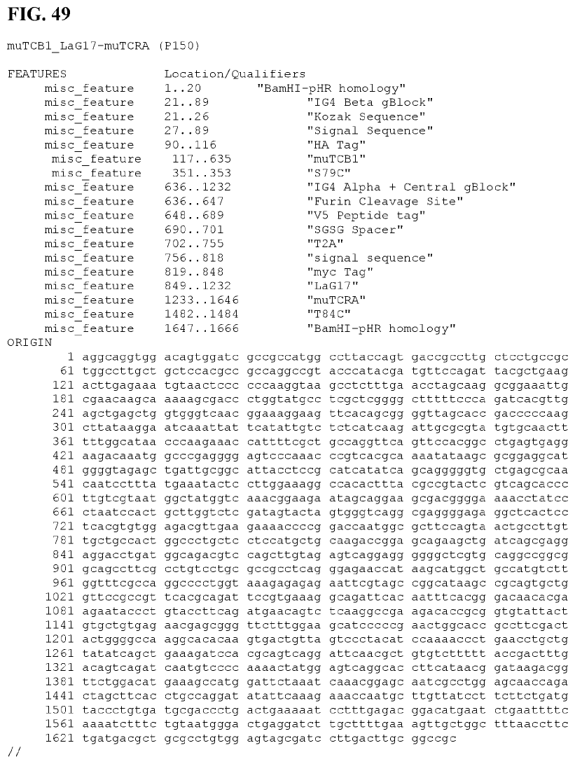

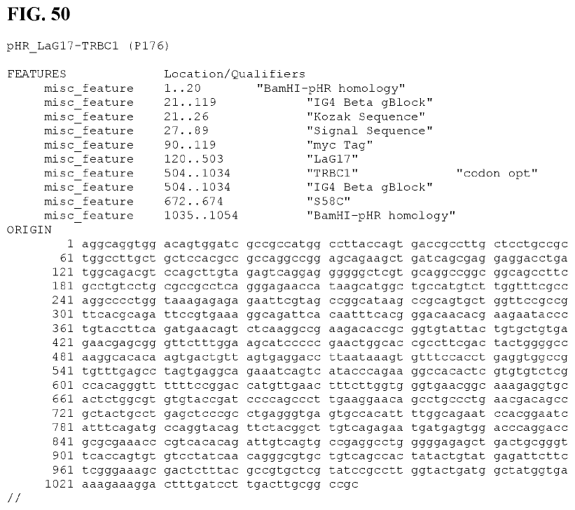

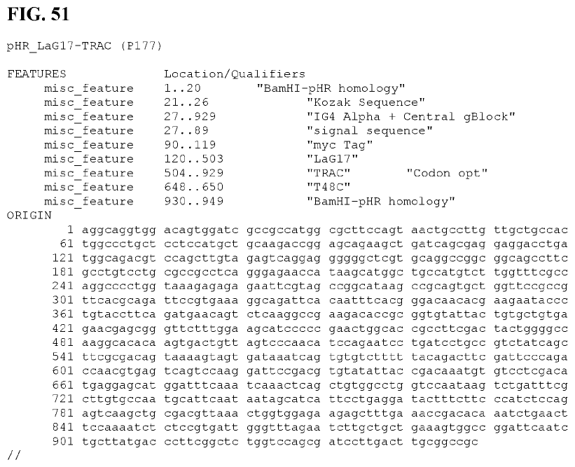

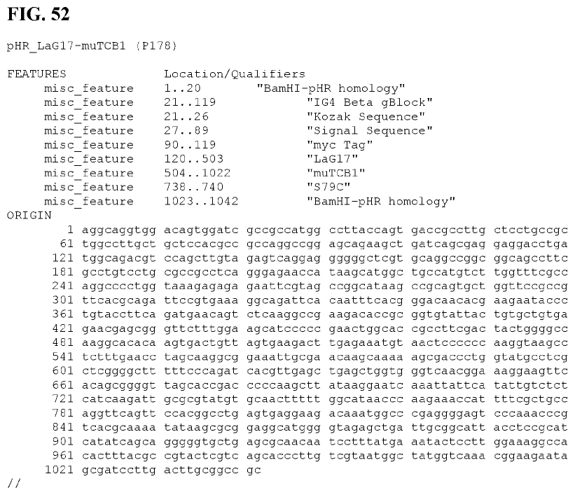

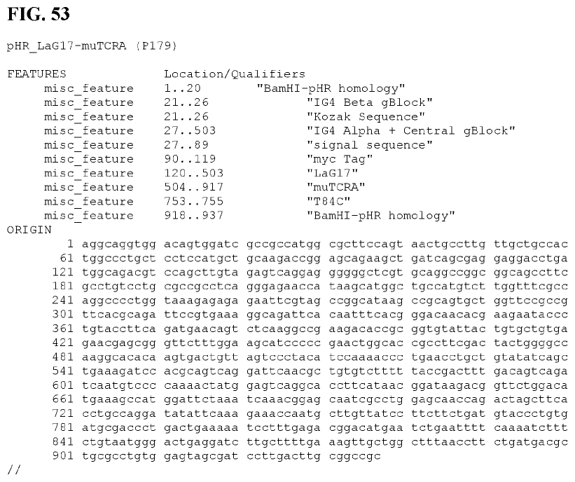

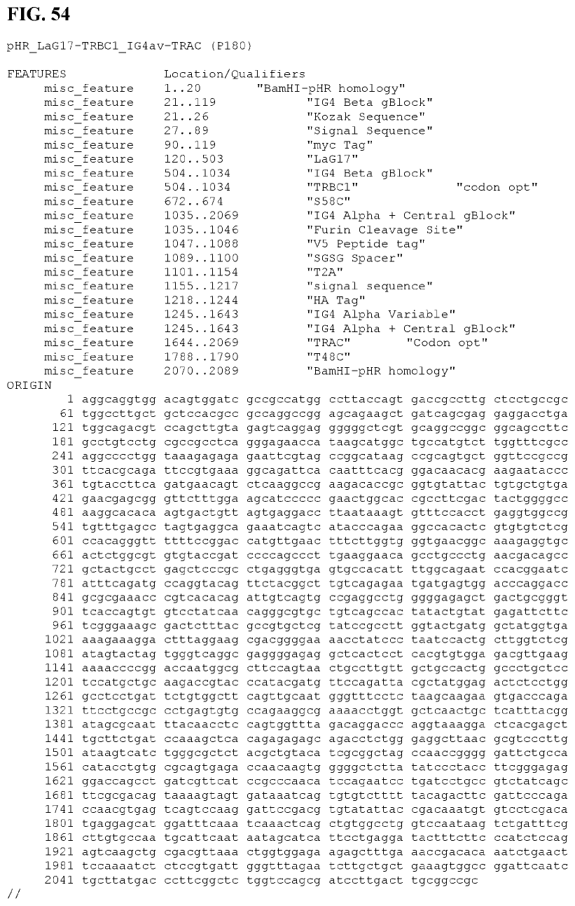

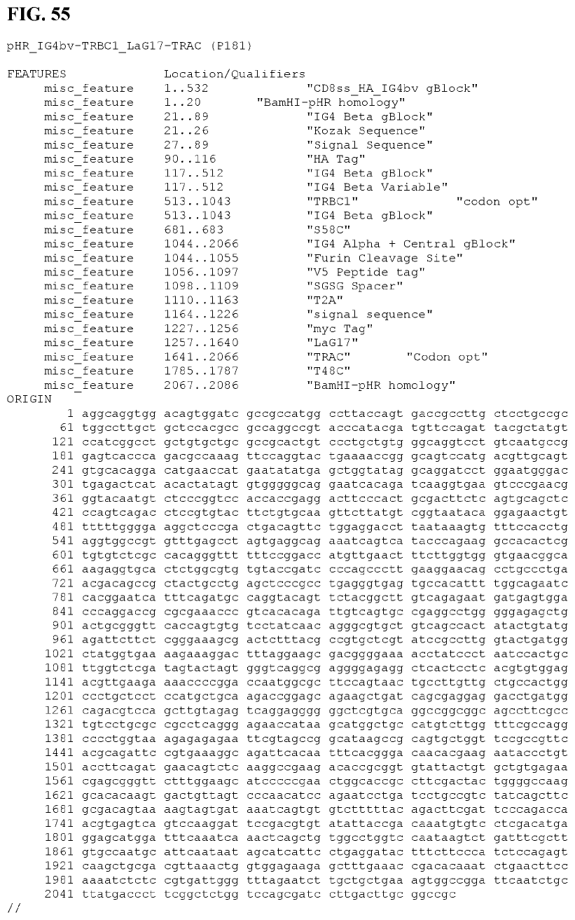

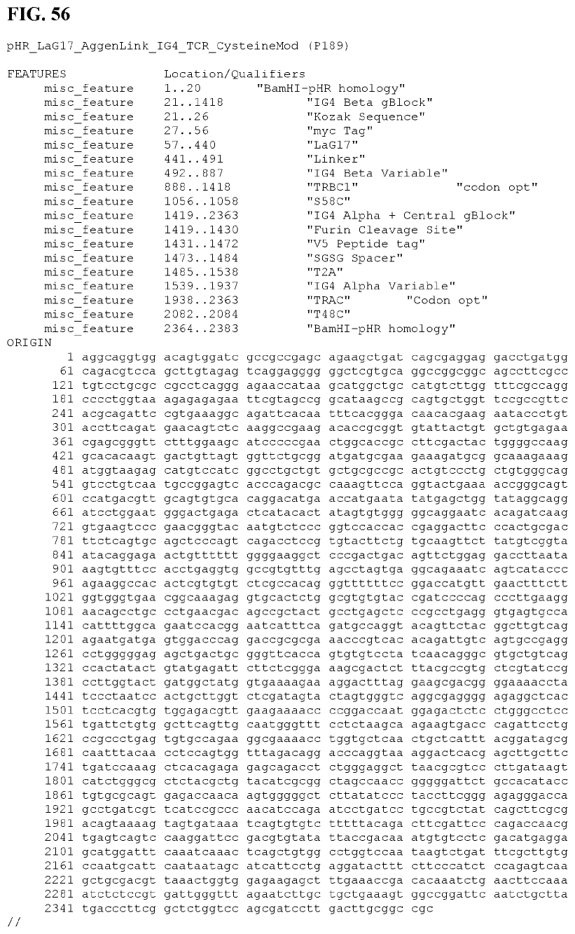

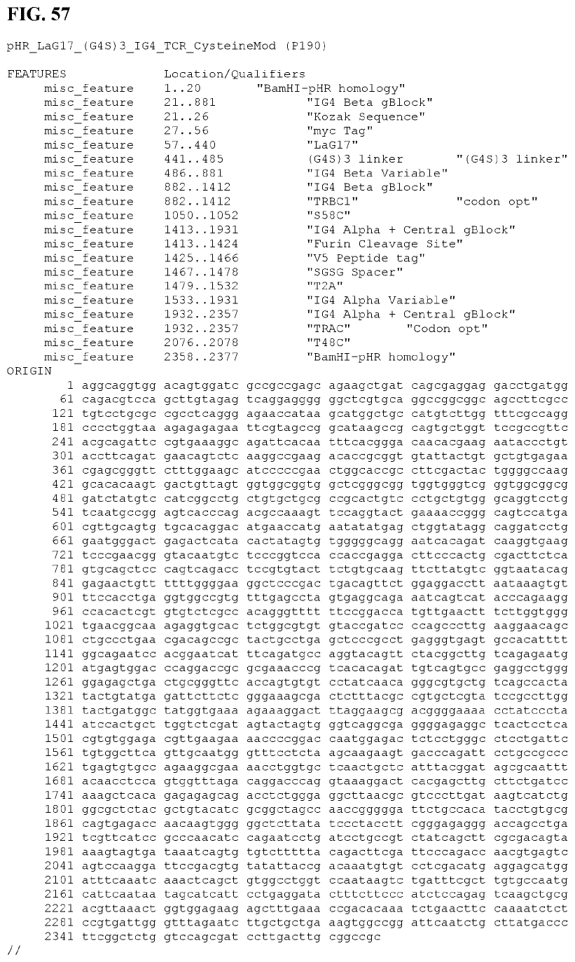

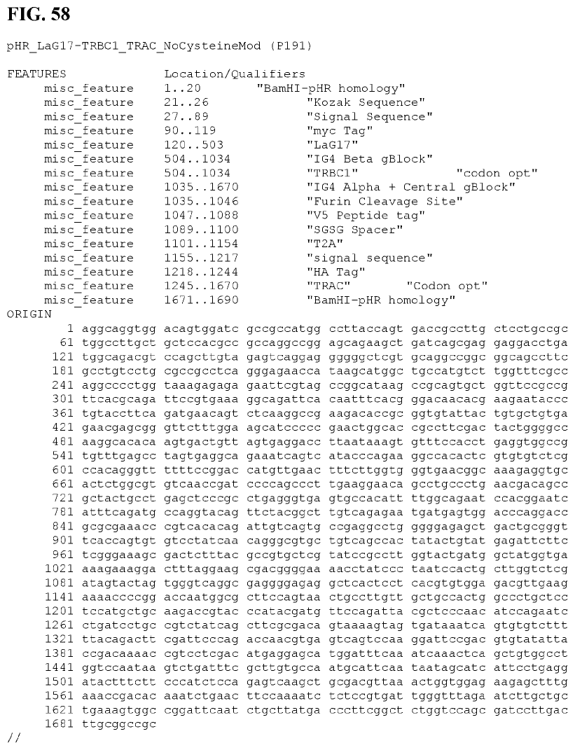

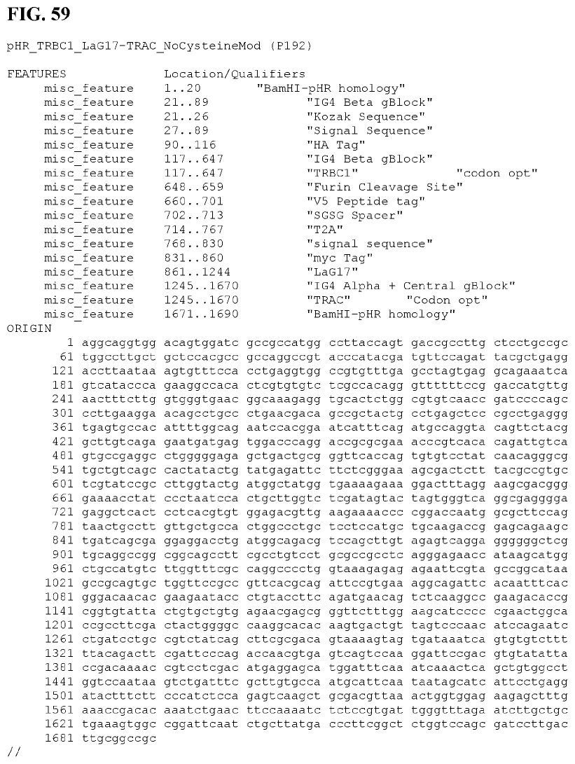

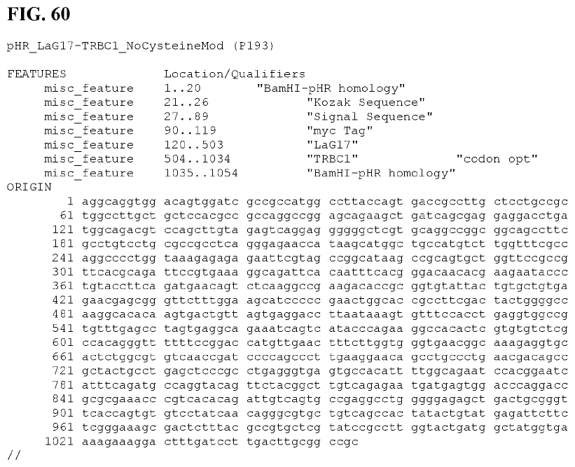

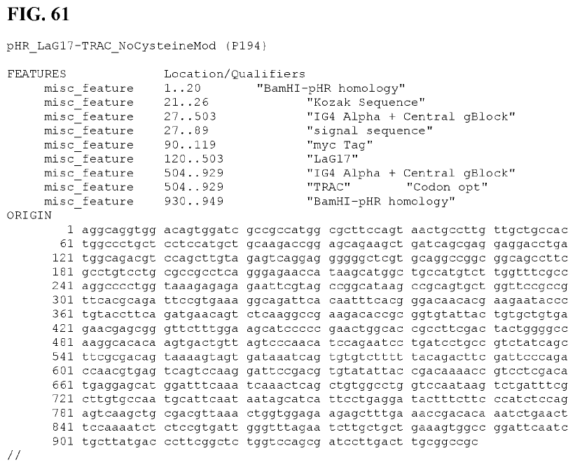

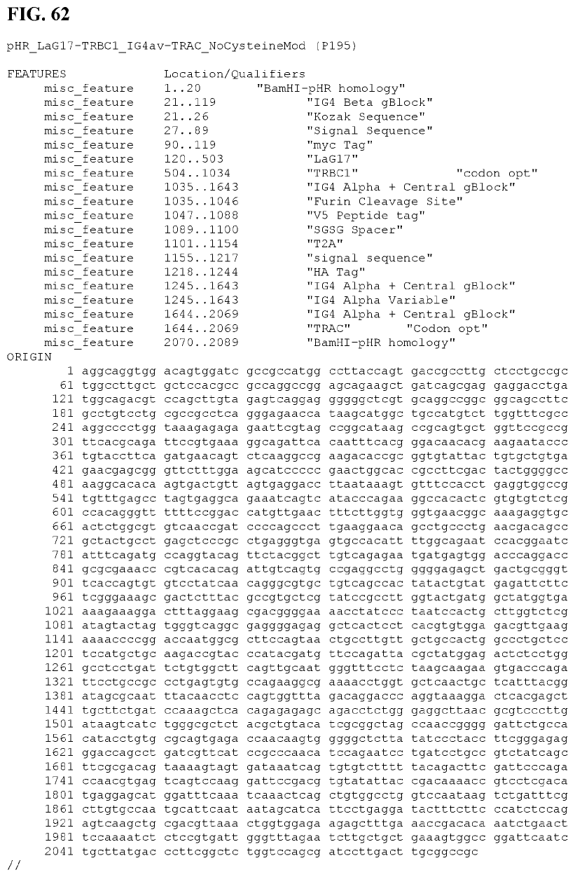

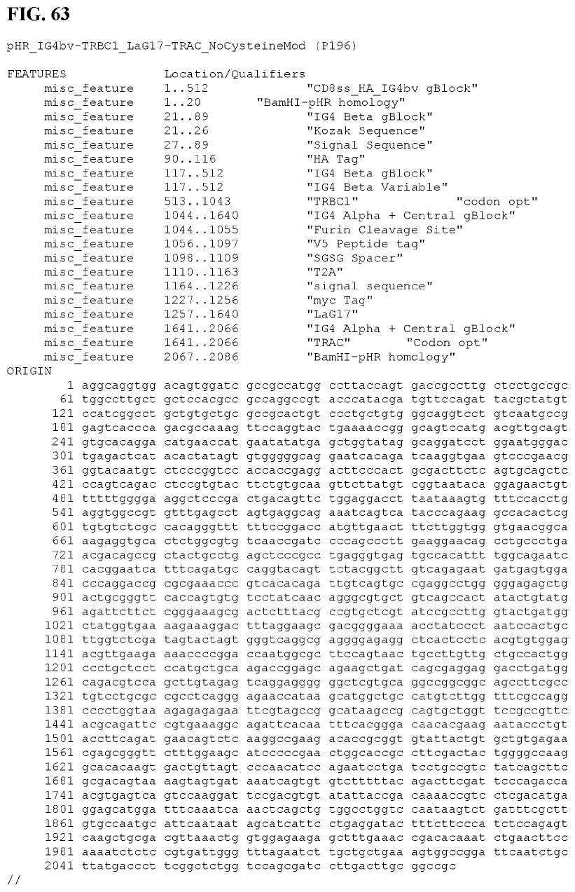

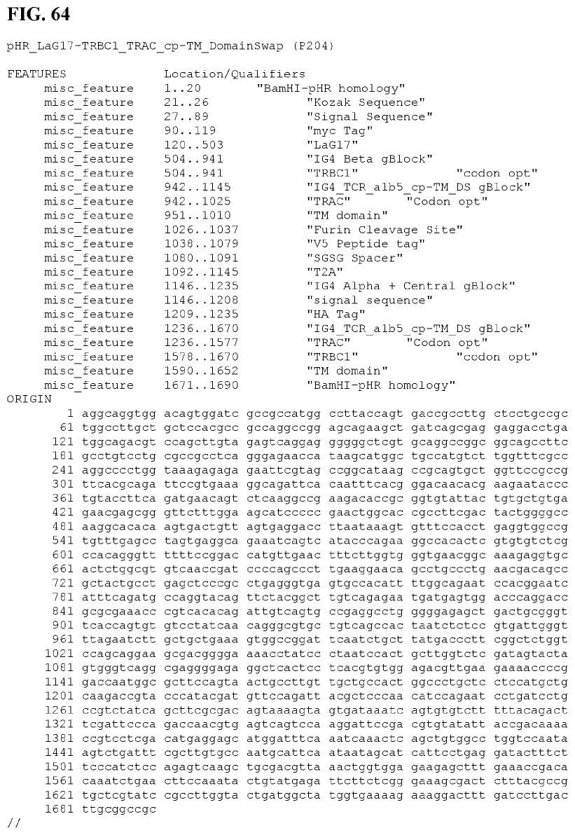

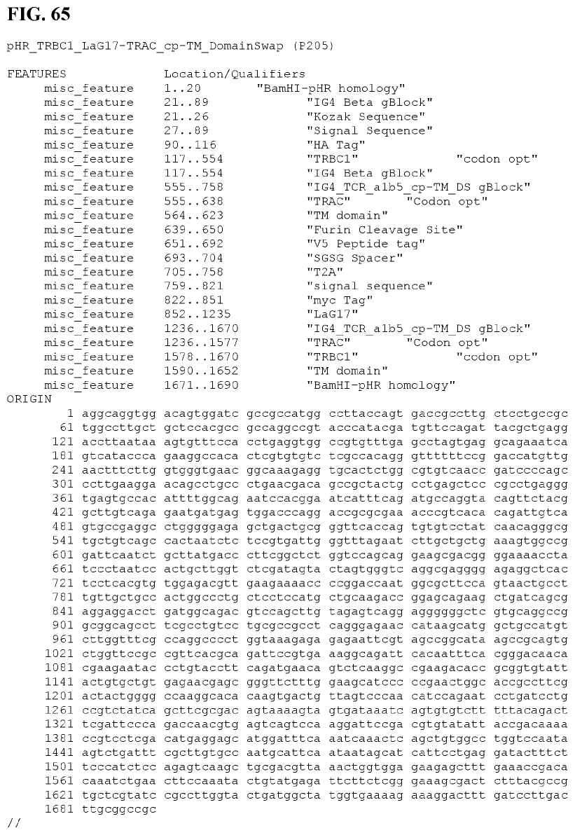

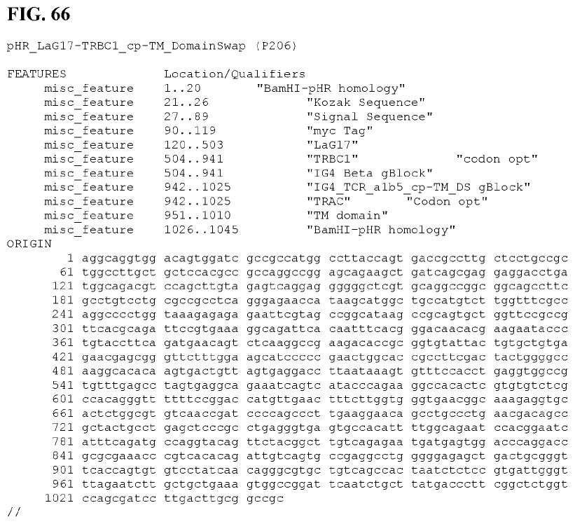

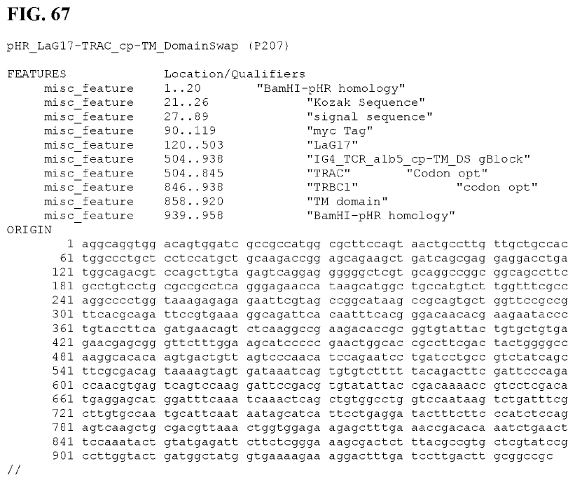

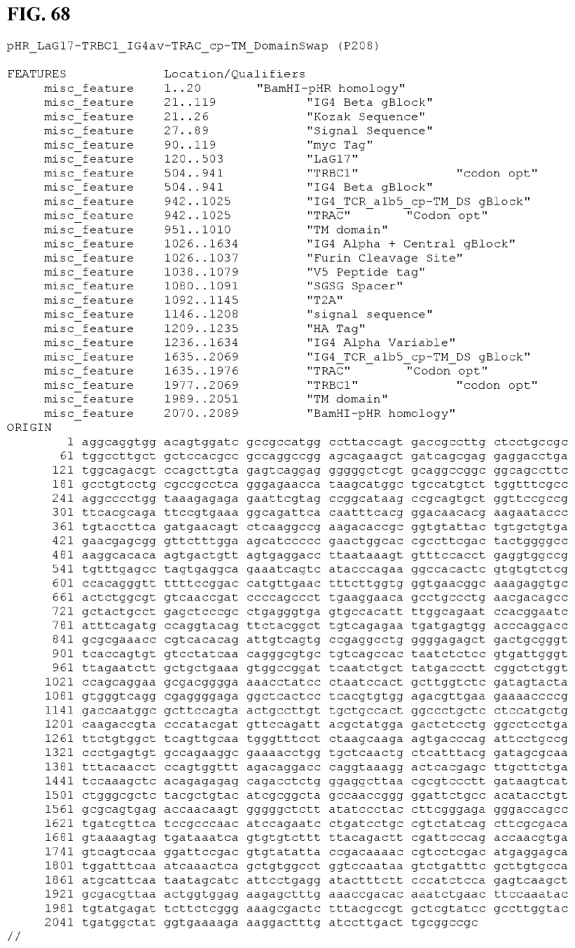

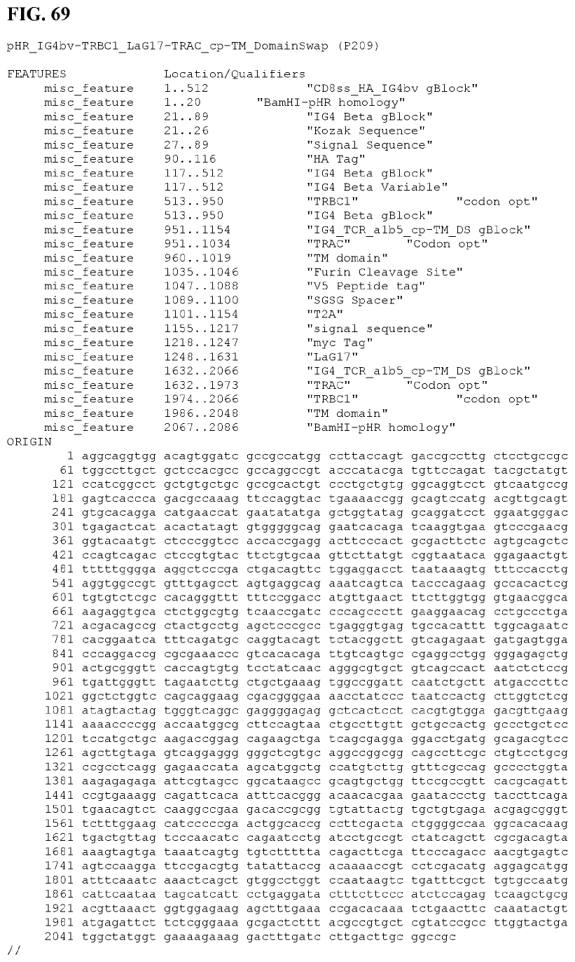

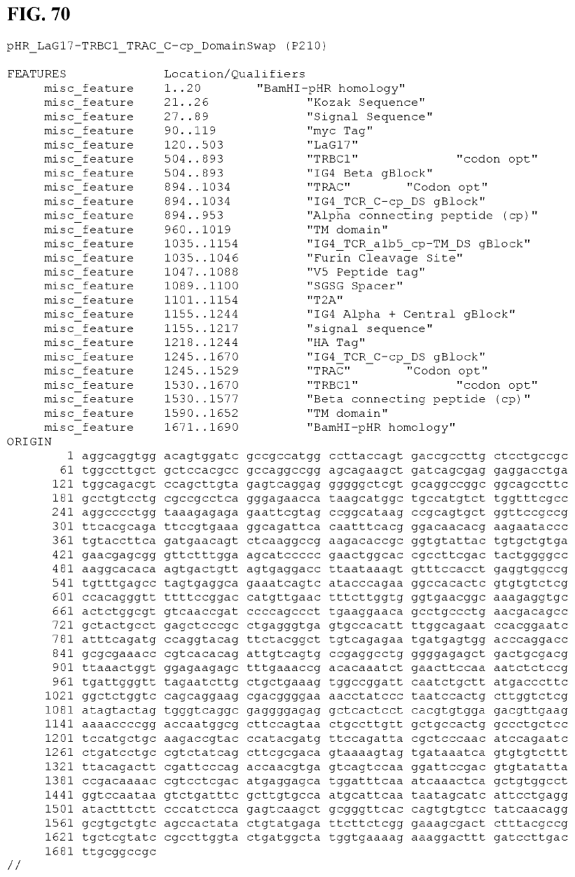

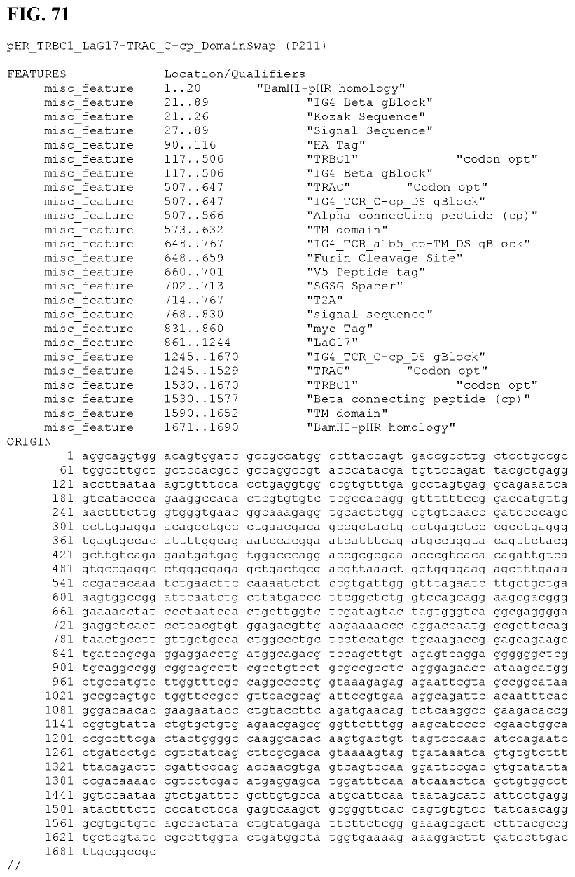

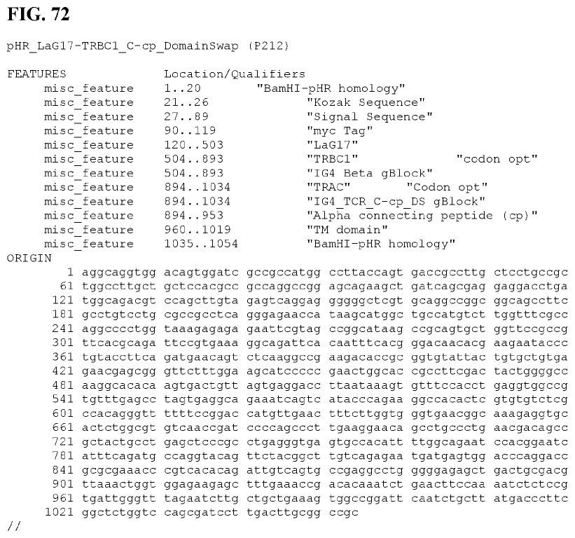

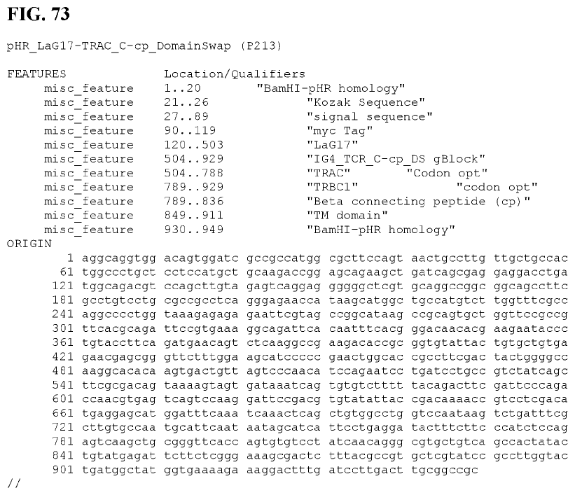

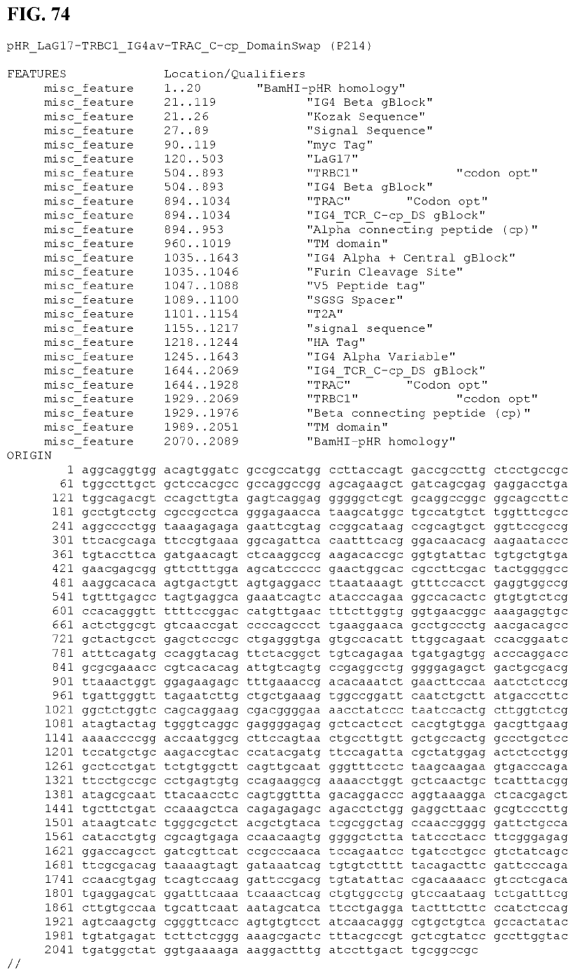

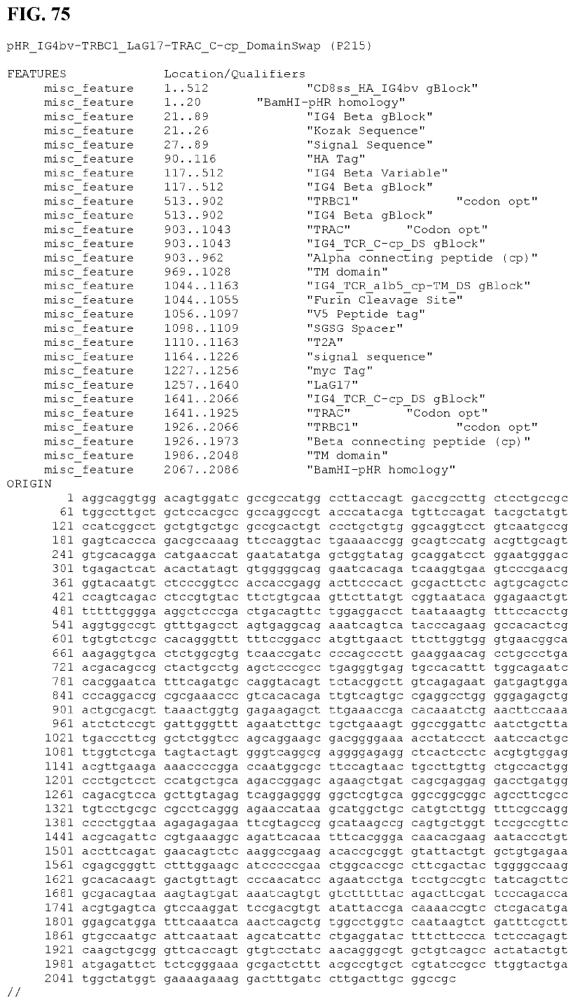

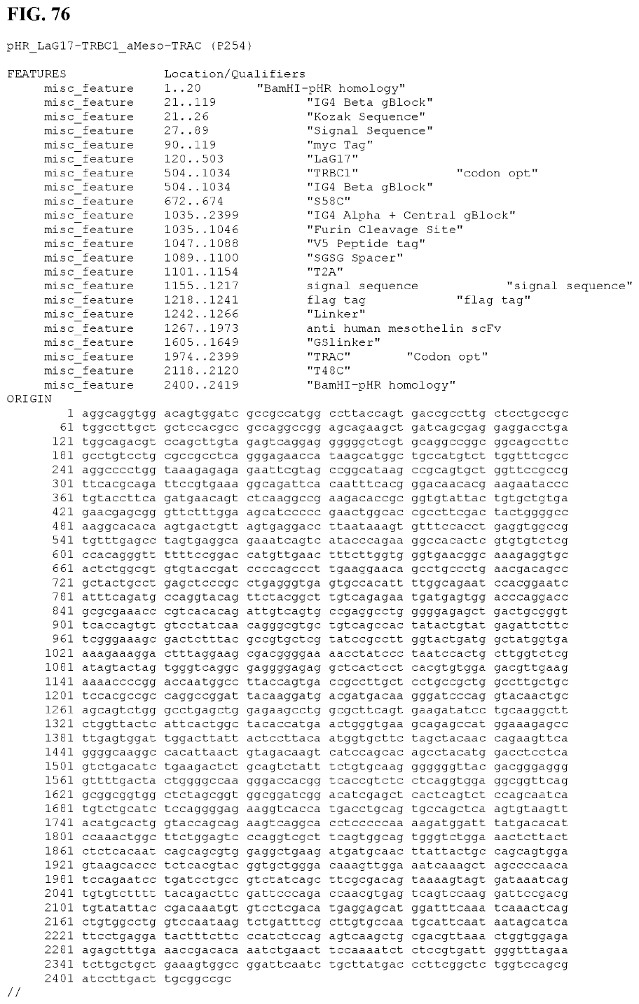

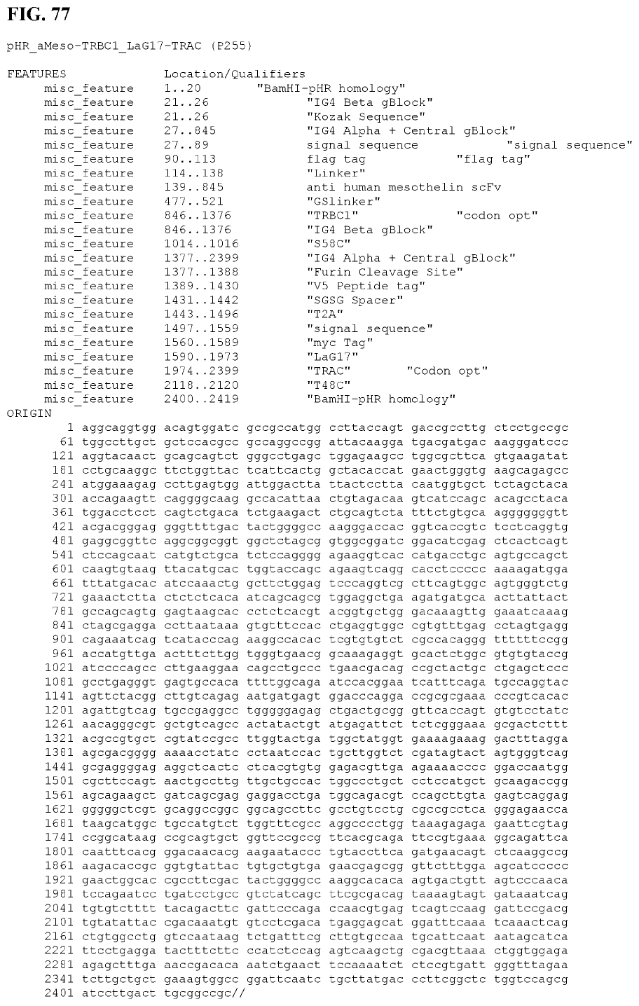

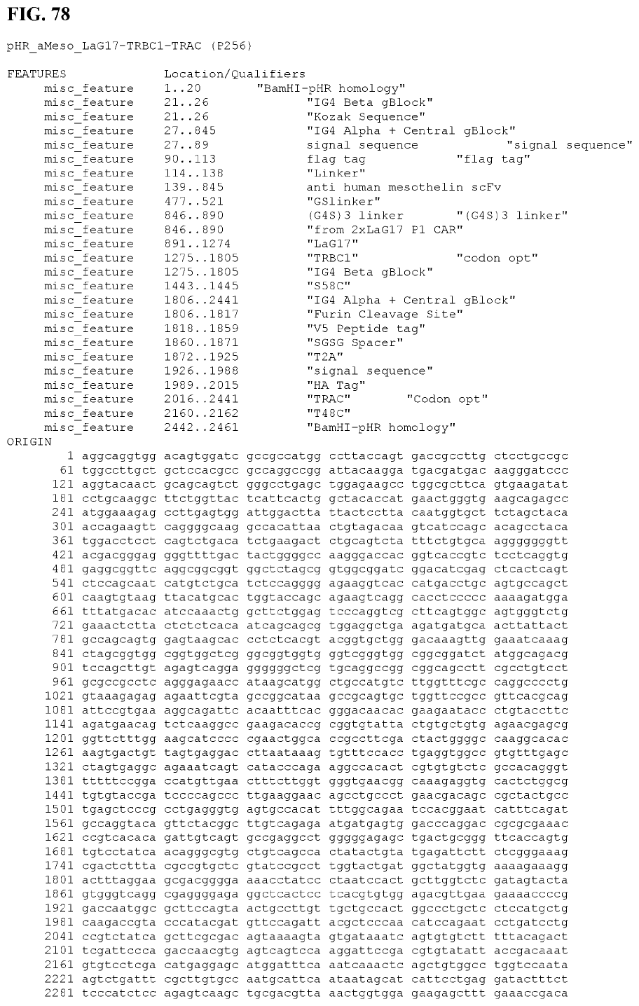

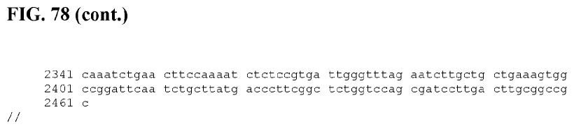

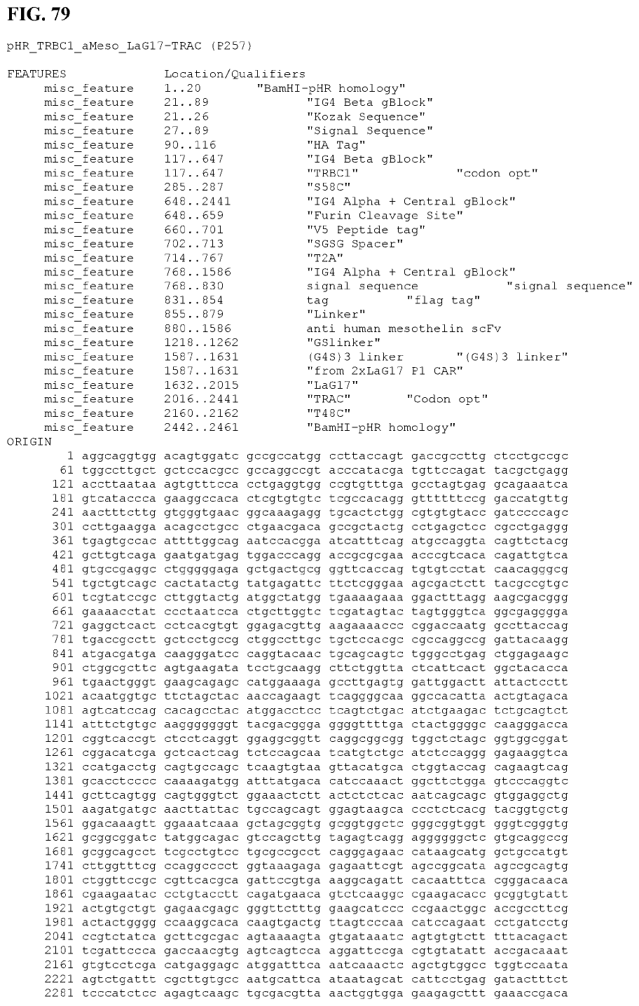

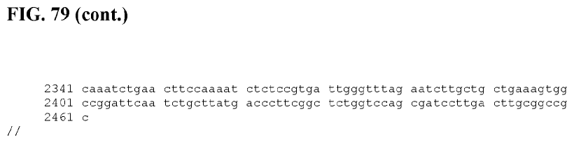

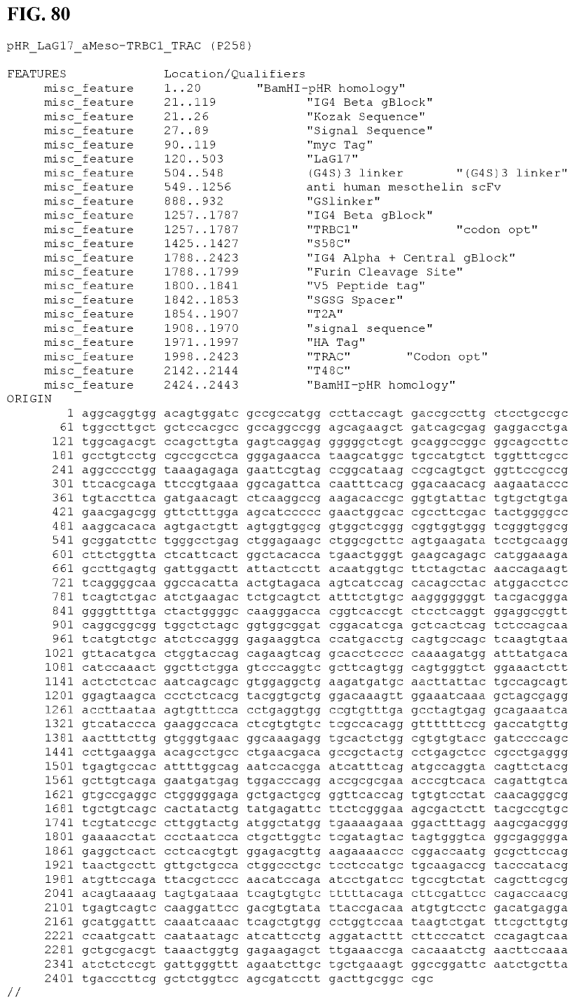

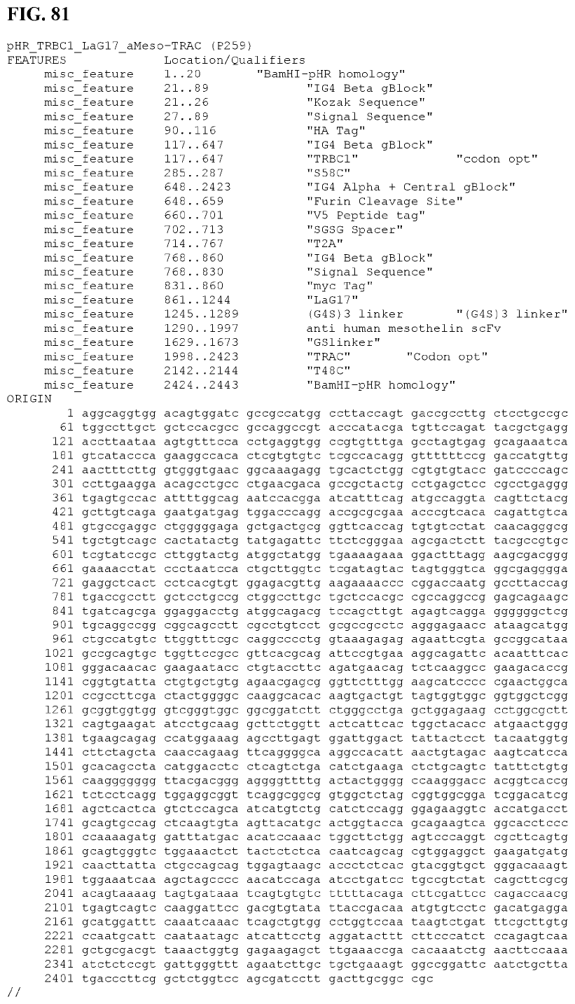

Co-stimulation, as it relates to co-stimulatory domains, generally refers to a secondary non-specific activation mechanism through which a primary specific stimulation is propagated. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. Nat Rev Immunol (2013) 13(4):227-42, the disclosure of which is incorporated herein by reference in its entirety. Co-stimulatory domains are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). Non-limiting examples of suitable co-stimulatory polypeptides include, but are not limited to, 4-1BB (CD137), CD28, ICOS, OX-40, BTLA, CD27, CD30, GITR, and HVEM. In some instances, a co-stimulatory domain, e.g., as used in a chimeric TCR of the instant disclosure may include a co-stimulatory domain listed in Table 1 (provided in FIG. 43). In some instances, a co-stimulatory domain of a chimeric TCR comprises a an amino acid sequence having at least about 75%, at least about 80%, at least about 85%, at least about 90%, at least about 95%, at least about 98%, or 100% amino acid sequence identity to a co-stimulatory domain as described herein.

In some instances, a chimeric TCR may contain a co-stimulatory domain, derived from an intracellular portion of a transmembrane protein listed in Table 1. For example, a suitable co-stimulatory domain can comprise an amino acid sequence having at least about 75%, at least about 80%, at least about 85%, at least about 90%, at least about 95%, at least about 98%, or 100% amino acid sequence identity to an amino acid sequence listed in Table 1. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165, aa from about 165 aa to about 170 aa, from about 170 aa to about 175 aa, from about 175 aa to about 180 aa, from about 180 aa to about 185 aa, or from about 185 aa to about 190 aa.

As noted above, in some cases, a chimeric TCR may contain two more co-stimulatory domains, present on the same or different polypeptides. In some instances, where the chimeric TCR contains two more stimulatory domains, the stimulatory domains may have substantially the same amino acid sequences. For example, in some cases, the first stimulatory domain comprises an amino acid sequence that is at least about 90%, at least about 95%, at least about 98%, at least about 99%, or 100%, identical to the amino acid sequence of the second stimulatory domain In some instances, where the chimeric TCR contains two more stimulatory domains, the stimulatory domains of the subject chimeric TCR can have substantially the same length; e.g., the first and second stimulatory domains can differ in length from one another by fewer than 10 amino acids, or fewer than 5 amino acids. In some instances, where the chimeric TCR contains two more stimulatory domains, the first and second stimulatory domains have the same length. In some instances, where chimeric TCR contains two more stimulatory domains, the two stimulatory domains are the same.

Specific co-stimulatory domains, and the sequences thereof, that may find use in the subject chimeric TCRs include those that have been previously described and utilized in various contexts including but not limited to the contexts of engineered TCRs and chimeric antigen receptors (CARs), including e.g., those described in U.S. Patent Application Publication No. US 2015-0368342 A1; PCT Publication No. WO 2014/127261 and PCT Application Nos. US2016/049745; US2016/062612; the disclosures of which are incorporated herein by reference in their entirety.

### Single Chain Chimeric TCRs

A summarized above, in some instances, chains of a chimeric TCR of the present disclosure may be joined into a single chain, e.g., through the use of linking peptides. For example, in some instances, two modified TCR chains, e.g., a modified TCR alpha chain and a modified TCR beta chain may be linked by a linking peptide into a single chain chimeric TCR Linking of chains of a chimeric TCR may include a linking peptide having one or more transmembrane domains, facilitating the passage of the linking peptide through the cell membrane and allowing for the linkage of the intracellular end of a first chain to the extracellular end of a second chain.

Any suitable transmembrane domain may be employed in constructing a single chain chimeric TCR. In some instances, suitable transmembrane domains will include a transmembrane (TM) domain that provides for insertion of a polypeptide into the cell membrane of a eukaryotic (e.g., mammalian) cell.

In some instances, a TM domain employed in a single chain chimeric TCR may be an immune molecule TM domain, i.e., a TM domain derived from a molecule associated with an immune cell and/or an immune function or immune signaling of a cell. Non-limiting example of suitable immune cell transmembrane domains include but are not limited to e.g., a CD8 alpha derived TM, such as e.g., IYIWAPLAGTCGVLLLSLVITLYC (SEQ ID NO:26); a CD8 beta derived TM, such as e.g., LGLLVAGVLVLLVSLGVAIHLCC (SEQ ID NO:27); a CD4 derived TM, such as e.g., ALIVLGGVAGLLLFIGLGIFFCVRC (SEQ ID NO:28); a CD3 zeta derived TM, such as e.g., LCYLLDGILFIYGVILTALFLRV (SEQ ID NO:29); a CD28 derived TM, such as e.g., WVLVVVGGVLACYSLLVTVAFIIFWV (SEQ ID NO:30); a CD134 (0X40) derived TM, such as e.g., VAAILGLGLVLGLLGPLAILLALYLL (SEQ ID NO:31); a CD7 derived TM, such as e.g., ALPAALAVISFLLGLGLGVACVLA (SEQ ID NO:32); and variants thereof including e.g., those having at least 75%, at least 80%, at least 85%, at least, 90% at least, 95%, at least 96%, at least 97%, at least 98% or at least 99% sequence identity with any one of the described TM domain amino acid sequences.

In some embodiments, a single chain chimeric TCR, as described herein, may include, in order: a heterologous antigen binding domain linked to a TCR alpha chain extracellular domain, the intracellular domain of the TCR alpha chain linked to a linking polypeptide that includes a transmembrane domain, the linking polypeptide linked to an extracellular domain of a TCR beta chain. In such embodiments, the extracellular domains of the TCR alpha and/or beta chains may or may not include a variable domain (e.g., the alpha chain, the beta chain or both the alpha chain and the beta chain may be truncated).

In some embodiments, a single chain chimeric TCR, as described herein, may include, in order: a heterologous antigen binding domain linked to a TCR beta chain extracellular domain, the intracellular domain of the TCR beta chain linked to a linking polypeptide that includes a transmembrane domain, the linking polypeptide linked to an extracellular domain of a TCR alpha chain. In such embodiments, the extracellular domains of the TCR alpha and/or beta chains may or may not include a variable domain (e.g., the alpha chain, the beta chain or both the alpha chain and the beta chain may be truncated).

As described above, linking of domains (e.g., between domains of TCR chains, between a domain of a TCR chain and a heterologous domain, etc.) may be achieved with or without the use of polypeptide linkers. In some instances, with the exception of the linking polypeptide, which may be considered a “linker”, a single chain chimeric TCR may not include linkers between joined domains. In some instances, a single chain chimeric TCR may include one or more linkers between domains of the single chain chimeric TCR, including e.g., where linkers are employed as discussed above between domains of TCR chains, between a domain of a TCR chain and a heterologous domain, and the like. In some instances, the linking polypeptide may be joined to a domain of a single chain chimeric TCR through the use of a linker. In other instances, the linking polypeptide may be joined directly to a domain of a single chain chimeric TCR, without the use of an additional linker.

### Linkers

In some cases, a subject chimeric TCR includes a linker between any two adjacent domains or artificially (e.g., heterologously) linked chains. For example, in some instances, a linker can be disposed between a heterologous antigen binding domain and a variable domain of a TCR chain, e.g., a TCR alpha chain or a TCR beta chain. In some instances, a linker can be disposed between a heterologous antigen binding domain and a constant domain of a TCR chain, e.g., a TCR alpha chain or a TCR beta chain. In some instances, a linker can be disposed between two heterologous antigen binding domains, e.g., where a single chain includes two or more heterologous antigen binding domains. In some instances, a linker can be disposed between a heterologous signaling-related domain (e.g., a co-stimulatory domain) and an intracellular domain of a TCR chain, e.g., a TCR alpha chain or a TCR beta chain. Alternatively, in some instances, such junctions may be made directly, i.e., without the use of a linker.

Linkers may be utilized in a suitable configuration in a chimeric TCR provided they do not abolish the primary activities of the chimeric TCR including, e.g., the ability of the chimeric TCR to activate an immune cell, the ability of the antigen binding domain to bind its cognate antigen, etc.

Any suitable linker, including two or more linkers (e.g., where the two or more linkers are the same or different and including where the multiple linkers are three or more, four or more, five or more, six or more, etc. and including where all the linkers are different and where the multiple linkers include an mix of some linkers utilized in more than one location and some linkers utilized specifically in only one location and the like) may be utilized in the subject chimeric TCRs.

A linker peptide may have any of a variety of amino acid sequences. Proteins can be joined by a spacer peptide, generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 40 amino acids, from 1 amino acid to 35 amino acids, from 1 amino acid to 30 amino acids, from 1 amino acid to 25 amino acids, from 1 amino acid to 20 amino acids, from 5 amino acids to 35 amino acids, from 5 amino acids to 30 amino acids, from 10 amino acids to 35 amino acids, from 15 amino acids to 30 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ (SEQ ID NO:33) and (GGGS)ₙ (SEQ ID NO:34), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGSG (SEQ ID NO:35), GGSGG (SEQ ID NO:36), GSGSG (SEQ ID NO:37), GSGGG (SEQ ID NO:38), GGGSG (SEQ ID NO:39), GSSSG (SEQ ID NO:40), and the like. In some embodiments, a linker employed in the chimeric TCR may be a GGGGSGGGGSGGGGS (SEQ ID NO:41) (G4S) linker. In some embodiments, a linker employed in the chimeric TCR may be a SGSG (SEQ ID NO:42) linker. In some embodiments, a linker employed in the chimeric TCR may be a GSADDAKKDAAKKDGKS (SEQ ID NO:43) linker. In some instances, a GSADDAKKDAAKKDGKS (SEQ ID NO:43) linker may be employed between an antigen binding domain and a domain of a TCR chain in a chain of a chimeric TCR.

The ordinarily skilled artisan will recognize that design of a peptide conjugated to any elements described above can include linkers that are all or partially flexible, such that the linker can include a flexible linker as well as one or more portions that confer less flexible structure.

Antigens and Heterologous Antigen Binding Domains that Bind Thereto

As summarized above, chimeric TCRs of the present disclosure will, in some embodiments, include an attached heterologous antigen binding domain that binds an antigen of interest. By “heterologous” antigen binding domain is meant the antigen binding domain is not naturally present on or within the subject TCR and/or the antigen binding domain binds an antigen which the subject TCR does not naturally bind. Accordingly, subject antigen binding domains may be defined and described in terms of the antigen to which they bind.

As used herein, the term “antigen” will generally refer to one member of a specific binding pair where the molecule that binds the antigen represents the other member of the pair. For example, where a first member of the specific binding pair is fused to a chimeric TCR of the present disclosure, the first member of the specific binding pair binds to a second member of the specific binding pair, where the second member of the specific binding pair is on a different polypeptide from the TCR chain. The second member of the specific binding pair can be present on the surface of a cell (e.g., as an individual polypeptide expressed on the surface of a cell). The second member of the specific binding pair can be presented in the context of a protein complex (e.g., a peptide presented in the context of MHC).The second member of the specific binding pair can be immobilized on an insoluble support. The second member of the specific binding pair can be soluble. The second member of the specific binding pair can be present in an extracellular environment (e.g., extracellular matrix). The second member of the specific binding pair can be present in an artificial matrix. The second member of the specific binding pair can be present in an acellular environment.

Suitable antigen binding domains, discussed in more detail below, may include any appropriate member of a specific binding pair or a fragment thereof that includes the antigen/ligand/receptor binding domain Such members include but are not limited to e.g., members of antigen-antibody binding pairs, members of ligand-receptor binding pairs, scaffold protein pairs and the like. Thus, a member of a specific binding pair suitable for use in a chimeric TCR of the present disclosure includes an antigen, an antibody, a ligand, a ligand-binding receptor and the like.

In some instances, an antigen binding domain employed in a chimeric TCR of the present disclosure may bind a multi-specific antigen. In this way, in some instances, the antigen binding domain of a chimeric TCR may bind an antigen that subsequently binds a second molecule. In some instances, the antigen binding domain employed in a chimeric TCR may itself be multi-specific, binding more than one antigen. In some instances, useful antigen binding domains include those that target two molecules and are thus bi-specific. In some instances, useful antigen binding domains include those that bind an antigen that is bi-specific. Examples of bi-specific molecules, i.e., molecules that bind two different antigens, and the antigens to which they are targeted include but are not limited to e.g., those described in U.S. Pat. No. 9,233,125 and U.S. Patent Application Pub. No. US 20150307564 A1; the disclosures of which are incorporated herein by reference in their entirety.

In some instances, the antigen binding domain of a subject chimeric TCR may bind an “adaptor molecule”. As used herein, the term adaptor molecule will generally refer to a multi-specific molecule that binds the antigen binding domain of a chimeric TCR and has at least one other binding partner. For example, in some instances, an adaptor molecule may be bi-specific, binding the antigen binding domain of the chimeric TCR and one other molecule. In this way, an adaptor to which the antigen binding domain of a chimeric TCR binds may mediate association of the chimeric TCR and the second molecule to which the adaptor binds and/or a cell expressing the second molecule to which the adaptor binds.

Useful adaptor molecules to which the antigen binding domain of a chimeric TCR of the present disclosure may binding will vary and may include but are not limited to e.g., protein dimerizers, chimeric bispecific binding members (e.g., bi-specific T cell engagers (BiTEs)), and the like.

Protein dimerizers generally include polypeptide pairs that dimerize, e.g., in the presence of or when exposed to a dimerizing agent. The dimerizing polypeptide pairs of a protein dimerizer may homo-dimerize or hetero-dimerize (i.e., the dimerizing polypeptide pairs may include two of the same polypeptide that form a homodimer or two different polypeptides that form a heterodimer). Non-limiting pairs of protein dimerizers (with the relevant dimerizing agent in parentheses) include but are not limited to e.g., FK506 binding protein (FKBP) and FKBP (rapamycin); FKBP and calcineurin catalytic subunit A (CnA) (rapamycin); FKBP and cyclophilin (rapamycin); FKBP and FKBP-rapamycin associated protein (FRB) (rapamycin); gyrase B (GyrB) and GyrB (coumermycin); dihydrofolate reductase (DHFR) and DHFR (methotrexate); DmrB and DmrB (AP20187); PYL and ABI (abscisic acid); Cry2 and CIB 1 (blue light); GAI and GID1 (gibberellin); and the like. Further description, including the amino acid sequences, of such protein dimerizers is provided in U.S. Patent Application Publication No. US 2015-0368342 A1; the disclosure of which is incorporated herein by reference in its entirety.

Useful protein dimerizers also include those nuclear hormone receptor derived protein dimerizers that dimerize in the presence of a dimerizing agent described in PCT Patent Application Serial Number US2017/012634; the disclosure of which is incorporated by reference herein in its entirety, and the like. Such nuclear hormone receptor derived dimerizers will generally include a first member of the dimerization pair that is a co-regulator of a nuclear hormone receptor and a second member of the dimerization pair comprises an LBD of the nuclear hormone receptor.

As noted above, in some instances, a chimeric bispecific binding member may find use as an adaptor molecule. As used herein, by “chimeric bispecific binding member” is meant a chimeric polypeptide having dual specificity to two different binding partners (e.g., two different antigens). Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

In some instances, a chimeric bispecific binding member may be a bispecific T cell engager (BiTE). A BiTE is generally made by fusing a specific binding member (e.g., a scFv) that binds an immune cell antigen to a specific binding member (e.g., a scFv) that binds a cancer antigen (e.g., a tumor associated antigen, a tumor specific antigen, etc.). For example, an exemplary BiTE includes an anti-CD3 scFv fused to an anti-tumor associated antigen (e.g., EpCAM, CD19, etc.) scFv via a short peptide linker (e.g., a five amino acid linker, e.g., GGGGS (SEQ ID NO:44)). In some instances, a BiTE suitable for use as herein described includes e.g., an anti-CD3×anti-CD19 BiTE (e.g., Blinatumomab), an anti-EpCAM×anti-CD3 BiTE (e.g., MT110), an anti-CEA×anti-CD3 BiTE (e.g., MT111/MEDI-565), an anti-CD33×anti-CD3 BiTE, an anti-HER2 BiTE, an anti-EGFR BiTE, an anti-IgE BiTE, and the like.

### Antigens

A wide variety of antigens may be targeted with a chimeric TCR of the present disclosure. For example, chimeric TCRs may be redirected to any antigen through the use of an antigen binding domain that binds the antigen. In some instances, antigens of interest include but are not limited to e.g., cancer antigens (including e.g., cancer-specific antigens, cancer-associated antigens, and the like), infectious disease antigens, and the like. Antigens of interest will generally include any antigen to which one may desire to specifically target an immune cell response.

A chimeric TCR of the present disclosure may target a single antigen or multiple antigens, including e.g., one or more antigens, two or more antigens, three or more antigens, four or more antigens, five or more antigens and the like. In some instances, where multiple antigens are targeted a subject chimeric TCR may target e.g., two or more cancer antigens, two or more infectious disease antigens, and the like. In some instances, where a chimeric TCR targets two or more cancer antigens or two or more infectious disease antigens, the two or more antigens may target the same cancer or the same infectious disease, respectively.

Where a chimeric TCR of the present disclosure targets two or more antigens, the antigens targeted may vary. A chimeric TCR targeting two or more antigens may target essentially any combination of antigens, including but not limited to e.g., where the combination of antigens is a combination two or more antigens described herein. In some instances, a chimeric TCR may include two or more different antigen binding domains each directed to a different antigen, where the actual number of domains may range from 2 to 5 or more, including but not limited to e.g., 2, 3, 4, 5, etc. In some instances, a chimeric TCR may include two different antigen binding domains each directed to a different antigen, including e.g., where the two antigens targeted are two antigens described herein. For example, in some instances, a chimeric TCR of the present disclosure may target CD19 and CD20. As another example, in some instances, a chimeric TCR of the present disclosure may target CD19 and CD22.

Antigens will generally be targeted through the use of a heterologous antigen binding domain, discussed in more detail below. Antigen binding domains, as described herein, may be wild-type or may be mutated or synthetic and, accordingly may bind wild-type as well as mutated and synthetic antigen. In some instances, the binding partner/target/antigen bound by an antigen binding domain may be mutated as compared to the wild-type binding partner/target/antigen. In some instances, an antigen binding domain that recognizes a mutated antigen may not specifically bind the wild-type antigen. In some instances, an antigen binding domain that recognizes a mutated antigen may bind the wild-type antigen. In some instances, the mutated antigen binding domain may bind the wild-type antigen with lower affinity as compared to its binding affinity with the mutated antigen.

Any antigen, including e.g., those described herein, may be mutated and the corresponding antigen binding partner may be similarly mutated. Accordingly, a chimeric TCR of the instant disclosure may include an antigen binding domain that specifically binds a mutated (i.e., non-wild-type) binding partner. Non-limiting examples of mutated binding partners include but are not limited to e.g., mutated antigens, mutated cancer antigens, mutated auto-antigens, mutated extracellular antigens, mutated extracellular cancer antigens, mutated extracellular auto-antigens, mutated surface antigens, mutated surface cancer antigens, mutated surface auto-antigens, peptide-MHC complexes presenting a mutated antigen peptide, peptide-MHC complexes presenting a mutated cancer antigen peptide, peptide-MHC complexes presenting a mutated auto-antigen peptide, and the like.

Cancers commonly involve mutated proteins that are associated with the disease. Genes commonly mutated in cancers include e.g., ABI1, ABL1, ABL2, ACKR3, ACSL3, ACSL6, AFF1, AFF3, AFF4, AKAP9, AKT1, AKT2, ALDH2, ALK, AMER1, APC, ARHGAP26, ARHGEF12, ARID1A, ARID2, ARNT, ASPSCR1, ASXL1, ATF1, ATIC, ATM, ATP1A1, ATP2B3, ATRX, AXIN1, BAP1, BCL10, BCL11A, BCL11B, BCL2, BCL3, BCL6, BCL7A, BCL9, BCOR, BCR, BIRC3, BLM, BMPR1A, BRAF, BRCA1, BRCA2, BRD3, BRD4, BRIP1, BTG1, BUB1B, C15orf65, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274, CD74, CD79A, CD79B, CDC73, CDH1, CDH11, CDK12, CDK4, CDK6, CDKN2A, CDKN2C, CDX2, CEBPA, CEP89, CHCHD7, CHEK2, CHIC2, CHN1, CIC, CIITA, CLIP1, CLP1, CLTC, CLTCL1, CNBP, CNOT3, CNTRL, COL1A1, COL2A1, COX6C, CREB1, CREB3L1, CREB3L2, CREBBP, CRLF2, CRTC1, CRTC3, CSF3R, CTNNB1, CUX1, CYLD, DAXX, DCTN1, DDB2, DDIT3, DDX10, DDX5, DDX6, DEK, DICER1, DNM2, DNMT3A, EBF1, ECT2L, EGFR, EIF3E, EIF4A2, ELF4, ELK4, ELL, ELN, EML4, EP300, EPS15, ERBB2, ERC1, ERCC2, ERCC3, ERCC4, ERCC5, ERG, ETV1, ETV4, ETV5, ETV6, EWSR1, EXT1, EXT2, EZH2, EZR, FAM46C, FANCA, FANCC, FANCD2, FANCE, FANCF, FANCG, FAS, FBXO11, FBXW7, FCGR2B, FCRL4, FEV, FGFR1, FGFR1OP, FGFR2, FGFR3, FH, FHIT, FIP1L1, FLCN, FLI1, FLT3, FNBP1, FOXA1, FOXL2, FOXO1, FOXO3, FOXO4, FOXP1, FSTL3, FUBP1, FUS, GAS7, GATA1, GATA2, GATA3, GMPS, GNA11, GNAQ, GNAS, GOLGA5, GOPC, GPC3, GPHN, H3F3A, H3F3B, HERPUD1, HEY1, HIP1, HIST1H4I, HLA-A, HLF, HMGA1, HMGA2, HNF1A, HNRNPA2B1, HOOK3, HOXA11, HOXA13, HOXA9, HOXC11, HOXC13, HOXD11, HOXD13, HRAS, HSP9OAA1, HSP90AB1, IDH1, IDH2, IKZF1, IL2, IL21R, IL6ST, IL7R, IRF4, ITK, JAK1, JAK2, JAK3, JAZF1, JUN, KAT6A, KAT6B, KCNJ5, KDM5A, KDM5C, KDM6A, KDR, KDSR, KIAA1549, KIAA1598, KIF5B, KIT, KLF4, KLF6, KLK2, KMT2A, KMT2C, KMT2D, KRAS, KTN1, LASP1, LCK, LCP1, LHFP, LIFR, LMNA, LMO1, LMO2, LPP, LRIG3, LSM14A, LYL1, MAF, MAFB, MALT1, MAML2, MAP2K1, MAP2K2, MAP2K4, MAX, MDM2, MDM4, MECOM, MED12, MEN1, MET, MITF, MKL1, MLF1, MLH1, MLLT1, MLLT10, MLLT11, MLLT3, MLLT4, MLLT6, MN1, MNX1, MPL, MSH2, MSH6, MSI2, MSN, MTCP1, MUC1, MUTYH, MYB, MYC, MYCL, MYCN, MYD88, MYH11, MYH9, MYO5A, NAB2, NACA, NBN, NCKIPSD, NCOA1, NCOA2, NCOA4, NDRG1, NF1, NF2, NFATC2, NFE2L2, NFIB, NFKB2, NIN, NKX2-1, NONO, NOTCH1, NOTCH2, NPM1, NR4A3, NRAS, NRG1, NSD1, NT5C2, NTRK1, NTRK3, NUMA1, NUP214, NUP98, NUTM1, NUTM2A, NUTM2B, OLIG2, OMD, P2RY8, PAFAH1B2, PALB2, PATZ1, PAX3, PAX5, PAX7, PAX8, PBRM1, PBX1, PCM1, PCSK7, PDCD1LG2, PDE4DIP, PDGFB, PDGFRA, PDGFRB, PER1, PHF6, PHOX2B, PICALM, PIK3CA, PIK3R1, PIM1, PLAG1, PLCG1, PML, PMS1, PMS2, POT1, POU2AF1, POU5F1, PPARG, PPFIBP1, PPP2R1A, PRCC, PRDM1, PRDM16, PRF1, PRKAR1A, PRRX1, PSIP1, PTCH1, PTEN, PTPN11, PTPRB, PTPRC, PTPRK, PWWP2A, RABEP1, RAC1, RAD21, RAD51B, RAFT, RALGDS, RANBP17, RAP1GDS1, RARA, RB1, RBM15, RECQL4, REL, RET, RHOH, RMI2, RNF213, RNF43, ROS1, RPL10, RPL22, RPL5, RPN1, RSPO2, RSPO3, RUNX1, RUNX1T1, SBDS, SDC4, SDHAF2, SDHB, SDHC, SDHD, SEPT5, SEPT6, SEPT9, SET, SETBP1, SETD2, SF3B1, SFPQ, SH2B3, SH3GL1, SLC34A2, SLC45A3, SMAD4, SMARCA4, SMARCB1, SMARCE1, SMO, SOCS1, SOX2, SPECC1, SRGAP3, SRSF2, SRSF3, SS18, SS18L1, SSX1, SSX2, SSX2B, SSX4, SSX4B, STAG2, STAT3, STAT5B, STAT6, STIL, STK11, SUFU, SUZ12, SYK, TAF15, TAL1, TAL2, TBL1XR1, TCEA1, TCF12, TCF3, TCF7L2, TCL1A, TERT, TET1, TET2, TFE3, TFEB, TFG, TFPT, TFRC, THRAP3, TLX1, TLX3, TMPRSS2, TNFAIP3, TNFRSF14, TNFRSF17, TOP1, TP53, TPM3, TPM4, TPR, TRAF7, TRIM24, TRIM27, TRIM33, TRIP11, TRRAP, TSC1, TSC2, TSHR, TTL, U2AF1, UBR5, USP6, VHL, VTI1A, WAS, WHSC1, WHSC1L1, WIF1, WRN, WT1, WWTR1, XPA, XPC, XPO1, YWHAE, ZBTB16, ZCCHC8, ZMYM2, ZNF331, ZNF384, ZNF521 and ZRSR2. In some instances, an antigen binding domain binds to the mutated version of a gene that is commonly mutated in cancer, including but not limited to e.g., those listed above. In some instances, an antigen binding domain binds to a peptide-MHC complex presenting a mutated cancer antigen peptide derived from the mutated version of a gene that is commonly mutated in cancer, including but not limited to e.g., those listed above. In some instances, an antigen binding domain binds to a peptide-MHC complex presenting a mutant KRAS peptide.

In some instances, a binding partner/specific binding member pair may be orthogonalized. As used herein, by “orthogonalized” is meant modified from their original or wild-type form such that the orthogonal pair specifically bind one another but do not specifically or substantially bind the non-modified or wild-type components of the pair. Any binding partner/specific binding pair may be orthogonalized, including but not limited to e.g., those binding partner/specific binding pairs described herein.

In some instances, an antigen targeted by a chimeric TCR may be an antigen expressed on the surface of a target cell. For example, in some instances, a chimeric TCR may target a cell surface antigen expressed by a target cell. Such surface antigens will vary and will generally include those antigens expressed on the surface of a cell that are not complexed with major histocompatibility complex (MHC), as described below. In some instances, a chimeric TCR may target a cell surface antigen associated with cancer, which may be referred to herein as a cell surface cancer antigen.

An antigen-binding domain suitable for use in a chimeric TCR of the present disclosure can have a variety of antigen-binding specificities. In some cases, the antigen-binding domain is specific for an epitope present in an antigen that is expressed by (synthesized by) a cancer cell, i.e., a cancer cell associated antigen. Antigens bound by an antigen-binding domain may or may not be presented in the context of MHC, e.g., antigens may be present outside the context of MHC such as in the case of a cell surface antigen or may be presented in the context of MHC such as in the case of a peptide-MHC. The cancer cell associated antigen can be an antigen associated with, e.g., a breast cancer cell, a B cell cancer, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, a myeloma (i.e., a plasma cell cancer), etc. A cancer specific antigen is generally not expressed by non-cancerous cells. In some instances, a cancer specific antigen may be minimally expressed by one or more non-cancerous cell types. By “minimally expressed” is meant that the level of expression, in terms of either the per-cell expression level or the number of cells expressing, minimally, insignificantly or undetectably results in binding of the specific binding member to non-cancerous cells expressing the antigen.

Non-limiting examples of antigens to which an antigen-binding domain of a subject chimeric TCR can bind include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

In some instances, an antigen to which an antigen-binding domain of a subject chimeric TCR is directed may be an antigen selected from: AFP, BCMA, CD10, CD117, CD123, CD133, CD138 , CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, Glypican 3, GPC3, HER-2, kappa immunoglobulin, LeY, LMP1, mesothelin, MG7, MUC1, NKG2D-ligands, PD-L1, PSCA, PSMA, ROR1, ROR1R, TACI and VEGFR2 and may include, e.g., an antigen binding-domain of or derived from a CAR currently or previously under investigation in one or more clinical trials.

In some instances, the antigen binding domain of a chimeric TCR of the instant disclosure may target a cancer-associated antigen. In some instances, the antigen binding domain of a chimeric TCR of the instant disclosure may include an antibody specific for a cancer associated antigen. Non-limiting examples of cancer associated antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some instances, a specific binding member of a chimeric TCR may specifically bind a target comprising a fragment of a protein (e.g., a peptide) in conjunction with a major histocompatibility complex (MHC) molecule. As MHC molecules present peptide fragments of both intracellularly expressed and extracellularly expressed proteins, specific binding members directed to MHC-peptide complexes allows for the targeting of intracellular antigens as well as extracellularly expressed antigens.

Intracellularly expressed target proteins (e.g., cytoplasmically expressed (i.e., cytoplasmic proteins), nuclearly expressed (i.e., nuclear proteins), etc.) may be referred to as intracellular antigens (e.g., cytoplasmic antigens, nuclear antigens, etc.). Accordingly, specific binding members of the subject disclosure may be specific for intracellular antigen fragments complexed with MHC, e.g., a peptide-MHC complex, also, in some instances, described as a human leukocyte antigen (HLA)-peptide complex.

All endogenous cellular proteins (host or pathogen) are processed into short peptides for display at the cell surface in association with HLA molecules. Peptide-HLA class I complexes displayed on the cell surface play an important role in the T-cell mediated immune response. The approximately 9-residue long peptides originate from proteins that are digested by the proteasome inside the cell. Depending on whether the T-cell receptor recognizes a peptide as self or non-self, an immune response may be initiated. Peptide-HLA complexes displayed specifically on the surface of cancer cells provide an excellent opportunity to develop targeted cancer therapeutics, including engineered T-cells or “TCR-like” antibodies. The advent of various technologies, including e.g., MHC based tetramer technology, have advanced the ability to develop TCR-like anti-HLA/peptide specific antibodies.

In some instances, the binding partner of an antigen binding domain of the subject chimeric TCRs may include peptide-MHC or HLA/peptide complexes. In some instances, the antigen binding domain of the subject chimeric TCR is specific for a MHC class I MHC-peptide complex including e.g., a HLA-A/peptide complex, a HLA-B/peptide complex or a HLA-C/peptide complex. In some instances, the antigen binding domain of the subject chimeric TCR is specific for a MHC class II MHC-peptide complex including e.g., a HLA-DPA1/peptide complex, a HLA-DPB1/peptide complex, a HLA-DQA1/peptide complex, a HLA-DQB1/peptide complex, a HLA-DRA/peptide complex or a HLA-DRB1/peptide complex. In some instances, the antigen binding domain of the subject chimeric TCR is specific for a MHC class III MHC-peptide complex.

Peptide-MHC Binding partners will generally include a target protein fragment peptide presented in the context of MHC. Such peptides vary in size depending on numerous factors including e.g., the class of MHC molecule to which they are bound. For example, class I MHC associated peptides are generally 9 aa in length but may vary in size including less than about 9 aa or more than about 9 aa including but not limited to e.g., 8 aa or 10 aa. Whereas, class II MHC associated peptides may also vary in size from about 13 aa to about 25 aa, including but not limited to e.g., 13 aa, 14 aa, 15 aa, 16 aa, 17 aa, 18 aa, 19 aa, 20 aa, 21 aa, 22 aa, 23 aa, 24 aa or 25 aa.

Exemplary protein targets to which a antigen binding domain targeting a peptide-MHC complex may be directed as well as exemplary peptides in the context of MHC for each protein target are provided in Table 2 below.

TABLE 2
anti-peptide-MHC targets:

| Target | Exemplary Peptides | HLA | References |
|---|---|---|---|
| WT1 | RMFPNAPYL (SEQ ID NO: 45) | HLA-A2 | Leukemia. (2015) 29(11): 2238-47 |
| KRAS and | KLVVVGAGGV (SEQ ID NO: 46); | HLA-A2; | Proc Natl Acad Sci USA. (2015) 112(32) |
| KRAS mutants | KLVVVGAVGV (SEQ ID NO: 47); | HLA-A3 | |
| (e.g., G12V & | KLVVVGACGV (SEQ ID NO:48); | | |
| G12C) | KLVVVGADGV (SEQ ID NO: 49); | | |
| | VVGAVGVGK (SEQ ID NO: 50); | | |
| | VVGACGVGK (SEQ ID NO: 51); | | |
| | VVGAGGVGK (SEQ ID NO: 52) | | |
| EGFP and EGFP | KITDFGLAK (SEQ ID NO: 53); | HLA-A3 | Proc Natl Acad Sci USA. (2015) 112(32) |
| mutants (e.g., | KITDFGRAK (SEQ ID NO: 54); | | |
| L858R) | | | |
| PR1 | VLQELNVTV (SEQ ID NO: 55) | HLA-A2 | Cytotherapy. (2016) 18(8): 985-94 |
| MAGE-A1 | EADPTGHSY (SEQ ID NO: 56) | HLA-A1 | Blood. (2011) 117(16): 4262-4272 |
| P53 | LLGRNSFEV (SEQ ID NO: 57); | HLA-A2 | Gene Ther. (2001) 8(21): 1601-8 |
| | STTPPPGTRV (SEQ ID NO: 58) | | |
| MART-1 | ELAGIGILTV (SEQ ID NO: 59) | HLA-A2 | Biomark Med. (2010) 4(4): 496-7 |
| gp100 | IMDQVPFSV (SEQ ID NO: 60) | HLA-A2 | Biomark Med. (2010) 4(4): 496-7 |
| CMVpp65 | NLVPMVATV (SEQ ID NO: 61) | HLA-A2 | Biomark Med. (2010) 4(4): 496-7 |
| HIVVpr | AIIRILQQL (SEQ ID NO: 62) | HLA-A2 | Biomark Med. (2010) 4(4): 496-7 |
| HA-1H | VLHDDLLEA (SEQ ID NO: 63); | HLA-A2 | Biomark Med. (2010) 4(4): 496-7 |
| | VLRDDLLEA (SEQ ID NO: 64) | | |
| NY-ESO-1 | SLLMWITQV (SEQ ID NO: 65) | HLA-A2 | Gene Ther. (2014) 21(6): 575-84 |
| EBNA3C | LLDFVRFMGV (SEQ ID NO: 66) | HLA-A2 | Proc Natl Acad Sci USA. (2009) |
| | | | 106(14): 5784-8 |
| AFP | FMNKFIYEI (SEQ ID NO: 67) | HLA-A2 | Cancer Gene Ther. (2012) 19(2): 84-100 |
| Her2 | KIFGSLAFL (SEQ ID NO: 68) | HLA-A2 | Clin Cancer Res. (2016) pii: clincanres |
| | | | 1203.2016 |
| hCG-beta | GVLPALPQV (SEQ ID NO: 69) | HLA-A2 | J Natl Cancer Inst. (2013) 105(3): 202-18 |
| HBV Env183-91 | FLLTRILTI (SEQ ID NO: 70) | HLA-A2 | J Immunol. (2006) 177(6): 4187-95 |

In some instances, the antigen binding domain of a chimeric TCR of the instant disclosure specifically binds a peptide-MHC having an intracellular cancer antigen peptide of Table 2. In some instances, the antigen binding domain of a chimeric TCR of the instant disclosure specifically binds a WT1 peptide-MHC. In some instances, the antigen binding domain of a chimeric TCR of the instant disclosure specifically binds a NY-ESO-1 peptide-MHC.

Specific antigens, and the amino acid sequences thereof, that may find use in the subject chimeric TCRs include those that have been previously described and utilized in various contexts including but not limited to the contexts of antibodies, engineered TCRs and CARs, including e.g., those described in those described in U.S. Patent Application Publication No. US 2015-0368342 A1; U.S. Patent Application No. 62/378,614; PCT Publication No. WO 2014/127261 and PCT Application Nos. US2016/049745; US2016/062612; the disclosures of which are incorporated herein by reference in their entirety.

### Antigen Binding Domains

The antigen binding domain of a chimeric TCR, where present, will be an extracellular component of the chimeric TCR. In some instances, only one chain of a TCR alpha and beta chain pair will include an antigen binding domain, e.g., only the TCR alpha chain includes an antigen binding domain or only the TCR beta chain includes an antigen binding domain In some instances, both the TCR alpha and beta chains of a TCR alpha and beta chain pair will each include an antigen binding domain As noted above, antigen binding domains useful in the subject chimeric TCRs include those that serve as one member of a specific binding pair. Accordingly, antigen binding domains that may be employed include but are not limited to e.g., ligand, receptor, antigen and antibody polypeptides or polypeptide fragments that include the antigen/ligand/receptor binding portions thereof. Antigen binding domains may be or may be derived from antigen-antibody binding pairs, ligand-receptor binding pairs, and the like.

Suitable antigen binding domains of ligand-receptor binding pairs can be any ligand binding domain of a receptor or receptor binding domain of a ligand, a wide variety of which are known in the art. In some cases, a member of a specific binding pair suitable for use in a subject chimeric TCR is a ligand for a receptor. Ligands include, but are not limited to, cytokines (e.g., IL-13, etc.); growth factors (e.g., heregulin; vascular endothelial growth factor (VEGF); and the like); an integrin-binding peptide (e.g., a peptide comprising the sequence Arg-Gly-Asp); and the like.

Where the member of a specific binding pair in a subject chimeric TCR is a ligand, the chimeric

TCR can be activated in the presence of a receptor for the ligand. For example, where the ligand is VEGF, the second member of the specific binding pair can be a VEGF receptor, including a soluble VEGF receptor. As another example, where the ligand is heregulin, the second member of the specific binding pair can be Her2.

In some cases, a member of a specific binding pair suitable for use in a subject chimeric TCR is a receptor, or domain thereof or a co-receptor, for a ligand. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); Her2; CD27; a natural cytotoxicity receptor (NCR) (e.g., NKP30 (NCR3/CD337) polypeptide (receptor for HLA-B-associated transcript 3 (BAT3) and B7-H6); etc.); etc.

Suitable antigen binding domains of antigen-antibody binding pairs can be any antigen-binding polypeptide of antigen-antibody binding pair origin, a wide variety of which are known in the art. In some instances, the antigen-binding domain is a single chain Fv (scFv). Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use.

One non-limiting example of a scFv antigen binding domain is an anti-mesothelin scFv. In some instances, an anti-mesothelin scFv has the following amino acid sequence or an amino acid sequence having at least 85% sequence identity (including at least 90%, at least 95% or at least 99%) with the following amino acid sequence: SGPELEKPGASVKISCKASGYSFTGYTMNWVKQSHGKSLEWIGLITPYNGASSYNQKFRGKATL TVDKSSSTAYMDLLSLTSEDSAVYFCARGGYDGRGFDYWGQGTTVTVSSGGGGSGGGGSSGG GSDIELTQSPAIMSASPGEKVTMTCSASSSVSYMHWYQQKSGTSPKRWIYDTSKLASGVPGRFS GSGSGNSYSLTISSVEAEDDATYYCQQWSKHPLTYGAGTKLEIKAS (SEQ ID NO:71), where the subject scFv is composed of two variable regions (i.e., a first variable domain and a second variable domain) separated by the (G₄5)₃ linker sequence GGGGSGGGGSGGGGS (SEQ ID NO:41). An ordinary skilled artisan will readily appreciate that various other anti-mesothelin scFv may be derived from the above sequence including e.g., where the linker is modified or a different linker is used to link the first and second variable regions.

In some instances, the antigen binding domain of a chimeric TCR may include only one specific binding member and may be specific for only one antigen. In some instances, the antigen binding domain of a chimeric TCR may by mono-specific.

In some instances, the antigen binding domain of a chimeric TCR may by multi-specific, including e.g., bispecific. In some instances, a bispecific antigen binding domain of a chimeric TCR may include a bispecific chimeric binding member, or portion thereof, including e.g., those described herein, including but not limited to e.g., a bispecific antibody. In some instances, a bispecific antigen binding domain may include two specific binding domains that are linked, including e.g., directly linked to each other or linked via a linker.

In some instances, the antigen binding domain of a chimeric TCR may include more than one specific binding member, including two or more specific binding members where the two or more specific binding members may be linked (either directly or indirectly, e.g., through the use of a linker) to each other or they may each be linked (either directly or indirectly, e.g., through the use of a linker) to another component of the chimeric TCR.

Multi-specific antigen binding domains may recognize or bind to any combination of binding partners and thus may target any combination of targets, including but not limited to e.g., those antigens and targets described herein. Accordingly, e.g., a bispecific antigen binding domain may target two different antigens including but not limited to e.g., two different intracellular antigens, two different extracellular (e.g., surface expressed) antigens or an intracellular antigen and an extracellular (e.g., surface expressed) antigen. In some instances, a bispecific antigen binding domain may include two specific binding members, including e.g., two specific binding members described herein, that each bind an antigen, including e.g., an antigen described herein.

The specific binding domains of a multi-specific antigen binding domain may each activate the chimeric TCR of which they are a part. The specific binding domains of a bispecific antigen binding domain may each activate the chimeric polypeptide of which they are a part. In some instances, multi-specific or bispecific binding domains may find use as part of a molecular circuit as described herein including e.g., as an OR-gate of a circuit described herein.

Specific antigen binding domains, and the amino acid sequences thereof, that may find use in the subject chimeric TCRs include those that have been previously described and utilized in various contexts including but not limited to the contexts of antibodies, engineered TCRs and CARs, including e.g., those described in U.S. Patent Application Publication No. US 2015-0368342 A1; U.S. Patent Application No. 62/378,614; PCT Publication No. WO 2014/127261 and PCT Application Nos. US2016/049745; US2016/062612; the disclosures of which are incorporated herein by reference in their entirety.

### Nucleic Acids and Expression Vectors

As summarized above, the present disclosure also provides nucleic acids and expression vectors. Nucleic acids of the present disclosure include those encoding one or more modified TCR chains as well as nucleic acids encoding a chimeric TCR. Recombinant expression vectors of the present disclosure include those comprising one or more of the described nucleic acids. A nucleic acid comprising a nucleotide sequence encoding a chimeric TCR of the present disclosure will in some embodiments be DNA, including, e.g., a recombinant expression vector. A nucleic acid comprising a nucleotide sequence encoding a chimeric TCR of the present disclosure will in some embodiments be RNA, e.g., in vitro synthesized RNA.

In some cases, a nucleic acid of the present disclosure comprises a nucleotide sequence encoding only an alpha chain of a chimeric TCR, e.g., a modified alpha of a chimeric TCR of the present disclosure. In some cases, a nucleic acid of the present disclosure comprises a nucleotide sequence encoding only a beta chain of a chimeric TCR, e.g., a modified beta chain of a chimeric TCR of the present disclosure. In some cases, a nucleic acid of the present disclosure comprises a nucleotide sequence encoding all or both parts of a chimeric TCR of the present disclosure, including e.g., both a modified alpha chain and a modified beta chain. Nucleic acid sequences of the subject nucleic acids may be operably linked to transcriptional control elements such as promoters, enhancers, etc.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements, may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

Furin cleavage sites will vary, where the minimal cleavage site is Arg-X-X-Arg (SEQ ID NO:86). However, the enzyme prefers the site Arg-X-(Lys/Arg)-Arg (SEQ ID NO:87). An additional arginine at the P6 position appears to enhance cleavage (Arg-X-X-Arg-X-Arg (SEQ ID NO:88) or Arg-X-(Lys/Arg)-Arg-X-Arg (SEQ ID NO:89)). Furin, and thus furin cleavage, is inhibited by certain reaction compounds including e.g., EGTA, al-Antitrypsin Portland and polyarginine compounds. In some instances, a furin cleavage site encoded by a bicistronic-facilitating sequence may be RKRR (SEQ ID NO:72).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence EGRGSLLTCGDVEENPGP (SEQ ID NO:73).

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag such as e.g., GKPIPNPLLGLDST (SEQ ID NO:74).

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21):1411-1423; Martin et al. (2006) BMC Biotechnology. 6:4; the disclosures of which are incorporated herein by reference in their entirety.

Nucleic acids and/or expression vectors encoding a chimeric TCR of the present disclosure may include sequence encoding one or more epsilon, delta, gamma, and/or zeta chains, or in some instances, a nucleic acid encoding a chimeric TCR may not include sequence encoding one or more epsilon, delta, gamma and/or zeta chains and may instead rely upon endogenously expressed epsilon, delta, gamma and/or zeta chains.

Nucleic acids encoding chimeric TCRs, and thus the expressed chimeric TCRs themselves, may include one or more additional nucleic acid sequences encoding one or more additional polypeptides, which may be referred to as additional polypeptide domains.

Suitable additional polypeptide domains that may be encoded by the subject nucleic acids include but are not limited to e.g., those sequences encoding signal sequences, epitope tags, affinity domains, detectable signal-producing polypeptides, and the like. Signal sequences that are suitable for use in a subject chimeric TCR include any eukaryotic signal sequence, including a naturally-occurring signal sequence, a synthetic (e.g., man-made) signal sequence, etc. In some instances, a signal sequence employed may be or may be derived from the following signal sequence amino acid sequence:

(SEQ ID NO: 75)
MALPVTALLLPLALLLHAARP.

Suitable epitope tags include, but are not limited to, hemagglutinin (HA; e.g., YPYDVPDYA (SEQ ID NO:81); FLAG (e.g., DYKDDDDK (SEQ ID NO:79); c-myc (e.g., EQKLISEEDL; SEQ ID NO:78), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO:76), HisX6 (HHHHHH) (SEQ ID NO:77), C-myc (EQKLISEEDL) (SEQ ID NO:78), Flag (DYKDDDDK) (SEQ ID NO:79), StrepTag (WSHPQFEK) (SEQ ID NO:80), hemagglutinin, e.g., HA Tag (YPYDVPDYA) (SEQ ID NO:81), GST, thioredoxin, cellulose binding domain, RYIRS (SEQ ID NO:82), Phe-His-His-Thr (SEQ ID NO:83), chitin binding domain, S-peptide, T7 peptide, SH2 domain, C-end RNA tag, WEAAAREACCRECCARA (SEQ ID NO:84), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, 5100 proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Promoters

In some instances, nucleic acids of the present disclosure encoding all or part (e.g., one chain) of a subject chimeric TCR may include one or more coding sequences operably linked to one or more promoters, including e.g., where one or more of the promoters is an inducible promoter. In some instances, a single promoter may be operably linked to a single coding sequence, including where the coding sequence encodes a mono- or a multicistronic (e.g., bicistronic) polypeptide. In some instances, two promoters may be individually operably linked to two different coding sequences, including where the two promoters are the same or different. In some instances, promoters utilized in the subject nucleic acids may be inducible, repressible and/or conditional. In some instances, one or more of the promoters utilized may be cell type specific, including e.g., where one or more of the promoters utilized are immune cell specific promoters.

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to; cytomegalovirus immediate early promoter; herpes simplex virus thymidine kinase promoter; early and late SV40 promoters; promoter present in long terminal repeats from a retrovirus; mouse metallothionein-I promoter; and various art-known promoters.

Suitable promoters for use in prokaryotic host cells include, but are not limited to, a bacteriophage

T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and P_{Lambda} Non-limiting examples of operators for use in bacterial host cells include a lactose promoter operator (Lad repressor protein changes conformation when contacted with lactose, thereby preventing the LacI repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, nucleic acids of the present disclosure include immune cell specific promoters that drive expression in one or more immune cell types, including but not limited to lymphocytes, hematopoietic stem cells and/or progeny thereof (i.e., immune cell progenitors), etc. Any convenient and appropriate promoter of an immune cell specific gene may find use in nucleic acids of the present disclosure. In some instances, an immune cell specific promoter of a nucleic acid of the present disclosure may be a T cell specific promoter. In some instances, an immune cell specific promoter of a nucleic acid of the present disclosure may be a light and/or heavy chain immunoglobulin gene promoter and may or may not include one or more related enhancer elements.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some instances, an immune cell specific promoter of a nucleic acid of the present disclosure may be a viral promoter active in immune cells. As such, in some instances, viral promoters useful in nucleic acids of the present disclosure include viral promoters derived from immune cells viruses, including but not limited to, e.g., lentivirus promoters (e.g., HIV, SIV, FIV, EIAV, or Visna promoters) including e.g., LTR promoter, etc., Retroviridae promoters including, e.g., HTLV-I promoter, HTLV-II promoter, etc., and the like.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

### Conditional Nucleic Acid Components, Constructs and Use Thereof

In some instances, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.), the disclosures of which are incorporated herein by reference.

A nucleotide sequence encoding a subject chimeric TCR can be present in an expression vector and/or a cloning vector. Where a subject chimeric TCR is split between two or more separate polypeptides (e.g., separate alpha and beta chains), nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a chimeric TCR or a chain thereof of the present disclosure will in some embodiments be DNA or RNA, e.g., in vitro synthesized DNA or in vitro synthesized RNA. Methods for in vitro synthesis of DNA/RNA are known in the art; any known method can be used to synthesize DNA/RNA comprising a nucleotide sequence encoding the chimeric TCR or a first and/or a second polypeptide of a chimeric TCR of the present disclosure. Methods for introducing DNA/RNA into a host cell are known in the art. Introducing DNA/RNA comprising a nucleotide sequence encoding a chimeric TCR or a first and/or second polypeptide of a chimeric TCR of the present disclosure into a host cell can be carried out in vitro or ex vivo or in vivo. For example, a host cell (e.g., an NK cell, a cytotoxic T lymphocyte, etc.) can be transduced, transfected or electroporated in vitro or ex vivo with DNA/RNA comprising a nucleotide sequence encoding the chimeric TCR or a first and/or second polypeptide of a chimeric TCR of the present disclosure.

### Immune Cells

As summarized above, the present disclosure also provides immune cells Immune cells of the present disclosure include those that contain one or more of the described nucleic acids, expression vectors, modified TCR chains and/or chimeric TCRs Immune cells of the present disclosure include mammalian immune cells including e.g., those that are genetically modified to produce a chimeric TCR of the present disclosure or to which a nucleic acid, as described above, has been otherwise introduced. In some instances, the subject immune cells have been transduced with one or more nucleic acids and/or expression vectors to express one or more modified TCR chains or a chimeric TCR of the present disclosure.

Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a T lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts Immune cells also include B cell progenitors such as Pro B cells and B cell lineage derivatives such as plasma cells.

Immune cells expressing a chimeric TCR of the present disclosure may be generated by any convenient method. Nucleic acids encoding one or more chains of a chimeric TCR may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a T lymphocyte, a stem cell, or an NK cell is obtained from an individual; and the cell obtained from the individual is modified to express a chimeric TCR of the present disclosure. The modified cell can thus be redirected to one or more antigens of choice, as defined by the one or more antigen binding domains present on the introduced chimeric TCR. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

Immune cells of the present disclosure, expressing a chimeric TCR having an antigen binding domain that binds an antigen, may become activated upon binding of the antigen to the chimeric TCR. Immune cell activation, as a result of an expressed chimeric TCR binding an antigen, may be measured in a variety of ways, including but not limited to e.g., measuring the expression level of one or more markers of immune cell activation. Useful markers of immune cell activation include but are not limited to e.g., CD25, CD38, CD4OL (CD154), CD69, CD71, CD95, HLA-DR, CD137 and the like. For example, in some instances, upon antigen binding an immune cell expressing a chimeric TCR may become activated and may express a marker of immune cell activation (e.g., CD69) at an elevated level (e.g., a level higher than a corresponding cell not expressing the chimeric TCR). Levels of elevated expression of activated immune cells of the present disclosure will vary and may include a 1-fold or greater increase in marker expression as compared to un-activated control, including but not limited to e.g., a 1-fold increase, a 2-fold increase, a 3-fold increase, a 4-fold increase, etc.

In some instances, a chimeric TCR expressing immune cell, when bound to an antigen, may have increased cytotoxic activity, e.g., as compared to an un-activated control cell that does not express the chimeric TCR. In some instances, activated immune cells expressing a chimeric TCR show 10% or greater cell killing of antigen expressing target cells as compared to un-activated control cells. In some instances, the level of elevated cell killing of activated chimeric TCR expressing immune cells will vary and may range from 10% or greater, including but not limited to e.g., 20% or greater, 30% or greater, 40% or greater, 50% or greater, 60% or greater, 70% or greater, 80% or greater, 90% or greater, etc., as compared to an appropriate control.

### Systems for Expression

The present disclosure includes systems for the expression of the herein described chimeric TCRs. In some instances, expression of one or more chains of a chimeric TCR may be dependent upon one or more inputs, e.g., antigen inputs, as part of a molecular circuit. For example, in some instances, such a system may depend on the presence/binding of a first antigen to trigger the expression of one or more chains of a chimeric TCR. Signaling through the first antigen may be achieved through the use of any binding-triggered transcriptional switch provided binding of the antigen to the binding-triggered transcriptional switch results in transcription of the one or more chains of the chimeric TCR.

Systems involving binding-triggered transcriptional switches, and components thereof, have been described in PCT Publication No. WO 2016/138034, US Patent Application Pub. No. US 2016-0264665 A1 and issued U.S. Pat. Nos. 9,670,281 and 9,834,608; the disclosures of which are incorporated by reference herein in their entirety.

For example, in some instances, a synthetic Notch receptor (i.e., “synNotch”) may be employed as a binding-triggered transcriptional switch that, when bound to its antigen, activates a promoter to which a nucleic acid sequence encoding one or more chains of a chimeric TCR is operably linked. Accordingly, as a non-limiting example, such systems may require the presence of a first antigen (e.g., to which the synNotch binds) for the immune cell to be responsive to one or more second antigens (e.g., to which a chimeric TCR binds). The requirement of particular antigen combinations to generate certain signaling outputs in molecular circuits results in a logic gate.

In some instances, the independent activities and/or induced expression of two or more polypeptides or domains of a single polypeptide may generate a logic gated circuit. Such logic gated circuits may include but are not limited to e.g., “AND gates”, “OR gates”, “NOT gates” and combinations thereof including e.g., higher order gates including e.g., higher order AND gates, higher order OR gates, higher order NOT gates, higher order combined gates (i.e., gates using some combination of AND, OR and/or NOT gates).

“AND” gates include where two or more inputs are required for propagation of a signal. For example, in some instances, an AND gate allows signaling through a first input of a first polypeptide or a first polypeptide domain and a second input dependent upon the output of the first input. In an AND gate two inputs, e.g., two antigens, are required for signaling through the circuit.

“OR” gates include where either of two or more inputs may allow for the propagation of a signal. For example, in some instances, an OR gate allows signaling through binding of either of two different antigens. In an OR gate any one input, e.g., either of two antigens, may induce the signaling output of the circuit. In one embodiment, an OR gate may be achieved through the use of two separate molecules or constructs. In another embodiment, an OR gate may be achieved through the use of a single construct that recognizes two antigens, including e.g., a chimeric TCR having two different antigen binding domains that each bind a different antigen and each binding even can independently activate the chimeric TCR.

“NOT” gates include where an input is capable of preventing the propagation of a signal. For example, in some instances, a NOT gate inhibits signaling through a chimeric TCR of the instant disclosure. In one embodiment, a NOT gate may prevent the expression of a chimeric TCR or a particular chain of a chimeric TCR, e.g., a chain of a chimeric TCR having an antigen binding domain.

In some embodiments, a binding-triggered transcriptional switch (e.g., a synNotch) may be employed to result in an AND gate that incorporates a chimeric TCR of the present disclosure. For example, a nucleic acid sequence encoding a chimeric TCR may be operably linked to a promoter that is responsive to the intracellular domain of the binding-triggered transcriptional switch. Upon binding the first antigen by the binding-triggered transcriptional switch the promoter becomes activated and the expressing cell is then responsive to the antigen to which chimeric TCR binds. Accordingly, immune cell activation through the chimeric TCR requires the first antigen AND the second antigen.

In some embodiments, the individual chains of a chimeric TCR may be split components of a logic gate. For example, in some instances, a first chain of a chimeric TCR may be operably linked to a first promoter responsive to the intracellular domain of a first synNotch (or other binding-triggered transcriptional switch), such that binding of the first antigen to the first synNotch is required for expression of the first chain of the chimeric TCR. Within the same system, a second chain of the chimeric TCR may be operably linked to a second promoter responsive to the intracellular domain of a second synNotch, such that binding of the second antigen to the second synNotch is required for expression of the second chain of the chimeric TCR. Accordingly, assembly of the chimeric TCR requires the first antigen AND the second antigen. Further, immune cell activation through the chimeric TCR of such a system may further require a third antigen to which the chimeric TCR binds. The relevant ordinary skilled will readily understand how such systems may be employed to increase specificity of immune cell activation as well as how the complexity of such systems may be expanded or simplified as desired.

In some instances, multiple antigen binding domains present on a chimeric TCR of the present disclosure may include an OR gate capability to the herein described molecule circuits. For example, in some instances, a chimeric TCR having two different antigen binding domains may be responsive to a first antigen OR a second antigen.

In some instances, such OR gates may be combined with other gates, including an AND gate. For example, a nucleic acid encoding an OR-gate chimeric TCR having two different antigen binding domains may be operably linked to a promoter that is responsive to the intracellular domain of a synNotch which is responsive to a first antigen. As such, upon binding the first antigen, the synNotch drives expression of the chimeric TCR which is responsive to two different antigens, resulting in an AND-OR gate.

Such logic gate circuits may be employed in various combinations to generate any desired result which may take advantage of the particular distribution of employed antigens (e.g., within a subject to be treated). For example, a broadly expressed antigen (e.g., a tissue level antigen) may be employed to trigger expression of a chimeric TCR that is responsive only to a specific cancer antigen. This approach allows for expression of the chimeric TCR only in specific tissues, e.g., a tissue where a cancer is known to be present, and the specificity of the chimeric TCR antigen assures toxicity within the tissue is primarily directed cancer cells. Such an approach may prevent off-target effects, e.g., where cells of a non-target tissue express the “cancer-antigen” but do not express the tissue-level antigen.

Depending on the particular distribution of targeted antigens, such molecular circuits may be designed for desired targeting of target cells while reducing the occurrence of undesirable outcomes such as off-target effects and/or overall unacceptably high levels of cytotoxicity or uncontrolled and widespread immune activation. Accordingly, numerous alternative molecular circuits may be designed and implemented as desired.

### Methods

As summarized above, the present disclosure also provides methods, including methods of using one or more modified TCR chains, one or more chimeric TCRs, one or more nucleic acids encoding one or more modified TCR chains or a chimeric TCR, one or more expression vectors that includes one or more of such nucleic acids and/or one or more of the described immune cells.

In some instances, methods of the present disclosure include methods of killing a target cell. As described above, target cells include those cells that express one or more antigens to which a chimeric TCR of the present disclosure is directed. Accordingly, methods that involve the killing of target cells may include contacting a target cell expressing an antigen with an immune cell expressing a chimeric TCR having an antigen binding domain that binds the antigen. Upon binding the antigen the immune cell may become activated and cytotoxic towards the target cell, resulting in death of the target cell.

A target cell will generally include any cell expressing one or more antigens to which a chimeric TCR is directed. Methods of killing a subject target cell may include contacting the target cell with a chimeric TCR expressing immune cell in various contexts, including e.g., where the target cell is present in vitro, ex vivo or in vivo. For example, in some instances, a target cell may be present in an in vitro culture and chimeric TCR expressing immune cells may be added to the culture to result in killing of the target cell. In some instances, a target cell may be present in an in vivo in a subject and chimeric TCR expressing immune cells may be administered to the subject to result in killing of the target cell within the subject.

In some instances, methods of the present disclosure may include contacting an immune cell with one or more nucleic acids encoding one or more chains of a chimeric TCR as described herein to result in expression of the chimeric TCR by the contacted immune cell. In some instances, a subject method may include contacting an immune cell with one or more nucleic acids resulting in expression of paired chains of a chimeric TCR, where such paired chains may include correspondingly modified (e.g., correspondingly truncated, correspondingly cysteine modified, correspondingly domain swapped, etc.) alpha and beta chains of a chimeric TCR. As described above, such contacting may include the use of a nucleic acid vector, including e.g., a recombinant expression vector or the like.

In some instances, expression of paired chains of a chimeric TCR may result in increased cell surface expression of the chimeric TCR relative to unpaired chains or a TCR containing unpaired chains. In some instances, expression of paired chains of a chimeric TCR may result in increased effectiveness (e.g., increased immune cell activation, increased target cell killing, etc.) of the chimeric TCR relative to unpaired chains or a TCR containing unpaired chains.

As noted above, in some instances, the subject methods may result increased activation of immune cells of the present disclosure, expressing a chimeric TCR having an antigen binding domain that binds an antigen, as compared to control cells not expressing the chimeric TCR. Such increased activation will generally be antigen-specific such that immune cells expressing a chimeric TCR will be specifically activated in the presence of the antigen to which the chimeric TCR binds. Increased activation of the subject immune cells in the present methods may manifest in various ways including where the activation results in the increased expression of one or more immune cell activation markers, including but not limited to e.g., upregulation of one or more of CD25, CD38, CD4OL (CD154), CD69, CD71, CD95, HLA-DR, CD137 and the like. The subject methods may result in increased levels of activated immune cell marker expression of 1-fold or greater (as compared to un-activated control), including but not limited to e.g., 1-fold greater, 2-fold greater, 3-fold greater, 4-fold greater, etc.

In some instances, methods of the present disclosure result in increased cytotoxicity due to the binding of a chimeric TCR expressed by an immune cell to the subject antigen. Such increased levels may be as compared to an un-activated control cell that does not express the chimeric TCR. In some instances, methods of the present disclosure result in a 10% or greater increase in cell killing of antigen expressing target cells as compared to un-activated control cells or the killing of cells not expressing the target antigen. In some instances, methods of the present disclosure result in a 10% or greater increase in cell killing, including but not limited to e.g., a 20% or greater increase, a 30% or greater increase, a 40% or greater increase, a 50% or greater increase, a 60% or greater increase, a 70% or greater increase, a 80% or greater increase, a 90% or greater increase, etc., as compared to an appropriate control.

Method of the present disclosure include methods of treating a subject for a condition. For example, in some instances, a subject may be treated for a condition by administering to the subject immune cells expressing a chimeric TCR as described herein. Subjects having a variety of different conditions may be treated according to the subject methods where such conditions will generally involve or be the result of one or more cell types that express an antigen to which a chimeric TCR may be directed. Accordingly, conditions that may be treated utilizing the instant methods include but are not limited to e.g., cancer where e.g., cells of the cancer express one or more antigens to which the chimeric TCR may be directed, infection where, e.g., infected cells express one or more antigens to which the chimeric TCR may be directed, and the like.

A variety of subjects are suitable for treatment with a subject method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent other than a chimeric TCR for the cancer and failed to respond to such treatment, or who has been treated with an agent other than a chimeric TCR for the cancer but relapsed after initial response to such treatment.

In some instances, methods of treatment utilizing one or more chimeric TCRs of the instant disclosure may find use in treating a cancer. Cancers, the treatment of which may include the use of one or more chimeric TCRs of the instant disclosure, will vary and may include but are not limited to e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

As discussed above, treatment methods of the present disclosure include treating a subject for a condition by administering to the subject an effective amount of the herein described nucleic acids encoding chimeric TCRs, vectors containing the subject nucleic acids, immune cells expressing the subject chimeric TCRs, and the like. Such conditions may, but need not necessarily, be, as noted above, cancer conditions.

An “effective amount” of an agent (including the subject nucleic acids, vectors, cells, etc.) is in some cases an amount that, when administered in one or more doses to an individual in need thereof results in a desirable pharmacological effect or biological response. In some cases, an effective amount of an agent, when administered in one or more doses to an individual in need thereof results in an increase in immune cell activation. In some cases, an effective amount of an agent, when administered in one or more doses to an individual in need thereof results in an increase of specific cell killing (cytotoxicity) of target cells expressing one or more antigens to which a subject chimeric TCR is directed.

An effective amount of cells may be formulated as a pharmaceutical composition. Pharmaceutical compositions may include a chimeric TCR expressing cell or a plurality of chimeric TCR expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are in some embodiments formulated for intravenous administration.

Pharmaceutical compositions may be administered in a manner appropriate to the disease to be treated. The quantity and frequency of administration may be determined by such factors as the condition of the patient, and the type and severity of the patient's disease, although appropriate dosages may be determined by clinical trials.

In some instances, a pharmaceutical composition that includes immune cells, such as chimeric TCR expressing immune cells of the present disclosure, may be administered at any appropriate dosage. Non-limiting examples of dosages that may be employed include but are not limited to e.g., dosages of 10⁴ to 10⁹ cells/kg body weight, including e.g., 10⁵ to 10⁶ cells/kg body weight, including all integer values within those ranges. Immune cell compositions may also be administered multiple times, including e.g., multiple times at the listed dosages. The subject immune cells may be administered by various routes including e.g., intravenous injection or infusion.

In some embodiments, nucleic acids encoding a chimeric TCR and/or cells expressing a chimeric TCR are administered in combination with a standard cancer therapy. For example, in some instances, nucleic acids encoding a chimeric TCR and/or cells expressing a chimeric TCR may be administered to induce immune cell activation and/or induce target cell killing with a course of treatment including one or more standard cancer therapies. In other instances nucleic acids encoding a chimeric TCR and/or cells expressing a chimeric TCR may be administered following one or more standard cancer therapies. In other instances, nucleic acids encoding a chimeric TCR and/or cells expressing a chimeric TCR may be administered during a standard cancer therapy.

Standard cancer therapies include surgery (e.g., surgical removal of cancerous tissue), radiation therapy, bone marrow transplantation, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin) , bevacizumab (Avastin™), cetuximab (Erbitux™), panitumumab (Vectibix™) Ipilimumab (Yervoy™), rituximab (Rituxan), alemtuzumab (Lemtrada™), Ofatumumab (Arzerra™) Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), 90Y-labelled ibritumomab tiuxetan (Zevalin™), 131I-labelled tositumoma (Bexxar™), etc. Suitable antibodies for use in cancer treatment include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y , VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, etc.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e g aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Patent No. 5,869,680; 6-thio derivatives described in WO 98/28288; sulfenamide derivatives described in U.S. Patent No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

In some instances, administration of chimeric TCR expressing immune cells may result in one or more side effects. Side effects associated with the administration of chimeric TCR expressing cells may include, but are not limited to cytokine release syndrome and hemophagocytic lymphohistiocytosis (Macrophage Activation Syndrome). In some instances, methods of treating a subject by administering chimeric TCR expressing immune cells may further include administration of one or more agents that reduce one or more side effects associated with the administration of the chimeric TCR expressing immune cells. Such agents include, but are not limited, to steroids, TNF-alpha inhibitors (e.g., entanercept), inhibitors of IL-6 (e.g., tocilizumab), and the like.

In some instances, methods of treating a subject by administering chimeric TCR expressing immune cells may further include administering an agent which enhances the activity of the treatment. Such agents that enhance the activity of the treatment will vary widely and may include but are not limited to e.g., agents that inhibit an inhibitor molecule. Suitable inhibitory molecules that may be targeted include but are not limited to e.g., PD1, PD-L1, CTLA4, TIM3, LAG3, VISTA, BTLA, TIGIT, LAIR1, CD160, 2B4 and TGFR beta.

Inhibiting of inhibitory molecules may be achieved by any convenient method including but not limited to e.g., the administration of a direct inhibitor of the inhibitory molecule (e.g., an antibody that binds the inhibitory molecule, a small molecule antagonist of the inhibitory molecule, etc.), administration of an agent that inhibits expression of the inhibitory molecule (e.g., an inhibitory nucleic acid, e.g., a dsRNA, e.g., an siRNA or shRNA targeting a nucleic acid encoding the inhibitory molecule), an indirect inhibitor of the inhibitory signaling, and the like. In some instances, an agent that may be administered may be an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy (Bristol-Myers Squibb)), Tremelimumab (Pfizer, formerly known as ticilimumab, CP-675,206)), TIM3, LAG3, or the like.

In some instances, cells expressing and/or transduced with nucleic acid encoding a chimeric TCR of the present disclosure may be administered to a subject alone or in combination with one or more additional agents. For example, for the treatment of a subject with cancer the subject may be administered an effective amount of immune cells expressing and/or transduced with nucleic acid encoding a chimeric TCR for treating the cancer and an additional therapy for treating the cancer (e.g., a chemotherapeutic, a therapeutic antibody for the treatment of cancer, CAR T cells, etc.). In some instances, immune cells expressing and/or transduced with nucleic acid encoding a chimeric TCR may be co-administered with immune cells expressing a CAR (e.g., CAR T cells). Where a subject is administered a chimeric TCR expressing cells and CAR expressing cells, the TCR expressing cells and CAR expressing cells may or may not target the same antigen. For example, a subject may be administered cells expressing or having nucleic acid encoding a chimeric TCR targeting a first antigen and cells expressing or having nucleic acid encoding a CAR targeting a second antigen, where the first and second antigens may be the same or different. Where employed in combination with a chimeric TCR of the present disclosure, a subject CAR may be configured to target essentially any antigen or bind any binding partner, including but not limited to e.g., any of the antigens and/or binding partners described herein.

Determining when combination therapies, e.g., involving the administration of one or more agents that ameliorates one or more side effects of a chimeric TCR immune cell therapy or involving the administration of one or more agents that enhances a chimeric TCR immune cell therapy, are indicated and the specifics of the administration of such combination therapies are within the skill of the relevant medical practitioner. In some instances, dosage regimens and treatment schedules of combination therapies may be determined through clinical trials.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered as below are provided. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A nucleic acid encoding a chimeric T cell antigen receptor (TCR) comprising a modified α-chain and a modified β-chain that, when present in an immune cell membrane, activates the immune cell when the chimeric TCR binds an antigen, wherein:
   a) the modified α-chain is a fusion polypeptide comprising a heterologous antigen-binding domain, that specifically binds the antigen, fused to the extracellular domain of a TCR α-chain; or
   b) the modified β-chain is a fusion polypeptide comprising a heterologous antigen-binding domain, that specifically binds the antigen, fused to the extracellular domain of a TCR β-chain.
2. The nucleic acid according to aspect 1, wherein the antigen is a cancer antigen.
3. The nucleic acid according to aspects 1 or 2, wherein the antigen is a cell surface antigen.
4. The nucleic acid according to aspects 1 or 2, wherein the antigen is a peptide-major histocompatibility complex (peptide-MHC).
5. The nucleic acid according to any of the preceding aspects, wherein the heterologous antigen-binding domain comprises an antibody.
6. The nucleic acid according to aspect 5, wherein the antibody is a scFv or a single domain antibody.
7. The nucleic acid according to any of aspects 1 to 3, wherein the heterologous antigen-binding domain comprises a ligand binding domain of a receptor.
8. The nucleic acid according to any of the preceding aspects, wherein the heterologous antigen-binding domain is fused directly to the extracellular domain.
9. The nucleic acid according to any of aspects 1 to 7, wherein the heterologous antigen-binding domain is fused to the extracellular domain by a linker.
10. The nucleic acid according to aspect 9, wherein the linker is less than 30 amino acids in length.
11. The nucleic acid according to aspect 10, wherein the linker is less than 20 amino acids in length.
12. The nucleic acid according to any of the preceding aspects, wherein the modified α-chain comprises a truncated α-chain, the modified β-chain comprises a truncated β-chain or the modified α-chain comprises a truncated α-chain and the modified β-chain comprises a truncated β-chain.
13. The nucleic acid according to aspect 12, wherein the modified α-chain, the modified β-chain or both the modified α-chain and the modified β-chain do not comprise a variable region.
14. The nucleic acid according to aspects 12 or 13, wherein the extracellular domain to which the heterologous antigen-binding domain is fused is a constant region of the TCR α-chain or the TCR β-chain.
15. The nucleic acid according to aspect 14, wherein the heterologous antigen-binding domain is fused directly to the constant region.
16. The nucleic acid according to aspect 14, wherein the heterologous antigen-binding domain is fused to the constant region by a linker.
17. The nucleic acid according to aspect 16, wherein the linker is less than 30 amino acids in length.
18. The nucleic acid according to aspect 17, wherein the linker is less than 20 amino acids in length.
19. The nucleic acid according to any of the preceding aspects, wherein the chimeric TCR comprises a recombinant disulfide bond between an α-chain cysteine mutation and a β-chain cysteine mutation.
20. The nucleic acid according to aspect 19, wherein the α-chain cysteine mutation is a T48C mutation and the β-chain cysteine mutation is a S57C mutation.
21. The nucleic acid according to any of the preceding aspects, wherein the modified α-chain and the modified β-chain are domain swapped modified α- and β-chains.
22. The nucleic acid according to aspect 21, wherein the domain swapped modified α- and β-chains comprise swapped α- and β-chain transmembrane regions.
23. The nucleic acid according to aspects 21 or 22, wherein the domain swapped modified α- and β-chains comprise swapped α- and β-chain cytoplasmic regions.
24. The nucleic acid according to any of aspects 21 to 23, wherein the domain swapped modified α- and β-chains comprise swapped α- and β-chain connecting regions.
25. The nucleic acid according to any of the preceding aspects, wherein the modified α-chain is a fusion polypeptide comprising two or more heterologous antigen-binding domains, that each specifically bind a different antigen, fused to the extracellular domain of a TCR α-chain.
26. The nucleic acid according to aspect 25, wherein the fusion polypeptide comprises a first heterologous antigen-binding domain fused to the extracellular domain of a TCR α-chain and a second heterologous antigen-binding domain fused to the first heterologous antigen-binding domain.
27. The nucleic acid according to any of the preceding aspects, wherein the modified β-chain is a fusion polypeptide comprising two or more heterologous antigen-binding domains, each of which specifically binds a different antigen, fused to the extracellular domain of a TCR β-chain.
28. The nucleic acid according to aspect 27, wherein the fusion polypeptide comprises a first heterologous antigen-binding domain fused to the extracellular domain of a TCR β-chain and a second heterologous antigen-binding domain fused to the first heterologous antigen-binding domain.
29. The nucleic acid according to any of the preceding aspects, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain.
30. The nucleic acid according to any of the preceding aspects, wherein the modified α-chain, the modified β-chain, or both the modified α-chain and the modified β-chain comprise a costimulatory domain.
31. The nucleic acid according to any of the preceding aspects, wherein the chimeric TCR activates the immune cell to exhibit cytotoxic activity to a target cell expressing the antigen.
32. The nucleic acid according to aspect 31, wherein the activated immune cell results in a 10% or greater increase in killing of the target cell as compared to a control immune cell without the chimeric TCR.
33. The nucleic acid according to any of the preceding aspects, wherein the modified α-chain and the modified β-chain are linked into a single chain by a linking polypeptide comprising a transmembrane domain.
34. A recombinant expression vector comprising the nucleic acid according to any of aspects 1 to 33, wherein the expression vector comprises a promoter operably linked to a nucleotide sequence encoding the modified α-chain and a nucleotide sequence encoding the modified β-chain.
35. The expression vector according to aspect 34, wherein the expression vector comprises a bicistronic-facilitating sequence between the nucleotide sequence encoding the modified α-chain and the nucleotide sequence encoding the modified β-chain.
36. The expression vector according to aspect 35, wherein the bicistronic-facilitating sequence comprises a furin cleavage site encoding sequence, an amino acid spacer encoding sequence and a 2A peptide encoding sequence.
37. The expression vector according to aspect 36, wherein the amino acid spacer encoding sequence comprises a nucleotide sequence encoding a V5 peptide.
38. The expression vector according to any of aspects 34 to 37, wherein the promoter is an inducible or conditional promoter.
39. A recombinant expression vector comprising the nucleic acid according to any of aspects 1 to 33, wherein the recombinant expression vector comprises a first promoter operably linked to a nucleotide sequence encoding the modified α-chain and a second promoter operably linked to a nucleotide sequence encoding the modified β-chain.
40. The expression vector according to aspect 39, wherein the first promoter is an inducible or conditional promoter.
41. The expression vector according to aspect 39 or 40, wherein the second promoter is an inducible or conditional promoter.
42. The expression vector according to any of aspects 39 to 41, wherein the first promoter and the second promoter are copies of the same promoter.
43. An immune cell comprising the expression vector according to any of aspects 34 to 42.
44. An immune cell genetically modified to comprise the nucleic acid according to any of aspects 1 to 33.
45. A method of killing a target cell, the method comprising contacting the target cell with the immune cell according to aspects 43 or 44, wherein the target cell expresses the antigen to which the chimeric TCR binds.
46. The method according to aspect 45, wherein the method is performed in vitro and the contacting comprises co-culturing the target cell and the immune cell.
47. The method according to aspect 45, wherein the method is performed in vivo and the contacting comprises administering the immune cell to a subject having the target cell.
48. The method according to aspect 47, wherein the target cell is a cancer cell and the method comprises administering to the subject an amount of the immune cells effective to treat the subject for the cancer.
49. A nucleic acid encoding a modified T cell antigen receptor (TCR) α-chain that, when present in a chimeric TCR within an immune cell membrane, activates the immune cell when the chimeric TCR binds an antigen, the modified TCR α-chain comprising:
   a heterologous antigen-binding domain;
   a truncated TCR α-chain extracellular domain linked to the heterologous antigen-binding domain;
   a TCR chain connecting region linked to the truncated TCR α-chain;
   a TCR chain transmembrane domain linked to the TCR chain connecting region; and
   a TCR chain cytoplasmic domain.
50. The nucleic acid according to aspect 49, wherein the antigen is a cancer antigen.
51. The nucleic acid according to aspects 49 or 50, wherein the antigen is a cell surface antigen.
52. The nucleic acid according to aspects 49 or 50, the antigen is a peptide-major histocompatibility complex (peptide-MHC).
53. The nucleic acid according to any of aspects 49 to 52, wherein the heterologous antigen-binding domain comprises an antibody.
54. The nucleic acid according to aspects 53, wherein the antibody is a scFv or a single domain antibody.
55. The nucleic acid according to any of aspects 49 to 51, wherein the heterologous antigen-binding domain comprises a ligand binding domain of a receptor.
56. The nucleic acid according to any of aspects 49 to 55, wherein the heterologous antigen-binding domain is linked directly to the truncated TCR α-chain extracellular domain.
57. The nucleic acid according to any of aspects 49 to 55, wherein the heterologous antigen-binding domain is linked to the truncated TCR α-chain extracellular domain by a linker.
58. The nucleic acid according to aspect 57, wherein the linker is less than 30 amino acids in length.
59. The nucleic acid according to aspects 58, wherein the linker is less than 20 amino acids in length.
60. The nucleic acid according to any of aspects 49 to 59, wherein the truncated TCR α-chain extracellular domain does not comprise a variable region.
61. The nucleic acid according to any of aspects 49 to 60, wherein the TCR chain connecting region comprises one or more cysteine substitutions.
62. The nucleic acid according to aspect 61, wherein the TCR chain connecting region is a TCR α-chain connecting region.
63. The nucleic acid according to aspect 62, wherein the one or more cysteine substitutions comprise a T48C mutation.
64. The nucleic acid according to aspect 61, wherein the TCR chain connecting region is a TCR β-chain connecting region.
65. The nucleic acid according to aspect 64, wherein the one or more cysteine substitutions comprise a S57C mutation.
66. The nucleic acid according to any of aspects 49 to 65, wherein the TCR chain transmembrane domain is a TCR α-chain transmembrane domain.
67. The nucleic acid according to any of aspects 49 to 65, wherein the TCR chain transmembrane domain is a TCR β-chain transmembrane domain.
68. The nucleic acid according to any of aspects 49 to 67, wherein the TCR chain cytoplasmic domain is a TCR α-chain cytoplasmic domain.
69. The nucleic acid according to any of aspects 49 to 68, wherein the TCR chain cytoplasmic domain is a TCR β-chain cytoplasmic domain.
70. The nucleic acid according to any of aspects 49 to 69, wherein the modified TCR α-chain comprises two different heterologous antigen-binding domains.
71. The nucleic acid according to any of aspects 49 to 70, wherein the modified TCR α-chain further comprises a costimulatory domain.
72. The nucleic acid according to any of aspects 49 to 71, wherein the chimeric TCR comprising the modified TCR α-chain activates the immune cell to exhibit cytotoxic activity to a target cell expressing the antigen.
73. The nucleic acid according to aspect 72,wherein the activated immune cell results in a 10% or greater increase in killing of the target cell as compared to a control immune cell without the chimeric TCR.
74. A recombinant expression vector comprising the nucleic acid according to any of aspects 49 to 73.
75. An immune cell comprising the expression vector of aspect 74.
76. An immune cell genetically modified to comprise the nucleic acid according to any of aspects 49 to 73.
77. An immune cell comprising:
   a first nucleic acid encoding a modified TCR α-chain comprising:
      a heterologous antigen-binding domain linked to a TCR α-chain; and
   a first cysteine substitution within the chain connecting region of the TCR α-chain; and
   a second nucleic acid encoding a modified TCR β-chain comprising a second cysteine substitution, wherein the first and second cysteine substitutions result in a recombinant disulfide bond between the modified TCR α-chain and the modified TCR β-chain.
78. The immune cell according to aspect 77, wherein the first cysteine substitution is a T48C mutation and the second cysteine substitution is a S57C mutation.
79. A method of killing a target cell, the method comprising contacting the target cell with an immune cell according to any of aspects 75 to 78, wherein the target cell expresses the antigen to which the chimeric TCR binds.
80. The method according to aspect 79, wherein the method is performed in vitro and the contacting comprises co-culturing the target cell and the immune cell.
81. The method according to aspect 79, wherein the method is performed in vivo and the contacting comprises administering the immune cell to a subject having the target cell.
82. The method according to aspect 81, wherein the target cell is a cancer cell and the method comprises administering to the subject an amount of the immune cells effective to treat the subject for the cancer.
83. A nucleic acid encoding a modified T cell antigen receptor (TCR) β-chain that, when present in a chimeric TCR within an immune cell membrane, activates the immune cell when the chimeric TCR binds an antigen, the modified TCR β-chain comprising:
   a heterologous antigen-binding domain;
   a truncated TCR β-chain extracellular domain linked to the heterologous antigen-binding domain;
   a TCR chain connecting region linked to the truncated TCR β-chain;
   a TCR chain transmembrane domain linked to the TCR chain connecting region; and
   a TCR chain cytoplasmic domain.
84. The nucleic acid according to aspect 83, wherein the antigen is a cancer antigen.
85. The nucleic acid according to aspects 83 or 84, wherein the antigen is a cell surface antigen.
86. The nucleic acid according to aspects 83 or 84, the antigen is a peptide-major histocompatibility complex (peptide-MHC).
87. The nucleic acid according to any of aspects 83 to 86, wherein the heterologous antigen-binding domain comprises an antibody.
88. The nucleic acid according to any of aspect 87, wherein the antibody is a scFv or a single domain antibody.
89. The nucleic acid according to any of aspects 83 to 85, wherein the heterologous antigen-binding domain comprises a ligand binding domain of a receptor.
90. The nucleic acid according to any of aspects 83 to 89, wherein the heterologous antigen-binding domain is linked directly to the truncated TCR β-chain extracellular domain.
91. The nucleic acid according to any of aspects 83 to 89, wherein the heterologous antigen-binding domain is linked to the truncated TCR β-chain extracellular domain by a linker.
92. The nucleic acid according to aspect 91, wherein the linker is less than 30 amino acids in length.
93. The nucleic acid according to aspect 92, wherein the linker is less than 20 amino acids in length.
94. The nucleic acid according to any of aspects 83 to 93, wherein the truncated TCR β-chain extracellular domain does not comprise a variable region.
95. The nucleic acid according to any of aspects 83 to 94, wherein the TCR chain connecting region comprises one or more cysteine substitutions.
96. The nucleic acid according to aspect 95, wherein the TCR chain connecting region is a TCR β-chain connecting region.
97. The nucleic acid according to aspect 96, wherein the one or more cysteine substitutions comprise a S57C mutation.
98. The nucleic acid according to aspect 95, wherein the TCR chain connecting region is a TCR α-chain connecting region.
99. The nucleic acid according to aspect 98, wherein the one or more cysteine substitutions comprise a T48C mutation.
100. The nucleic acid according to any of aspects 83 to 99, wherein the TCR chain transmembrane domain is a TCR β-chain transmembrane domain.
101. The nucleic acid according to any of aspects 83 to 99, wherein the TCR chain transmembrane domain is a TCR α-chain transmembrane domain.
102. The nucleic acid according to any of aspects 83 to 101, wherein the TCR chain cytoplasmic domain is a TCR β-chain cytoplasmic domain.
103. The nucleic acid according to any of aspects 83 to 101, wherein the TCR chain cytoplasmic domain is a TCR α-chain cytoplasmic domain.
104. The nucleic acid according to any of aspects 83 to 103, wherein the modified TCR β-chain comprises two different heterologous antigen-binding domains.
105. The nucleic acid according to any of aspects 83 to 104, wherein the modified TCR β-chain further comprises a costimulatory domain.
106. The nucleic acid according to any of aspects 83 to 105, wherein the chimeric TCR comprising the modified TCR β-chain activates the immune cell to exhibit cytotoxic activity to a target cell expressing the antigen.
107. The nucleic acid according to aspect 106, wherein the activated immune cell results in a 10% or greater increase in killing of the target cell as compared to a control immune cell without the chimeric TCR.
108. A recombinant expression vector comprising the nucleic acid according to any of aspects 83 to 107.
109. An immune cell comprising the expression vector of aspect 108.
110. An immune cell genetically modified to comprise the nucleic acid according to any of aspects 83 to 107.
111. An immune cell comprising:
   a first nucleic acid encoding a modified TCR β-chain comprising:
      a heterologous antigen-binding domain linked to a TCR β-chain; and
   a first cysteine substitution within the chain connecting region of the TCR β-chain; and
   a second nucleic acid encoding a modified TCR α-chain comprising a second cysteine substitution, wherein the first and second cysteine substitutions result in a recombinant disulfide bond between the modified TCR β-chain and the modified TCR α-chain.
112. The immune cell according to aspect 111, wherein the first cysteine substitution is a S57C mutation and the second cysteine substitution is a T48C mutation.
113. A method of killing a target cell, the method comprising contacting the target cell with an immune cell according to any of aspects 109 to 112, wherein the target cell expresses the antigen to which the chimeric TCR binds.
114. The method according to aspect 113, wherein the method is performed in vitro and the contacting comprises co-culturing the target cell and the immune cell.
115. The method according to aspect 113, wherein the method is performed in vivo and the contacting comprises administering the immune cell to a subject having the target cell.
116. The method according to aspect 115, wherein the target cell is a cancer cell and the method comprises administering to the subject an amount of the immune cells effective to treat the subject for the cancer.
117. A method of treating a subject for a condition, the method comprising:
   administering to the subject an effective amount of the immune cells according to any of aspects 43, 44, 75-78 and 109-112 in combination with an agent that ameliorates at least one side effect of the immune cells.
118. The method according to aspect 117, wherein the condition is cancer.
119. A method of treating a subject for cancer, the method comprising:
   administering to the subject an effective amount of the immune cells according to any of aspects 43, 44, 75-78 and 109-112 in combination with a conventional cancer therapy.
120. The method according to aspect 119, wherein the immune cells and the conventional cancer therapy are administered in combination with an agent that ameliorates at least one side effect of the immune cells.
121. A chimeric T cell antigen receptor (TCR) comprising a modified α-chain and a modified β-chain that, when present in an immune cell membrane, activates the immune cell when the chimeric TCR binds an antigen, wherein:
   a) the modified α-chain is a fusion polypeptide comprising a heterologous antigen-binding domain, that specifically binds the antigen, fused to the extracellular domain of a TCR α-chain; or
   b) the modified β-chain is a fusion polypeptide comprising a heterologous antigen-binding domain, that specifically binds the antigen, fused to the extracellular domain of a TCR β-chain.
122. The chimeric TCR according to aspect 121, wherein the antigen is a cancer antigen.
123. The chimeric TCR according to aspects 121 or 122, wherein the antigen is a cell surface antigen.
124. The chimeric TCR according to aspects 121 or 122, wherein the antigen is a peptide-major histocompatibility complex (peptide-MHC).
125. The chimeric TCR according to any of aspects 121 to 124, wherein the heterologous antigen-binding domain comprises an antibody.
126. The chimeric TCR according to aspect 125, wherein the antibody is a scFv or a single domain antibody.
127. The chimeric TCR according to any of aspects 121 to 123, wherein the heterologous antigen-binding domain comprises a ligand binding domain of a receptor.
128. The chimeric TCR according to any of aspects 121 to 127, wherein the heterologous antigen-binding domain is fused directly to the extracellular domain.
129. The chimeric TCR according to any of aspects 121 to 127, wherein the heterologous antigen-binding domain is fused to the extracellular domain by a linker.
130. The chimeric TCR according to aspect 129, wherein the linker is less than 30 amino acids in length.
131. The chimeric TCR according to aspect 130, wherein the linker is less than 20 amino acids in length.
132. The chimeric TCR according to any of aspects 121 to 131, wherein the modified α-chain comprises a truncated α-chain, the modified β-chain comprises a truncated β-chain or the modified α-chain comprises a truncated α-chain and the modified β-chain comprises a truncated β-chain.
133. The chimeric TCR according to aspect 132, wherein the modified α-chain, the modified β-chain or both the modified α-chain and the modified β-chain do not comprise a variable region.
134. The chimeric TCR according to aspects 132 or 133, wherein the extracellular domain to which the heterologous antigen-binding domain is fused is a constant region of the TCR α-chain or the TCR β-chain.
135. The chimeric TCR according to aspect 134, wherein the heterologous antigen-binding domain is fused directly to the constant region.
136. The chimeric TCR according to aspect 134, wherein the heterologous antigen-binding domain is fused to the constant region by a linker.
137. The chimeric TCR according to aspect 136, wherein the linker is less than 30 amino acids in length.
138. The chimeric TCR according to aspect 137, wherein the linker is less than 20 amino acids in length.
139. The chimeric TCR according to any of aspects 121 to 138, wherein the chimeric TCR comprises a recombinant disulfide bond between a α-chain cysteine mutation and a β-chain cysteine mutation.
140. The chimeric TCR according to aspect 139, wherein the α-chain cysteine mutation is a T48C mutation and the β-chain cysteine mutation is a S57C mutation.
141. The chimeric TCR according to any of aspects 121 to 140, wherein the modified α-chain and the modified β-chain are domain swapped modified α- and β-chains.
142. The chimeric TCR according to aspect 141, wherein the domain swapped modified α- and β-chains comprise swapped α- and β-chain transmembrane regions.
143. The chimeric TCR according to aspects 141 or 142, wherein the domain swapped modified α- and β-chains comprise swapped α- and β-chain cytoplasmic regions.
144. The chimeric TCR according to any of aspects 141 to 143, wherein the domain swapped modified α- and β-chains comprise swapped α- and β-chain connecting regions.
145. The chimeric TCR according to any of aspects 121 to 144, wherein the modified α-chain is a fusion polypeptide comprising two or more heterologous antigen-binding domains, that each specifically bind a different antigen, fused to the extracellular domain of a TCR α-chain.
146. The chimeric TCR according to aspect 145, wherein the fusion polypeptide comprises a first heterologous antigen-binding domain fused to the extracellular domain of a TCR α-chain and a second heterologous antigen-binding domain fused to the first heterologous antigen-binding domain.
147. The chimeric TCR according to any of aspects 121 to 146, wherein the modified β-chain is a fusion polypeptide comprising two or more heterologous antigen-binding domains, that each specifically bind a different antigen, fused to the extracellular domain of a TCR β-chain.
148. The chimeric TCR according to aspect 147, wherein the fusion polypeptide comprises a first heterologous antigen-binding domain fused to the extracellular domain of a TCR β-chain and a second heterologous antigen-binding domain fused to the first heterologous antigen-binding domain.
149. The chimeric TCR according to any of aspects 121 to 148, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain.
150. The chimeric TCR according to any of aspects 121 to 149, wherein the modified α-chain, the modified β-chain, or both the modified α-chain and the modified β-chain comprise a costimulatory domain.
151. The chimeric TCR according to any of aspects 121 to 150, wherein the chimeric TCR activates the immune cell to exhibit cytotoxic activity to a target cell expressing the antigen.
152. The chimeric TCR according to aspect 151, wherein the activated immune cell results in a 10% or greater increase in killing of the target cell as compared to a control immune cell without the chimeric TCR.
153. The chimeric TCR according to any of aspects 121 to 152, wherein the modified α-chain and the modified β-chain are linked into a single chain by a linking polypeptide comprising a transmembrane domain.
154. A method of killing a target cell, the method comprising contacting the target cell with an immune cell expressing a chimeric TCR according to any of aspects 149 to 153, wherein the modified α-chain comprises a heterologous antigen-binding domain specific for a first antigen expressed by the target cell and the modified β-chain comprises a heterologous antigen-binding domain specific for a second antigen expressed by the target cell.
155. The method according to aspect 154, wherein the first antigen expressed by the target cell and the second antigen expressed by the target cell are the same antigen.
156. The method according to aspect 155, wherein the heterologous antigen-binding domain of the modified α-chain and the heterologous antigen-binding domain of the modified β-chain are the same heterologous antigen-binding domain.
157. The method according to aspect 155, wherein the heterologous antigen-binding domain of the modified α-chain and the heterologous antigen-binding domain of the modified β-chain are different heterologous antigen-binding domains.
158. The method according to aspect 154, wherein the first antigen expressed by the target cell and the second antigen expressed by the target cell are different antigens.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Engineered T Cell Antigen Receptor (TCR) Constructs with Redirected Antigen Binding

Receptor molecule platforms and methods have been developed that when expressed in a T cell can allow the T cell to detect and respond to antigens present on the surface of a target cell. T cells naturally express T cell receptors (TCRs) that mediate recognition of pathogenic peptides presented in the context of an MHC molecule. TCRs are heterodimers made up of an alpha chain and a beta chain, and the TCR complex is composed of the TCR alpha and beta chains together with three dimers of CD3 chains (CD3δ/ε, CD3γ/ε, CD3δ/δ). The TCR chains recognize and bind a cognate peptide-MHC antigen, and the CD3 chains provide the signaling modules that induce T cell activation upon antigen binding. T cells with naturally occurring tumor-reactive TCRs, as well as those genetically modified to express engineered tumor-reactive TCRs, have been successfully used to treat patients with a diverse range of cancers.

As an alternative approach, chimeric antigen receptors (CARs) are another receptor platform used to engineer tumor-reactive T cells. CARs combine the following domains: 1) a variable extracellular recognition domain (e.g. an scFv for an antigen), 2) a hinge/transmembrane domain, 3) intracellular signaling domains, including TCR complex signaling domains such as ITAMs, and potentially co-stimulatory domains. The vast majority of CAR designs include the cytoplasmic portion of the CD3 chain as the main signaling component, and later generation designs also incorporate co-stimulatory domains. Thus, while CARs incorporate some signaling domains that naturally occur in the TCR complex, CARs do not include the majority of the signaling domains found in the TCR complex, such as domains from CD3δ/ε/γ.

Unlike TCRs, CARs typically bind surface antigens via their extracellular recognition domain Given that the TCR complex has signaling capabilities not found in CARs and that CARs are able to recognize surface antigens that are inaccessible to TCRs, combining the targeting ability of CARs (e.g. targeting any surface antigen that has a characterized specific binding domain) with the highly evolved signaling capacity of the TCR complex was pursued.

To this purpose engineered TCRs (fusion molecules also terms synthetic TCRs “synTCR”) having redirected antigen binding, e.g., one or more antibody domains linked extracellularly to a portion of one or more of the TCR chains, were designed. As a proof-of-principle approach anti-GFP nanobody based and/or anti-mesothelin scFv based antigen binding domains were fused to the alpha and/or beta chains of engineered TCRs.

Developed constructs include the following:

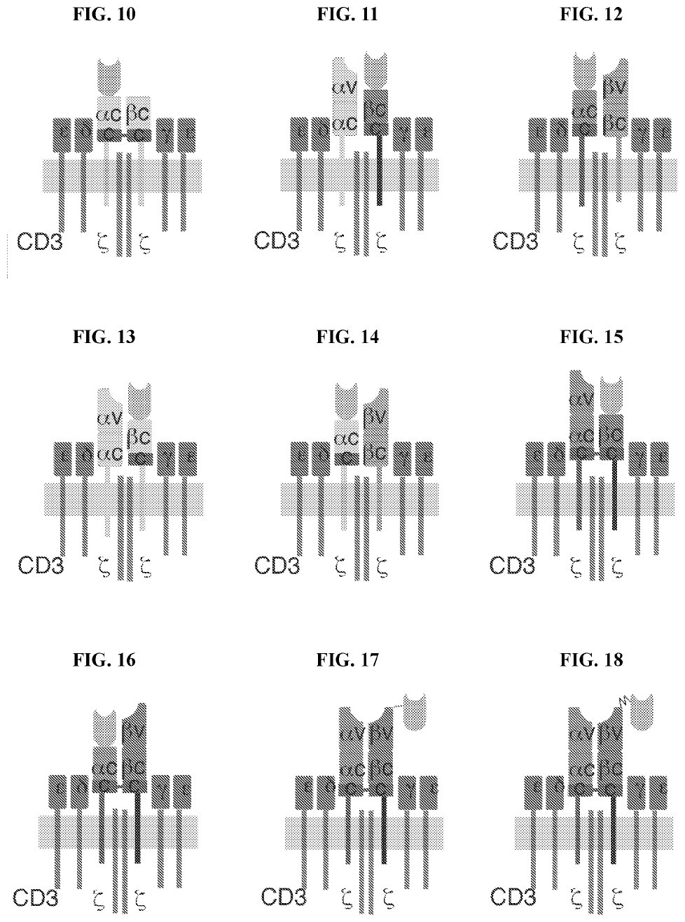

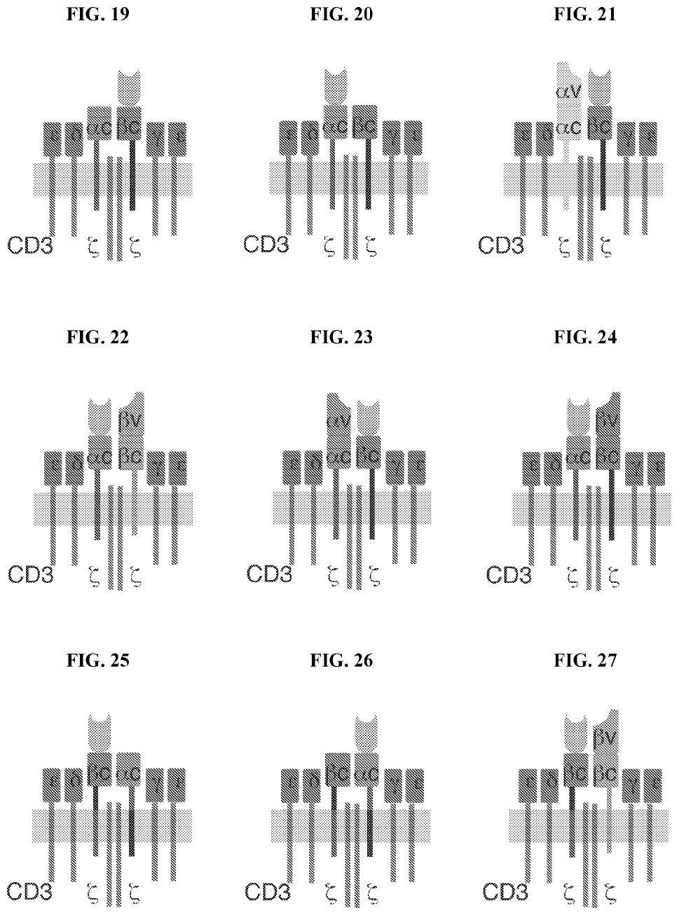

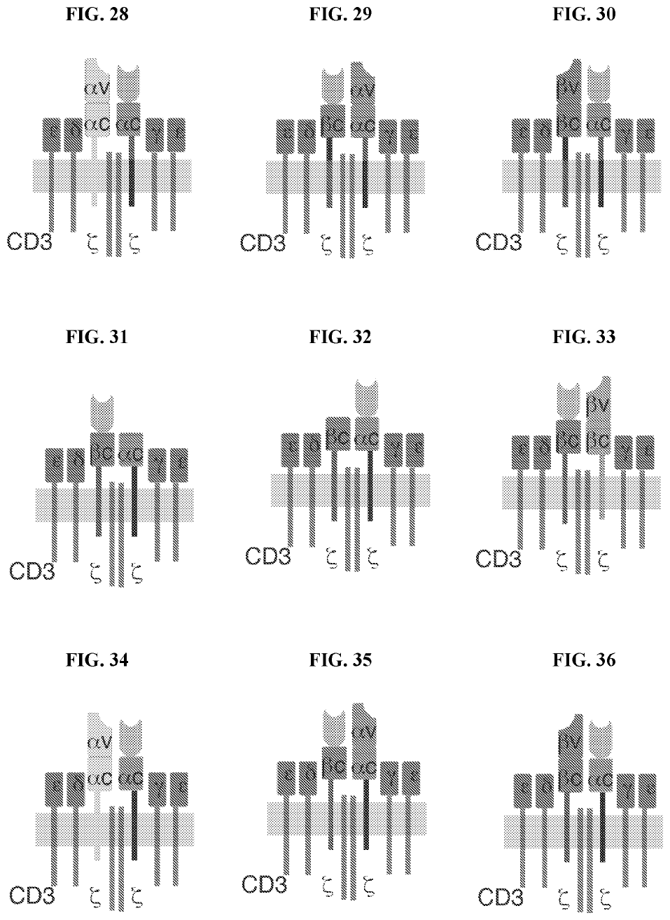

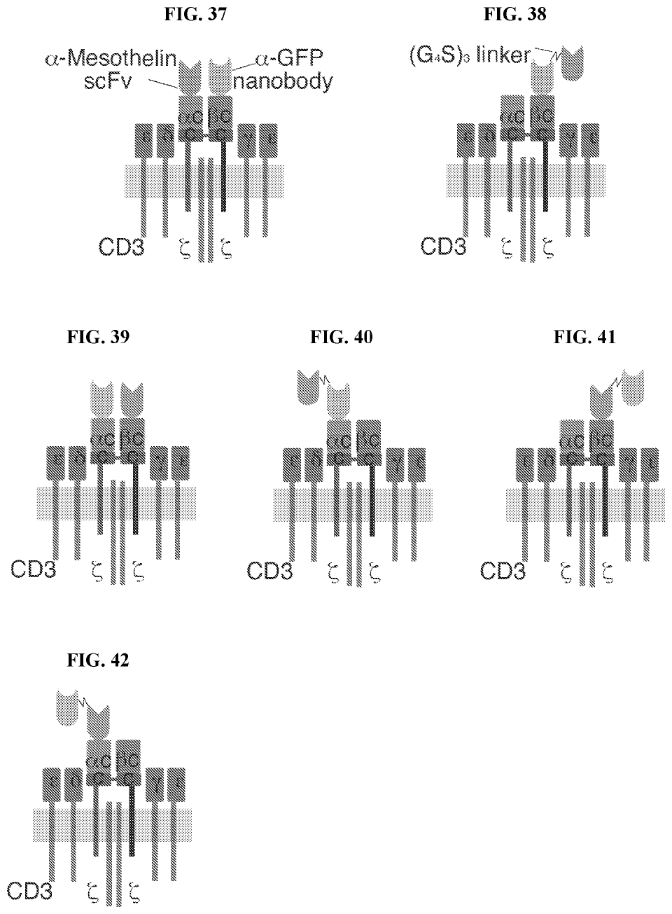

LaG17_AggenLink_IG4_TCR(P145), as depicted in FIG. 5 and having the following translated
amino acid sequence:
(SEQ ID NO: 90)
MADVQLVESGGGLVQAGGSLRLSCAASGRTISMAAMSWFRQAPGKEREFVAGISRSAGSAVH
ADSVKGRFTISRDNTKNTLYLQMNSLKAEDTAVYYCAVRTSGFFGSIPRTGTAFDYWGQGTQV
TVSGSADDAKKDAAKKDGKSMSIGLLCCAALSLLWAGPVNAGVTQTPKFQVLKTGQSMTLQC
AQDMNHEYMSWYRQDPGMGLRLIHYSVGAGITDQGEVPNGYNVSRSTTEDFPLRLLSAAPSQT
SVYFCASSYVGNTGELFFGEGSRLTVLEDLNKVFPPEVAVFEPSEAEISHTQKATLVCLATGFFP
DHVELSWWVNGKEVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYEILLGKATLYAVLV
SALVLMAMVKRKDFRKRRGKPIPNPLLGLDSTSGSGEGRGSLLTCGDVEENPGPMETLLGLLIL
WLQLQWVSSKQEVTQIPAALSVPEGENLVLNCSFTDSAIYNLQWFRQDPGKGLTSLLLIQSSQR
EQTSGRLNASLDKSSGRSTLYIAASQPGDSATYLCAVRPTSGGSYIPTFGRGTSLIVHPPNIQNPD
PAVYQLRDSKSSDKSVCLFTDFDSQTNVSQSKDSDVYITDKTVLDMRSMDFKSNSAVAWSNKS
DFACANAFNNSIIPEDTFFPSPESSCDVKLVEKSFETDTNLNFQNLSVIGFRILLLKVAGFNLLMTL
RLWSS.
LaG17_(G4S)3_IG4_TCR(P146), as depicted in FIG. 6 and having the following translated
amino acid sequence:
(SEQ ID NO: 91)
MADVQLVESGGGLVQAGGSLRLSCAASGRTISMAAMSWFRQAPGKEREFVAGISRSAGSAVH
ADSVKGRFTISRDNTKNTLYLQMNSLKAEDTAVYYCAVRTSGFFGSIPRTGTAFDYWGQGTQV
TVSGGGGSGGGGSGGGGSMSIGLLCCAALSLLWAGPVNAGVTQTPKFQVLKTGQSMTLQCAQ
DMNHEYMSWYRQDPGMGLRLIHYSVGAGITDQGEVPNGYNVSRSTTEDFPLRLLSAAPSQTSV
YFCASSYVGNTGELFFGEGSRLTVLEDLNKVFPPEVAVFEPSEAEISHTQKATLVCLATGFFPDH
VELSWWVNGKEVHSGVSTDPQPLKEQPALNDSRYCLSSRLRVSATFWQNPRNHFRCQVQFYG
LSENDEWTQDRAKPVTQIVSAEAWGRADCGFTSVSYQQGVLSATILYEILLGKATLYAVLVSAL
VLMAMVKRKDFRKRRGKPIPNPLLGLDSTSGSGEGRGSLLTCGDVEENPGPMETLLGLLILWLQ
LQWVSSKQEVTQIPAALSVPEGENLVLNCSFTDSAIYNLQWFRQDPGKGLTSLLLIQSSQREQTS
GRLNASLDKSSGRSTLYIAASQPGDSATYLCAVRPTSGGSYIPTFGRGTSLIVHPPNIQNPDPAVY
QLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLW
SS.
LaG17-TRBC1_TRAC (P147) as depicted in FIG. 7 and having the following translated amino
acid sequence:
(SEQ ID NO: 92)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVCTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDFRKRRGKPIPNPLLGLDSTSGSGEGRGSLLTCGDVEENPGPMALPV
TALLLPLALLLHAARPYPYDVPDYAPNIQNPDPAVYQLRDSKSSDKSVCLFTDFDSQTNVSQSK
DSDVYITDKCVLDMRSMDFKSNSAVAWSNKSDFACANAFNNSIIPEDTFFPSPESSCDVKLVEKS
FETDTNLNFQNLSVIGFRILLLKVAGFNLLMTLRLWSS.
TRBC1_LaG17-TRAC (P148) as depicted in FIG. 8 and having the following translated amino
acid sequence:
(SEQ ID NO: 93)
MALPVTALLLPLALLLHAARPYPYDVPDYAEDLNKVFPPEVAVFEPSEAEISHTQKATLVCLAT
GFFPDHVELSWWVNGKEVHSGVCTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPEQKLISEEDLMADVQLVESGGGLVQAGGSLRLSCAASGRTISMAAMSW
FRQAPGKEREFVAGISRSAGSAVHADSVKGRFTISRDNTKNTLYLQMNSLKAEDTAVYYCAVR
TSGFFGSIPRTGTAFDYWGQGTQVTVSPNIQNPDPAVYQLRDSKSSDKSVCLFTDFDSQTNVSQS
KDSDVYITDKCVLDMRSMDFKSNSAVAWSNKSDFACANAFNNSIIPEDTFFPSPESSCDVKLVE
KSFETDTNLNFQNLSVIGFRILLLKVAGFNLLMTLRLWSS.
LaG17-muTCB1_muTCRA (P149), as depicted in FIG. 9 and having the following translated
amino acid sequence:
(SEQ ID NO: 94)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLRNVTPPKVSLFEPSKAEIANKQKATLVCL
ARGFFPDHVELSWWVNGKEVHSGVSTDPQAYKESNYSYCLSSRLRVCATFWHNPRNHFRCQV
QFHGLSEEDKWPEGSPKPVTQNISAEAWGRADCGITSASYQQGVLSATILYEILLGKATLYAVL
VSTLVVMAMVKRKNSRKRRGKPIPNPLLGLDSTSGSGEGRGSLLTCGDVEENPGPMALPVTAL
LLPLALLLHAARPYPYDVPDYAPYIQNPEPAVYQLKDPRSQDSTLCLFTDFDSQINVPKTMESGT
FITDKTVLDMKAMDSKSNGAIAWSNQTSFTCQDIFKETNACYPSSDVPCDATLTEKSFETDMNL
NFQNLSVMGLRILLLKVAGFNLLMTLRLWSS.
muTCB1_LaG17-muTCRA (P150), as depicted in FIG. 10 and having the following translated
amino acid sequence:
(SEQ ID NO: 95)
MALPVTALLLPLALLLHAARPYPYDVPDYAEDLRNVTPPKVSLFEPSKAEIANKQKATLVCLAR
GFFPDHVELSWWVNGKEVHSGVSTDPQAYKESNYSYCLSSRLRVCATFWHNPRNHFRCQVQF
HGLSEEDKWPEGSPKPVTQNISAEAWGRADCGITSASYQQGVLSATILYEILLGKATLYAVLVST
LVVMAMVKRKNSRKRRGKPIPNPLLGLDSTSGSGEGRGSLLTCGDVEENPGPMALPVTALLLPL
ALLLHAARPEQKLISEEDLMADVQLVESGGGLVQAGGSLRLSCAASGRTISMAAMSWFRQAPG
KEREFVAGISRSAGSAVHADSVKGRFTISRDNTKNTLYLQMNSLKAEDTAVYYCAVRTSGFFGS
IPRTGTAFDYWGQGTQVTVSPYIQNPEPAVYQLKDPRSQDSTLCLFTDFDSQINVPKTMESGTFI
TDKTVLDMKAMDSKSNGAIAWSNQTSFTCQDIFKETNACYPSSDVPCDATLTEKSFETDMNLN
FQNLSVMGLRILLLKVAGFNLLMTLRLWSS.
pHR_LaG17-TRBC1 (P176), as depicted in FIG. 11 and having the following translated amino
acid sequence:
(SEQ ID NO: 96)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVCTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDF.
pHR_LaG17-TRAC (P177), as depicted in FIG. 12 and having the following translated amino
acid sequence:
(SEQ ID NO: 97)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSPNIQNPDPAVYQLRDSKSSDKSVCLFTDFDSQ
TNVSQSKDSDVYITDKCVLDMRSMDFKSNSAVAWSNKSDFACANAFNNSIIPEDTFFPSPESSCD
VKLVEKSFETDTNLNFQNLSVIGFRILLLKVAGFNLLMTLRLWSS.
pHR_LaG17-muTCB1 (P178), as depicted in FIG. 13 and having the following translated
amino acid sequence:
(SEQ ID NO: 98)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLRNVTPPKVSLFEPSKAEIANKQKATLVCL
ARGFFPDHVELSWWVNGKEVHSGVSTDPQAYKESNYSYCLSSRLRVCATFWHNPRNHFRCQV
QFHGLSEEDKWPEGSPKPVTQNISAEAWGRADCGITSASYQQGVLSATILYEILLGKATLYAVL
VSTLVVMAMVKRKNS.
pHR_LaG17-muTCRA (P179), as depicted in FIG. 14 and having the following translated
amino acid sequence:
(SEQ ID NO: 99)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSPYIQNPEPAVYQLKDPRSQDSTLCLFTDFDSQI
NVPKTMESGTFITDKTVLDMKAMDSKSNGAIAWSNQTSFTCQDIFKETNACYPSSDVPCDATLT
EKSFETDMNLNFQNLSVMGLRILLLKVAGFNLLMTLRLWSS.
pHR_LaG17-TRBC1_IG4av-TRAC (P180), as depicted in FIG. 15 and having the following
translated amino acid sequence:
(SEQ ID NO: 100)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVCTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDFRKRRGKPIPNPLLGLDSTSGSGEGRGSLLTCGDVEENPGPMALPV
TALLLPLALLLHAARPYPYDVPDYAMETLLGLLILWLQLQWVSSKQEVTQIPAALSVPEGENLV
LNCSFTDSAIYNLQWFRQDPGKGLTSLLLIQSSQREQTSGRLNASLDKSSGRSTLYIAASQPGDS
ATYLCAVRPTSGGSYIPTFGRGTSLIVHPPNIQNPDPAVYQLRDSKSSDKSVCLFTDFDSQTNVSQ
SKDSDVYITDKCVLDMRSMDFKSNSAVAWSNKSDFACANAFNNSIIPEDTFFPSPESSCDVKLVE
KSFETDTNLNFQNLSVIGFRILLLKVAGFNLLMTLRLWSS.
pHR_IG4bv-TRBC1_LaG17-TRAC (P181), as depicted in FIG. 16 and having the following
translated amino acid sequence:
(SEQ ID NO: 101)
MALPVTALLLPLALLLHAARPYPYDVPDYAMSIGLLCCAALSLLWAGPVNAGVTQTPKFQVLK
TGQSMTLQCAQDMNHEYMSWYRQDPGMGLRLIHYSVGAGITDQGEVPNGYNVSRSTTEDFPL
RLLSAAPSQTSVYFCASSYVGNTGELFFGEGSRLTVLEDLNKVFPPEVAVFEPSEAEISHTQKATL
VCLATGFFPDHVELSWWVNGKEVHSGVCTDPQPLKEQPALNDSRYCLSSRLRVSATFWQNPRN
HFRCQVQFYGLSENDEWTQDRAKPVTQIVSAEAWGRADCGFTSVSYQQGVLSATILYEILLGK
ATLYAVLVSALVLMAMVKRKDFRKRRGKPIPNPLLGLDSTSGSGEGRGSLLTCGDVEENPGPM
ALPVTALLLPLALLLHAARPEQKLISEEDLMADVQLVESGGGLVQAGGSLRLSCAASGRTISMA
AMSWFRQAPGKEREFVAGISRSAGSAVHADSVKGRFTISRDNTKNTLYLQMNSLKAEDTAVYY
CAVRTSGFFGSIPRTGTAFDYWGQGTQVTVSPNIQNPDPAVYQLRDSKSSDKSVCLFTDFDSQT
NVSQSKDSDVYITDKCVLDMRSMDFKSNSAVAWSNKSDFACANAFNNSIIPEDTFFPSPESSCD
VKLVEKSFETDTNLNFQNLSVIGFRILLLKVAGFNLLMTLRLWSS.
pHR_LaG17_AggenLink_IG4_TCR_CysteineMod (P189), as depicted in FIG. 17 and having
the following translated amino acid sequence:
(SEQ ID NO: 102)
MADVQLVESGGGLVQAGGSLRLSCAASGRTISMAAMSWFRQAPGKEREFVAGISRSAGSAVH
ADSVKGRFTISRDNTKNTLYLQMNSLKAEDTAVYYCAVRTSGFFGSIPRTGTAFDYWGQGTQV
TVSGSADDAKKDAAKKDGKSMSIGLLCCAALSLLWAGPVNAGVTQTPKFQVLKTGQSMTLQC
AQDMNHEYMSWYRQDPGMGLRLIHYSVGAGITDQGEVPNGYNVSRSTTEDFPLRLLSAAPSQT
SVYFCASSYVGNTGELFFGEGSRLTVLEDLNKVFPPEVAVFEPSEAEISHTQKATLVCLATGFFP
DHVELSWWVNGKEVHSGVCTDPQPLKEQPALNDSRYCLSSRLRVSATFWQNPRNHFRCQVQF
YGLSENDEWTQDRAKPVTQIVSAEAWGRADCGFTSVSYQQGVLSATILYEILLGKATLYAVLV
SALVLMAMVKRKDFRKRRGKPIPNPLLGLDSTSGSGEGRGSLLTCGDVEENPGPMETLLGLLIL
WLQLQWVSSKQEVTQIPAALSVPEGENLVLNCSFTDSAIYNLQWFRQDPGKGLTSLLLIQSSQR
EQTSGRLNASLDKSSGRSTLYIAASQPGDSATYLCAVRPTSGGSYIPTFGRGTSLIVHPPNIQNPD
PAVYQLRDSKSSDKSVCLFTDFDSQTNVSQSKDSDVYITDKCVLDMRSMDFKSNSAVAWSNKS
DFACANAFNNSIIPEDTFFPSPESSCDVKLVEKSFETDTNLNFQNLSVIGFRILLLKVAGFNLLMTL
RLWSS.
pHR_LaG17_(G4S)3_IG4_TCR_CysteineMod (P190), as depicted in FIG. 18 and having the
following translated amino acid sequence:
(SEQ ID NO: 103)
MADVQLVESGGGLVQAGGSLRLSCAASGRTISMAAMSWFRQAPGKEREFVAGISRSAGSAVH
ADSVKGRFTISRDNTKNTLYLQMNSLKAEDTAVYYCAVRTSGFFGSIPRTGTAFDYWGQGTQV
TVSGGGGSGGGGSGGGGSMSIGLLCCAALSLLWAGPVNAGVTQTPKFQVLKTGQSMTLQCAQ
DMNHEYMSWYRQDPGMGLRLIHYSVGAGITDQGEVPNGYNVSRSTTEDFPLRLLSAAPSQTSV
YFCASSYVGNTGELFFGEGSRLTVLEDLNKVFPPEVAVFEPSEAEISHTQKATLVCLATGFFPDH
VELSWWVNGKEVHSGVCTDPQPLKEQPALNDSRYCLSSRLRVSATFWQNPRNHFRCQVQFYG
LSENDEWTQDRAKPVTQIVSAEAWGRADCGFTSVSYQQGVLSATILYEILLGKATLYAVLVSAL
VLMAMVKRKDFRKRRGKPIPNPLLGLDSTSGSGEGRGSLLTCGDVEENPGPMETLLGLLILWLQ
LQWVSSKQEVTQIPAALSVPEGENLVLNCSFTDSAIYNLQWFRQDPGKGLTSLLLIQSSQREQTS
GRLNASLDKSSGRSTLYIAASQPGDSATYLCAVRPTSGGSYIPTFGRGTSLIVHPPNIQNPDPAVY
QLRDSKSSDKSVCLFTDFDSQTNVSQSKDSDVYITDKCVLDMRSMDFKSNSAVAWSNKSDFAC
ANAFNNSIIPEDTFFPSPESSCDVKLVEKSFETDTNLNFQNLSVIGFRILLLKVAGFNLLMTLRLW
SS.
pHR_LaG17-TRBC1_TRAC_NoCysteineMod (P191), as depicted in FIG. 19 and having the
following translated amino acid sequence:
(SEQ ID NO: 104)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDFRKRRGKPIPNPLLGLDSTSGSGEGRGSLLTCGDVEENPGPMALPV
TALLLPLALLLHAARPYPYDVPDYAPNIQNPDPAVYQLRDSKSSDKSVCLFTDFDSQTNVSQSK
DSDVYITDKTVLDMRSMDFKSNSAVAWSNKSDFACANAFNNSIIPEDTFFPSPESSCDVKLVEKS
FETDTNLNFQNLSVIGFRILLLKVAGFNLLMTLRLWSS.
pHR_TRBC1_LaG17-TRAC_NoCysteineMod (P192), as depicted in FIG. 20 and having the
following translated amino acid sequence:
(SEQ ID NO: 105)
MALPVTALLLPLALLLHAARPYPYDVPDYAEDLNKVFPPEVAVFEPSEAEISHTQKATLVCLAT
GFFPDHVELSWWVNGKEVHSGVSTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPEQKLISEEDLMADVQLVESGGGLVQAGGSLRLSCAASGRTISMAAMSW
FRQAPGKEREFVAGISRSAGSAVHADSVKGRFTISRDNTKNTLYLQMNSLKAEDTAVYYCAVR
TSGFFGSIPRTGTAFDYWGQGTQVTVSPNIQNPDPAVYQLRDSKSSDKSVCLFTDFDSQTNVSQS
KDSDVYITDKTVLDMRSMDFKSNSAVAWSNKSDFACANAFNNSIIPEDTFFPSPESSCDVKLVE
KSFETDTNLNFQNLSVIGFRILLLKVAGFNLLMTLRLWSS.
pHR_LaG17-TRBC1_NoCysteineMod (P193), as depicted in FIG. 21 and having the following
translated amino acid sequence:
(SEQ ID NO: 106)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDF.
pHR_LaG17-TRAC_NoCysteineMod (P194), as depicted in FIG. 22 and having the following
translated amino acid sequence:
(SEQ ID NO: 107)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSPNIQNPDPAVYQLRDSKSSDKSVCLFTDFDSQ
TNVSQSKDSDVYITDKTVLDMRSMDFKSNSAVAWSNKSDFACANAFNNSIIPEDTFFPSPESSCD
VKLVEKSFETDTNLNFQNLSVIGFRILLLKVAGFNLLMTLRLWSS.
pHR_LaG17-TRBC1_IG4av-TRAC_NoCysteineMod (P195), as depicted in FIG. 23 and
having the following translated amino acid sequence:
(SEQ ID NO: 108)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDFRKRRGKPIPNPLLGLDSTSGSGEGRGSLLTCGDVEENPGPMALPV
TALLLPLALLLHAARPYPYDVPDYAMETLLGLLILWLQLQWVSSKQEVTQIPAALSVPEGENLV
LNCSFTDSAIYNLQWFRQDPGKGLTSLLLIQSSQREQTSGRLNASLDKSSGRSTLYIAASQPGDS
ATYLCAVRPTSGGSYIPTFGRGTSLIVHPPNIQNPDPAVYQLRDSKSSDKSVCLFTDFDSQTNVSQ
SKDSDVYITDKTVLDMRSMDFKSNSAVAWSNKSDFACANAFNNSIIPEDTFFPSPESSCDVKLVE
KSFETDTNLNFQNLSVIGFRILLLKVAGFNLLMTLRLWSS.
pHR_IG4bv-TRBC1_LaG17-TRAC_NoCysteineMod (P196), as depicted in FIG. 24 and
having the following translated amino acid sequence:
(SEQ ID NO: 109)
MALPVTALLLPLALLLHAARPYPYDVPDYAMSIGLLCCAALSLLWAGPVNAGVTQTPKFQVLK
TGQSMTLQCAQDMNHEYMSWYRQDPGMGLRLIHYSVGAGITDQGEVPNGYNVSRSTTEDFPL
RLLSAAPSQTSVYFCASSYVGNTGELFFGEGSRLTVLEDLNKVFPPEVAVFEPSEAEISHTQKATL
VCLATGFFPDHVELSWWVNGKEVHSGVSTDPQPLKEQPALNDSRYCLSSRLRVSATFWQNPRN
HFRCQVQFYGLSENDEWTQDRAKPVTQIVSAEAWGRADCGFTSVSYQQGVLSATILYEILLGK
ATLYAVLVSALVLMAMVKRKDFRKRRGKPIPNPLLGLDSTSGSGEGRGSLLTCGDVEENPGPM
ALPVTALLLPLALLLHAARPEQKLISEEDLMADVQLVESGGGLVQAGGSLRLSCAASGRTISMA
AMSWFRQAPGKEREFVAGISRSAGSAVHADSVKGRFTISRDNTKNTLYLQMNSLKAEDTAVYY
CAVRTSGFFGSIPRTGTAFDYWGQGTQVTVSPNIQNPDPAVYQLRDSKSSDKSVCLFTDFDSQT
NVSQSKDSDVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSS.
pHR_LaG17-TRBC1_TRAC_cp-TM_DomainSwap (P204), as depicted in FIG. 25 and having
the following translated amino acid sequence:
(SEQ ID NO: 110)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATNLSVIGFRILLLKV
AGFNLLMTLRLWSSRKRRGKPIPNPLLGLDSTSGSGEGRGSLLTCGDVEENPGPMALPVTALLL
PLALLLHAARPYPYDVPDYAPNIQNPDPAVYQLRDSKSSDKSVCLFTDFDSQTNVSQSKDSDVY
ITDKTVLDMRSMDFKSNSAVAWSNKSDFACANAFNNSIIPEDTFFPSPESSCDVKLVEKSFETDT
NLNFQILYEILLGKATLYAVLVSALVLMAMVKRKDF.
pHR_TRBC1_LaG17-TRAC_cp-TM_DomainSwap (P205), as depicted in FIG. 26 and having
the following translated amino acid sequence:
(SEQ ID NO: 111)
MALPVTALLLPLALLLHAARPYPYDVPDYAEDLNKVFPPEVAVFEPSEAEISHTQKATLVCLAT
GFFPDHVELSWWVNGKEVHSGVSTDPQPLKEQPALNDSRYCLSSRLRVSATFWQNPRNHFRCQ
VQFYGLSENDEWTQDRAKPVTQIVSAEAWGRADCGFTSVSYQQGVLSATNLSVIGFRILLLKVA
GFNLLMTLRLWSSRKRRGKPIPNPLLGLDSTSGSGEGRGSLLTCGDVEENPGPMALPVTALLLPL
ALLLHAARPEQKLISEEDLMADVQLVESGGGLVQAGGSLRLSCAASGRTISMAAMSWFRQAPG
KEREFVAGISRSAGSAVHADSVKGRFTISRDNTKNTLYLQMNSLKAEDTAVYYCAVRTSGFFGS
IPRTGTAFDYWGQGTQVTVSPNIQNPDPAVYQLRDSKSSDKSVCLFTDFDSQTNVSQSKDSDVY
ITDKTVLDMRSMDFKSNSAVAWSNKSDFACANAFNNSIIPEDTFFPSPESSCDVKLVEKSFETDT
NLNFQILYEILLGKATLYAVLVSALVLMAMVKRKDF.
pHR_LaG17-TRBC1_cp-TM_DomainSwap (P206), as depicted in FIG. 27 and having the
following translated amino acid sequence:
(SEQ ID NO: 112)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATNLSVIGFRILLLKV
AGFNLLMTLRLWSS.
pHR_LaG17-TRAC_cp-TM_DomainSwap (P207), as depicted in FIG. 28 and having the
following translated amino acid sequence:
(SEQ ID NO: 113)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSPNIQNPDPAVYQLRDSKSSDKSVCLFTDFDSQ
TNVSQSKDSDVYITDKTVLDMRSMDFKSNSAVAWSNKSDFACANAFNNSIIPEDTFFPSPESSCD
VKLVEKSFETDTNLNFQILYEILLGKATLYAVLVSALVLMAMVKRKDF.
pHR_LaG17-TRBC1_IG4av-TRAC_cp-TM_DomainSwap (P208), as depicted in FIG. 29 and
having the following translated amino acid sequence:
(SEQ ID NO: 114)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCGFTSVSYQQGVLSATNLSVIGFRILLLKV
AGFNLLMTLRLWSSRKRRGKPIPNPLLGLDSTSGSGEGRGSLLTCGDVEENPGPMALPVTALLL
PLALLLHAARPYPYDVPDYAMETLLGLLILWLQLQWVSSKQEVTQIPAALSVPEGENLVLNCSF
TDSAIYNLQWFRQDPGKGLTSLLLIQSSQREQTSGRLNASLDKSSGRSTLYIAASQPGDSATYLC
AVRPTSGGSYIPTFGRGTSLIVHPPNIQNPDPAVYQLRDSKSSDKSVCLFTDFDSQTNVSQSKDSD
VYITDKTVLDMRSMDFKSNSAVAWSNKSDFACANAFNNSIIPEDTFFPSPESSCDVKLVEKSFET
DTNLNFQILYEILLGKATLYAVLVSALVLMAMVKRKDF.
pHR_IG4bv-TRBC1_LaG17-TRAC_cp-TM_DomainSwap (P209), as depicted in FIG. 30 and
having the following translated amino acid sequence:
(SEQ ID NO: 115)
MALPVTALLLPLALLLHAARPYPYDVPDYAMSIGLLCCAALSLLWAGPVNAGVTQTPKFQVLK
TGQSMTLQCAQDMNHEYMSWYRQDPGMGLRLIHYSVGAGITDQGEVPNGYNVSRSTTEDFPL
RLLSAAPSQTSVYFCASSYVGNTGELFFGEGSRLTVLEDLNKVFPPEVAVFEPSEAEISHTQKATL
VCLATGFFPDHVELSWWVNGKEVHSGVSTDPQPLKEQPALNDSRYCLSSRLRVSATFWQNPRN
HFRCQVQFYGLSENDEWTQDRAKPVTQIVSAEAWGRADCGFTSVSYQQGVLSATNLSVIGFRIL
LLKVAGFNLLMTLRLWSSRKRRGKPIPNPLLGLDSTSGSGEGRGSLLTCGDVEENPGPMALPVT
ALLLPLALLLHAARPEQKLISEEDLMADVQLVESGGGLVQAGGSLRLSCAASGRTISMAAMSW
FRQAPGKEREFVAGISRSAGSAVHADSVKGRFTISRDNTKNTLYLQMNSLKAEDTAVYYCAVR
TSGFFGSIPRTGTAFDYWGQGTQVTVSPNIQNPDPAVYQLRDSKSSDKSVCLFTDFDSQTNVSQS
KDSDVYITDKTVLDMRSMDFKSNSAVAWSNKSDFACANAFNNSIIPEDTFFPSPESSCDVKLVE
KSFETDTNLNFQILYEILLGKATLYAVLVSALVLMAMVKRKDF.
pHR_LaG17-TRBC1_TRAC_C-cp_DomainSwap (P210), as depicted in FIG. 31 and having the
following translated amino acid sequence:
(SEQ ID NO: 116)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCDVKLVEKSFETDTNLNFQNLSVIGFRILL
LKVAGFNLLMTLRLWSSRKRRGKPIPNPLLGLDSTSGSGEGRGSLLTCGDVEENPGPMALPVTA
LLLPLALLLHAARPYPYDVPDYAPNIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCGFTSVSYQQG
VLSATILYEILLGKATLYAVLVSALVLMAMVKRKDF.
pHR_TRBC1_LaG17-TRAC_C-cp_DomainSwap (P211), as depicted in FIG. 32 and having the
following translated amino acid sequence:
(SEQ ID NO: 117)
MALPVTALLLPLALLLHAARPYPYDVPDYAEDLNKVFPPEVAVFEPSEAEISHTQKATLVCLAT
GFFPDHVELSWWVNGKEVHSGVSTDPQPLKEQPALNDSRYCLSSRLRVSATFWQNPRNHFRCQ
VQFYGLSENDEWTQDRAKPVTQIVSAEAWGRADCDVKLVEKSFETDTNLNFQNLSVIGFRILLL
KVAGFNLLMTLRLWSSRKRRGKPIPNPLLGLDSTSGSGEGRGSLLTCGDVEENPGPMALPVTAL
LLPLALLLHAARPEQKLISEEDLMADVQLVESGGGLVQAGGSLRLSCAASGRTISMAAMSWFR
QAPGKEREFVAGISRSAGSAVHADSVKGRFTISRDNTKNTLYLQMNSLKAEDTAVYYCAVRTS
GFFGSIPRTGTAFDYWGQGTQVTVSPNIQNPDPAVYQLRDSKSSDKSVCLFTDFDSQTNVSQSK
DSDVYITDKTVLDMRSMDFKSNSAVAWSNKSDFACANAFNNSIIPEDTFFPSPESSCGFTSVSYQ
QGVLSATILYEILLGKATLYAVLVSALVLMAMVKRKDF.
pHR_LaG17-TRBC1_C-cp_DomainSwap (P212), as depicted in FIG. 33 and having the
following translated amino acid sequence:
(SEQ ID NO: 118)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCDVKLVEKSFETDTNLNFQNLSVIGFRILL
LKVAGFNLLMTLRLWSS.
pHR_LaG17-TRAC_C-cp_DomainSwap (P213), as depicted in FIG. 34 and having the following
translated amino acid sequence:
(SEQ ID NO: 119)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSPNIQNPDPAVYQLRDSKSSDKSVCLFTDFDSQ
TNVSQSKDSDVYITDKTVLDMRSMDFKSNSAVAWSNKSDFACANAFNNSIIPEDTFFPSPESSCG
FTSVSYQQGVLSATILYEILLGKATLYAVLVSALVLMAMVKRKDF.
pHR_LaG17-TRBC1_IG4av-TRAC_C-cp_DomainSwap (P214), as depicted in FIG. 35 and having
the following translated amino acid sequence:
(SEQ ID NO: 120)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVSTDPQPLKEQPALNDSRYCLSSRLRVSATFWQNPRNHFRC
QVQFYGLSENDEWTQDRAKPVTQIVSAEAWGRADCDVKLVEKSFETDTNLNFQNLSVIGFRILL
LKVAGFNLLMTLRLWSSRKRRGKPIPNPLLGLDSTSGSGEGRGSLLTCGDVEENPGPMALPVTA
LLLPLALLLHAARPYPYDVPDYAMETLLGLLILWLQLQWVSSKQEVTQIPAALSVPEGENLVLN
CSFTDSAIYNLQWFRQDPGKGLTSLLLIQSSQREQTSGRLNASLDKSSGRSTLYIAASQPGDSAT
YLCAVRPTSGGSYIPTFGRGTSLIVHPPNIQNPDPAVYQLRDSKSSDKSVCLFTDFDSQTNVSQSK
DSDVYITDKTVLDMRSMDFKSNSAVAWSNKSDFACANAFNNSIIPEDTFFPSPESSCGFTSVSYQ
QGVLSATILYEILLGKATLYAVLVSALVLMAMVKRKDF.
pHR_IG4bv-TRBC1_LaG17-TRAC_C-cp_DomainSwap (P215), as depicted in FIG. 36 and having
the following translated amino acid sequence:
(SEQ ID NO: 121)
MALPVTALLLPLALLLHAARPYPYDVPDYAMSIGLLCCAALSLLWAGPVNAGVTQTPKFQVLK
TGQSMTLQCAQDMNHEYMSWYRQDPGMGLRLIHYSVGAGITDQGEVPNGYNVSRSTTEDFPL
RLLSAAPSQTSVYFCASSYVGNTGELFFGEGSRLTVLEDLNKVFPPEVAVFEPSEAEISHTQKATL
VCLATGFFPDHVELSWWVNGKEVHSGVSTDPQPLKEQPALNDSRYCLSSRLRVSATFWQNPRN
HFRCQVQFYGLSENDEWTQDRAKPVTQIVSAEAWGRADCDVKLVEKSFETDTNLNFQNLSVIG
FRILLLKVAGFNLLMTLRLWSSRKRRGKPIPNPLLGLDSTSGSGEGRGSLLTCGDVEENPGPMAL
PVTALLLPLALLLHAARPEQKLISEEDLMADVQLVESGGGLVQAGGSLRLSCAASGRTISMAAM
SWFRQAPGKEREFVAGISRSAGSAVHADSVKGRFTISRDNTKNTLYLQMNSLKAEDTAVYYCA
VRTSGFFGSIPRTGTAFDYWGQGTQVTVSPNIQNPDPAVYQLRDSKSSDKSVCLFTDFDSQTNVS
QSKDSDVYITDKTVLDMRSMDFKSNSAVAWSNKSDFACANAFNNSIIPEDTFFPSPESSCGFTSV
SYQQGVLSATILYEILLGKATLYAVLVSALVLMAMVKRKDF.
pHR_LaG17-TRBC1_aMeso-TRAC (P254), as depicted in FIG. 37 and having the following
translated amino acid sequence:
(SEQ ID NO: 122)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSEDLNKVFPPEVAVFEPSEAEISHTQKATLVCLA
TGFFPDHVELSWWVNGKEVHSGVCTDPQPLKEQPALNDSRYCLSSRLRVSATFWQNPRNHFRC
QVQFYGLSENDEWTQDRAKPVTQIVSAEAWGRADCGFTSVSYQQGVLSATILYEILLGKATLY
AVLVSALVLMAMVKRKDFRKRRGKPIPNPLLGLDSTSGSGEGRGSLLTCGDVEENPGPMALPV
TALLLPLALLLHAARPDYKDDDDKGSQVQLQQSGPELEKPGASVKISCKASGYSFTGYTMNWV
KQSHGKSLEWIGLITPYNGASSYNQKFRGKATLTVDKSSSTAYMDLLSLTSEDSAVYFCARGGY
DGRGFDYWGQGTTVTVSSGGGGSGGGGSSGGGSDIELTQSPAIMSASPGEKVTMTCSASSSVSY
MHWYQQKSGTSPKRWIYDTSKLASGVPGRFSGSGSGNSYSLTISSVEAEDDATYYCQQWSKHP
LTYGAGTKLEIKASPNIQNPDPAVYQLRDSKSSDKSVCLFTDFDSQTNVSQSKDSDVYITDKCVL
DMRSMDFKSNSAVAWSNKSDFACANAFNNSIIPEDTFFPSPESSCDVKLVEKSFETDTNLNFQNL
SVIGFRILLLKVAGFNLLMTLRLWSS.
pHR_aMeso-TRBC1_LaG17-TRAC (P255), as depicted in FIG. 38 and having the following
translated amino acid sequence:
(SEQ ID NO: 123)
MALPVTALLLPLALLLHAARPDYKDDDDKGSQVQLQQSGPELEKPGASVKISCKASGYSFTGY
TMNWVKQSHGKSLEWIGLITPYNGASSYNQKFRGKATLTVDKSSSTAYMDLLSLTSEDSAVYF
CARGGYDGRGFDYWGQGTTVTVSSGGGGSGGGGSSGGGSDIELTQSPAIMSASPGEKVTMTCS
ASSSVSYMHWYQQKSGTSPKRWIYDTSKLASGVPGRFSGSGSGNSYSLTISSVEAEDDATYYCQ
QWSKHPLTYGAGTKLEIKASEDLNKVFPPEVAVFEPSEAEISHTQKATLVCLATGFFPDHVELS
WWVNGKEVHSGVCTDPQPLKEQPALNDSRYCLSSRLRVSATFWQNPRNHFRCQVQFYGLSEN
DEWTQDRAKPVTQIVSAEAWGRADCGFTSVSYQQGVLSATILYEILLGKATLYAVLVSALVLM
AMVKRKDFRKRRGKPIPNPLLGLDSTSGSGEGRGSLLTCGDVEENPGPMALPVTALLLPLALLL
HAARPEQKLISEEDLMADVQLVESGGGLVQAGGSLRLSCAASGRTISMAAMSWFRQAPGKERE
FVAGISRSAGSAVHADSVKGRFTISRDNTKNTLYLQMNSLKAEDTAVYYCAVRTSGFFGSIPRT
GTAFDYWGQGTQVTVSPNIQNPDPAVYQLRDSKSSDKSVCLFTDFDSQTNVSQSKDSDVYITDK
CVLDMRSMDFKSNSAVAWSNKSDFACANAFNNSIIPEDTFFPSPESSCDVKLVEKSFETDTNLNF
QNLSVIGFRILLLKVAGFNLLMTLRLWSS.
pHR_aMeso_LaG17-TRBC1-TRAC (P256), as depicted in FIG. 39 and having the following
translated amino acid sequence:
(SEQ ID NO: 124)
MALPVTALLLPLALLLHAARPDYKDDDDKGSQVQLQQSGPELEKPGASVKISCKASGYSFTGY
TMNWVKQSHGKSLEWIGLITPYNGASSYNQKFRGKATLTVDKSSSTAYMDLLSLTSEDSAVYF
CARGGYDGRGFDYWGQGTTVTVSSGGGGSGGGGSSGGGSDIELTQSPAIMSASPGEKVTMTCS
ASSSVSYMHWYQQKSGTSPKRWIYDTSKLASGVPGRFSGSGSGNSYSLTISSVEAEDDATYYCQ
QWSKHPLTYGAGTKLEIKASGGGGSGGGGSGGGGSMADVQLVESGGGLVQAGGSLRLSCAAS
GRTISMAAMSWFRQAPGKEREFVAGISRSAGSAVHADSVKGRFTISRDNTKNTLYLQMNSLKA
EDTAVYYCAVRTSGFFGSIPRTGTAFDYWGQGTQVTVSEDLNKVFPPEVAVFEPSEAEISHTQK
ATLVCLATGFFPDHVELSWWVNGKEVHSGVCTDPQPLKEQPALNDSRYCLSSRLRVSATFWQN
PRNHFRCQVQFYGLSENDEWTQDRAKPVTQIVSAEAWGRADCGFTSVSYQQGVLSATILYEILL
GKATLYAVLVSALVLMAMVKRKDFRKRRGKPIPNPLLGLDSTSGSGEGRGSLLTCGDVEENPG
PMALPVTALLLPLALLLHAARPYPYDVPDYAPNIQNPDPAVYQLRDSKSSDKSVCLFTDFDSQT
NVSQSKDSDVYITDKCVLDMRSMDFKSNSAVAWSNKSDFACANAFNNSIIPEDTFFPSPESSCD
VKLVEKSFETDTNLNFQNLSVIGFRILLLKVAGFNLLMTLRLWSS.
pHR_TRBC1_aMeso_LaG17-TRAC (P257), as depicted in FIG. 40 and having the following
translated amino acid sequence:
(SEQ ID NO: 125)
MALPVTALLLPLALLLHAARPYPYDVPDYAEDLNKVFPPEVAVFEPSEAEISHTQKATLVCLAT
GFFPDHVELSWWVNGKEVHSGVCTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPDYKDDDDKGSQVQLQQSGPELEKPGASVKISCKASGYSFTGYTMNWVK
QSHGKSLEWIGLITPYNGASSYNQKFRGKATLTVDKSSSTAYMDLLSLTSEDSAVYFCARGGYD
GRGFDYWGQGTTVTVSSGGGGSGGGGSSGGGSDIELTQSPAIMSASPGEKVTMTCSASSSVSY
MHWYQQKSGTSPKRWIYDTSKLASGVPGRFSGSGSGNSYSLTISSVEAEDDATYYCQQWSKHP
LTYGAGTKLEIKASGGGGSGGGGSGGGGSMADVQLVESGGGLVQAGGSLRLSCAASGRTISMA
AMSWFRQAPGKEREFVAGISRSAGSAVHADSVKGRFTISRDNTKNTLYLQMNSLKAEDTAVYY
CAVRTSGFFGSIPRTGTAFDYWGQGTQVTVSPNIQNPDPAVYQLRDSKSSDKSVCLFTDFDSQT
NVSQSKDSDVYITDKCVLDMRSMDFKSNSAVAWSNKSDFACANAFNNSIIPEDTFFPSPESSCD
VKLVEKSFETDTNLNFQNLSVIGFRILLLKVAGFNLLMTLRLWSS.
pHR_LaG17_aMeso-TRBC1_TRAC (P258), as depicted in FIG. 41 and having the following
translated amino acid sequence:
(SEQ ID NO: 126)
MALPVTALLLPLALLLHAARPEQKLISEEDLMADVQLVESGGGLVQAGGSLRLSCAASGRTISM
AAMSWFRQAPGKEREFVAGISRSAGSAVHADSVKGRFTISRDNTKNTLYLQMNSLKAEDTAVY
YCAVRTSGFFGSIPRTGTAFDYWGQGTQVTVSGGGGSGGGGSGGGGSSGPELEKPGASVKISCK
ASGYSFTGYTMNWVKQSHGKSLEWIGLITPYNGASSYNQKFRGKATLTVDKSSSTAYMDLLSL
TSEDSAVYFCARGGYDGRGFDYWGQGTTVTVSSGGGGSGGGGSSGGGSDIELTQSPAIMSASP
GEKVTMTCSASSSVSYMHWYQQKSGTSPKRWIYDTSKLASGVPGRFSGSGSGNSYSLTISSVEA
EDDATYYCQQWSKHPLTYGAGTKLEIKASEDLNKVFPPEVAVFEPSEAEISHTQKATLVCLATG
FFPDHVELSWWVNGKEVHSGVCTDPQPLKEQPALNDSRYCLSSRLRVSATFWQNPRNHFRCQV
QFYGLSENDEWTQDRAKPVTQIVSAEAWGRADCGFTSVSYQQGVLSATILYEILLGKATLYAV
LVSALVLMAMVKRKDFRKRRGKPIPNPLLGLDSTSGSGEGRGSLLTCGDVEENPGPMALPVTA
LLLPLALLLHAARPYPYDVPDYAPNIQNPDPAVYQLRDSKSSDKSVCLFTDFDSQTNVSQSKDS
DVYITDKCVLDMRSMDFKSNSAVAWSNKSDFACANAFNNSIIPEDTFFPSPESSCDVKLVEKSFE
TDTNLNFQNLSVIGFRILLLKVAGFNLLMTLRLWSS.
pHR_TRBC1_LaG17_aMeso-TRAC (P259), as depicted in FIG. 42 and having the following
translated amino acid sequence:
(SEQ ID NO: 127)
MALPVTALLLPLALLLHAARPYPYDVPDYAEDLNKVFPPEVAVFEPSEAEISHTQKATLVCLAT
GFFPDHVELSWWVNGKEVHSGVCTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPEQKLISEEDLMADVQLVESGGGLVQAGGSLRLSCAASGRTISMAAMSW
FRQAPGKEREFVAGISRSAGSAVHADSVKGRFTISRDNTKNTLYLQMNSLKAEDTAVYYCAVR
TSGFFGSIPRTGTAFDYWGQGTQVTVSGGGGSGGGGSGGGGSSGPELEKPGASVKISCKASGYS
FTGYTMNWVKQSHGKSLEWIGLITPYNGASSYNQKFRGKATLTVDKSSSTAYMDLLSLTSEDS
AVYFCARGGYDGRGFDYWGQGTTVTVSSGGGGSGGGGSSGGGSDIELTQSPAIMSASPGEKVT
MTCSASSSVSYMHWYQQKSGTSPKRWIYDTSKLASGVPGRFSGSGSGNSYSLTISSVEAEDDAT
YYCQQWSKHPLTYGAGTKLEIKASPNIQNPDPAVYQLRDSKSSDKSVCLFTDFDSQTNVSQSKD
SDVYITDKCVLDMRSMDFKSNSAVAWSNKSDFACANAFNNSIIPEDTFFPSPESSCDVKLVEKSF
ETDTNLNFQNLSVIGFRILLLKVAGFNLLMTLRLWSS.

Chimeric TCRs having paired modified alpha and beta chains were expressed in human CD8(+) T cells and T cell activation (CD69 expression) and target cell killing were assessed relative to controls. T cell activation and killing of target cells expressing the relevant antigen (GFP) as a result of the expressed chimeric TCR was evaluated in comparison to untransduced T cells (“untransduced”, negative control) and T cells transduced with an anti-GFP chimeric antigen receptor (α-GFP CAR, “P29”; positive control). Results are provided for an anti-GFP chimeric TCR having an anti-GFP nanobody (LaG17) fused to a truncated TCR alpha chain paired with a truncated TCR beta chain (P148, described above), where both chains include corresponding cysteine modifications resulting in a recombinant disulfide bond between the two chains.

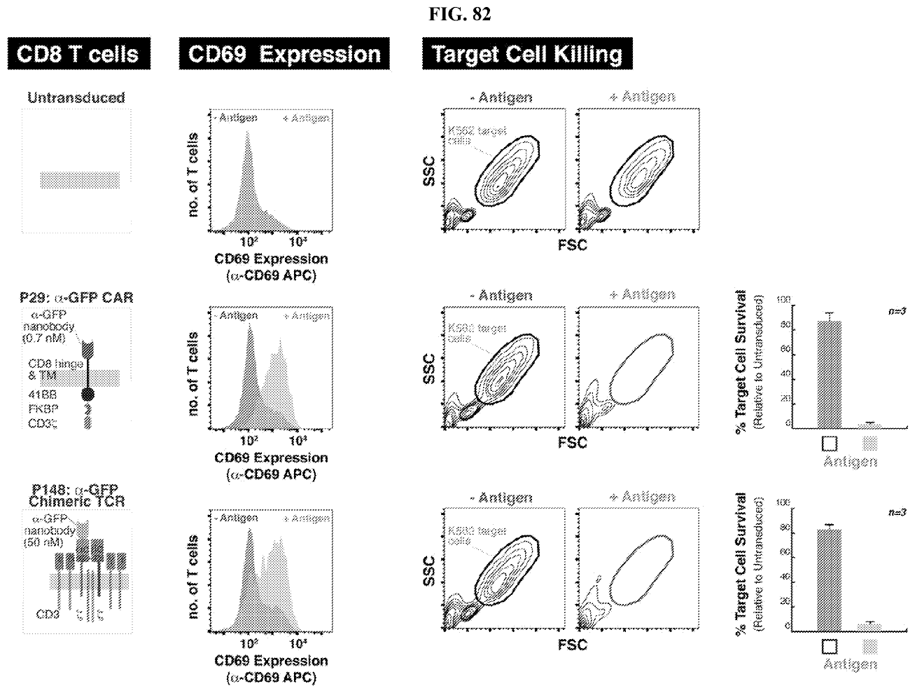

As can be seen in FIG. 82, untransduced negative control T cells were not activated (CD69 expression) in the presence of the relevant antigen (GFP, “+Antigen”) and such cells did not show antigen specific target cell killing (top panel). Transduction of T cells with the anti-GPF chimeric TCR resulted in antigen specific T cell activation as measured by CD69 expression as well as specific killing of antigen (GFP, “+Antigen”) expressing K562 target cells (bottom panel). This antigen specific T cell activation and target cell killing was comparable to that seen in T cell transduced with the anti-GFP CAR positive control (middle panel).

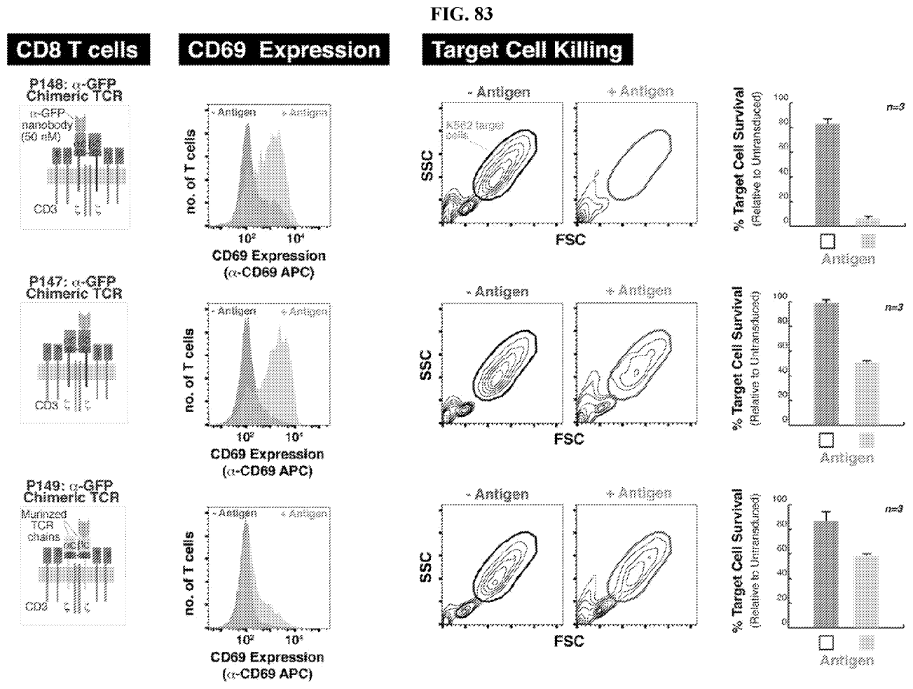

CD8(+) T cell activation and antigen specific target cell killing was assessed using various chimeric TCR constructs, including where the antigen binding domain was fused to either the alpha chain or the beta chain. FIG. 83 shows T cell activation (CD69 expression) and antigen specific target cell killing resulting from transduction of human CD8(+) T cells with constructs P148, P147 and P149 (described above).

Constructs were tested for immune cell activation in contexts other than human CD8(+) T cells. For example, as shown in FIG. 84, Jurkat T cells transduced with anti-GFP chimeric TCR (P147) showed antigen specific immune cell activation (CD69 expression; “Antigen+”). Such activation is in comparison to the lack of activation seen when the P147 transduced cells were assayed in the absence of antigen (“−Antigen”). FIG. 84 also provides for comparison the levels of CD69 expression in untransduced negative control Jurkat T cells and anti-GPF CAR transduced positive control cells.

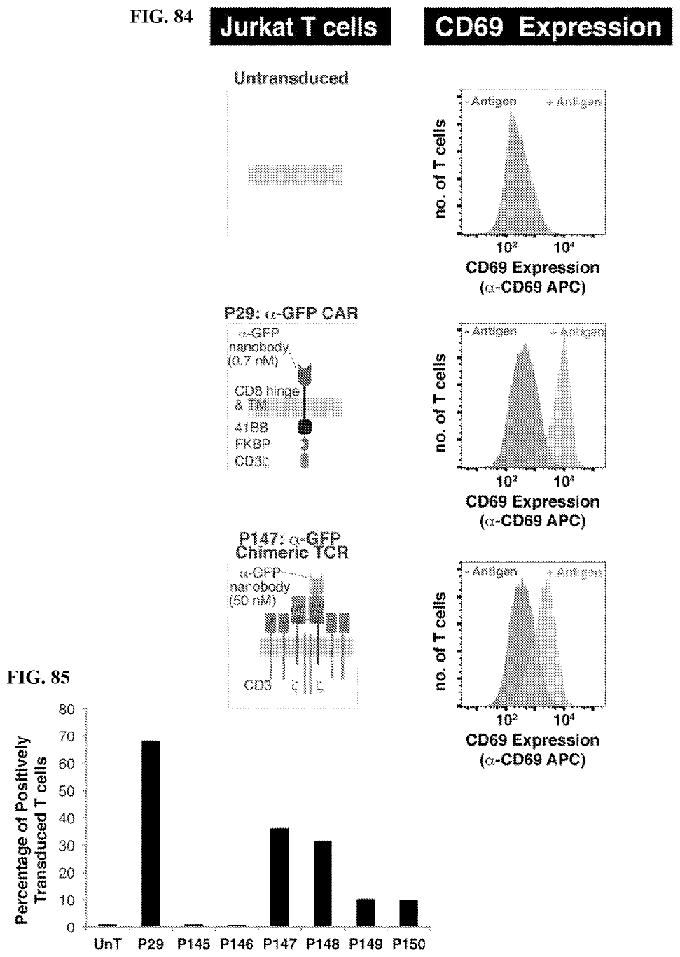

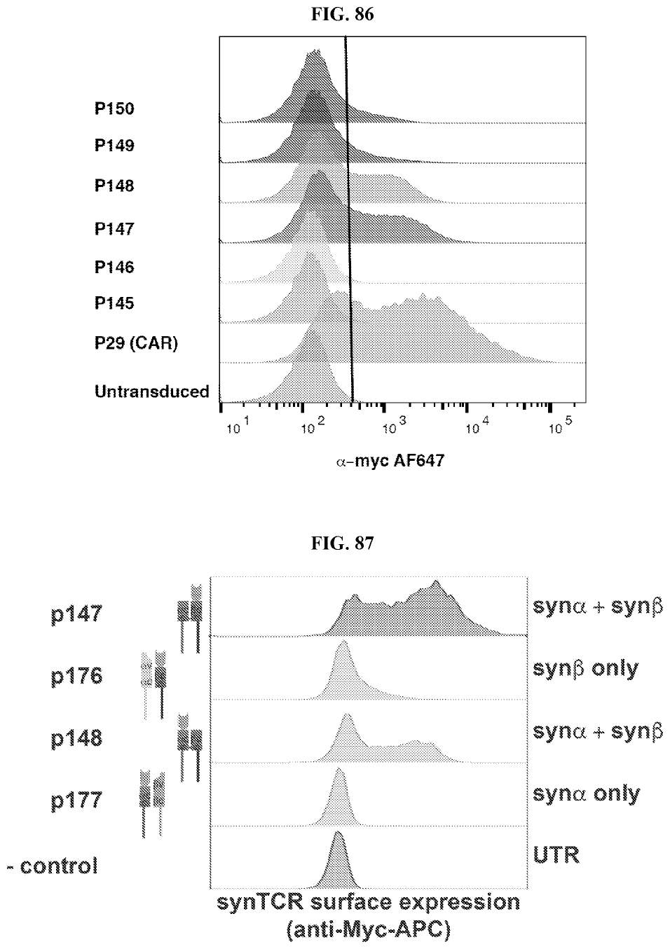

The expression of various chimeric TCR constructs was further investigated. FIG. 85, provides quantification of the percent positively transduced T cells for constructs P145-P150 as well as negative (untransduced, “UnT”) and positive (P29) controls. Cell surface expression of transduced chimeric TCRs (P145-P150), as well as negative (“untransduced”) and positive (P29) controls, was evaluated using a fluorescent anti-myc antibody (“a-myc AF647”) (FIG. 86).

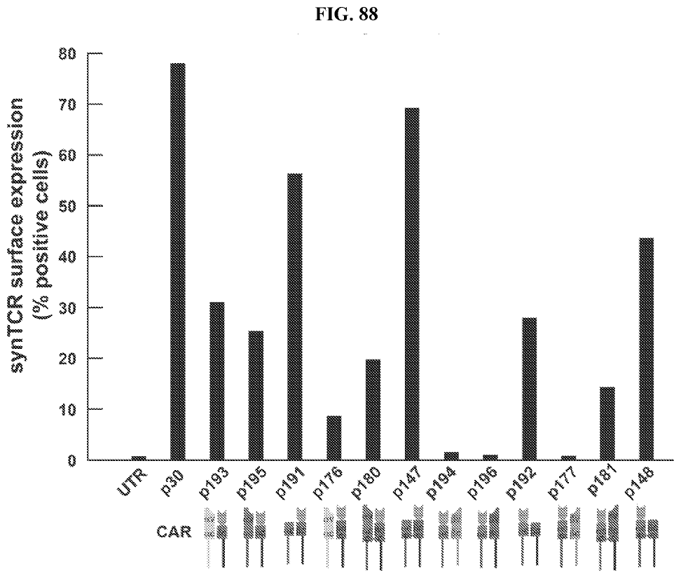

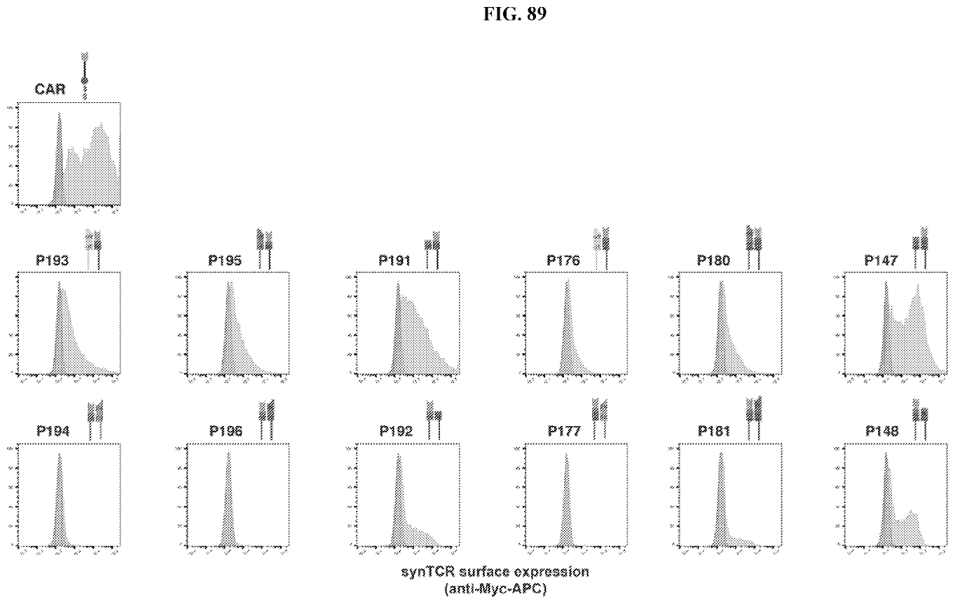

In this example, paired expression of a modified TCR alpha chain along with a modified TCR beta chain resulted in superior cell surface expression as compared to the expression of single chains (i.e., chains not paired with a corresponding engineered chain). Individually expressed chains rely on pairing with endogenously expressed chains to form a TCR complex. For example, an individually expressed modified alpha chain, having a fused antigen binding domain, would rely on pairing with an endogenous beta chain to form a TCR and an individually expressed modified beta chain, e.g., having a fused antigen binding domain, would rely on pairing with an endogenous alpha chain to form a TCR. As can be seen in the example of FIG. 87, cell surface expression of paired modified (or “synthetic”) alpha and beta truncated chains having a recombinant disulfide bond (“synα+synβ”) was superior to cell surface expression of the individual synthetic chains (“synβ only” or “synα only”) regardless of whether the antigen binding domain was fused to the alpha or beta chain (compare synTCR surface expression (as measured by anit-Myc-APC) of p147 vs. P176 and p148 vs. p177). FIG. 88 provides quantification of synTCR cell surface expression for various constructs described herein and FIG. 89 provides the corresponding FACS profiles.

The above examples demonstrate that chimeric TCRs having modified/synthetic alpha and/or beta chains can be effectively expressed on the surface of immune cells allowing TCR-based antigen specific immune cell activation and/or target cell killing to be redirected to an antigen of choice. These results further support the increased cell surface expression of paired modified alpha and beta chains as compared to modified chains expressed individually, thus supporting the use of chimeric TCRs having paired modified/synthetic alpha and beta chains. These chimeric TCRs or “synTCRs” combine the signaling capability of the TCR complex with the modular recognition domain targeting ability afforded by CARs.

### Example 2: Comparative Efficacy of synTCR and CAR Directed to Surface Antigen

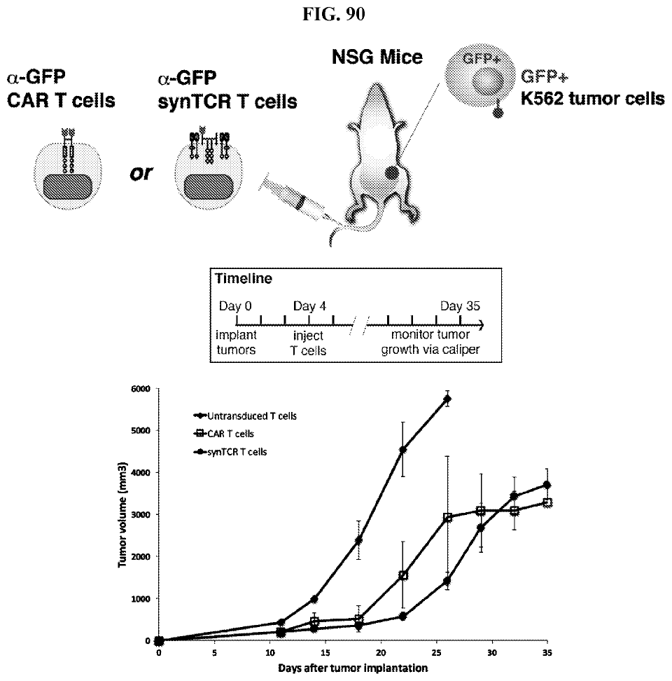

To compare the efficacy of a synTCR and a CAR, both targeting surface-expressed GFP as antigen, a xenograft tumor experiment was performed. Specifically, NSG mice were implanted with 5×10⁶ GFP+ K562 target cells in the right flank. After tumor engraftment for 4 days, 4×10⁶ each primary human CD4 and CD8 T cells were injected i.v. in the tail vein of the mice. The T cell groups were: (1) untransduced T cells (“Untransduced”), (2) T cells transduced with CAR targeting GFP (“anti-GFP CAR”), and (3) T cells transduced with synTCR targeting GFP (“anti-GFP synTCR”). The synTCR employed in this experiment was an alpha-fusion. Tumors grew rapidly in the mice in the control group (i.e., “Untransduced”). Mice in both of the treatment groups, i.e., the anti-GFP CAR and anti-GFP synTCR groups, displayed significantly delayed tumor growth. No significant differences were seen in tumor management by CAR vs synTCR T cells (FIG. 90).

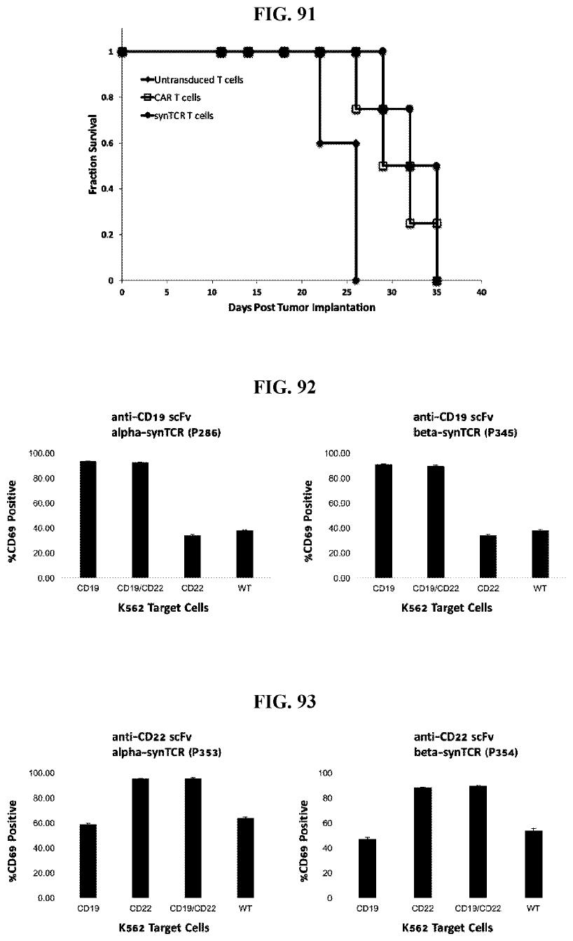

The CAR and synTCR T cells had similar effects on delaying time to euthanasia relative to untransduced T cells (FIG. 91). Collectively, these results demonstrate, in an NSG mouse xenograft solid tumor model, that synTCR T cells inhibit tumor growth and extend survival at least as well as CAR T cells targeting the same antigen.

### Example 3: synTCR with a scFv Antigen-Binding Domain

To demonstrate the modularity of the synTCR receptor platform, scFvs were introduced as the antigen-binding domain in further constructs and these constructs were subsequently tested for antigen-specific immune activation. Specifically, primary human CD8 T cells were transduced with two different anti-CD19 synTCRs: “alpha-synTCR” (P286, anti-CD19 scFv fused to truncated TCR alpha chain paired with truncated beta chain) and “beta-synTCR” (P345, anti-CD19 scFv fused to truncated TCR beta chain paired with truncated alpha chain). For both anti-CD19 synTCRs, both truncated TCR chains include corresponding cysteine modifications resulting in a recombinant disulfide bond between the two chains. SynTCR-expressing CD8 T cells were co-cultured overnight with K562 target cells expressing different antigens (i.e., exogenous CD19, exogenous CD22, exogenous CD19 and CD22 (“CD19/CD22”) or no exogenous antigen (“WT”)). After 24 hours of co-culture, T cell activation was assayed flow cytometrically by quantifying the level of CD69 expression. For both anti-CD19 alpha-synTCR and beta-synTCR T cells, CD69 expression was upregulated in the presence of CD19+ target cells relative to CD19− target cells (FIG. 92).

To further demonstrate the versatility of the scFv targeting approach in synTCRs, primary human CD8 T cells were transduced with anti-CD22 scFv antigen-binding domain containing alpha-synTCR (P353) and beta-synTCR (P354). SynTCR-expressing CD8 T cells were co-cultured overnight with K562 target cells expressing different antigens (i.e., exogenous CD19, exogenous CD22, both exogenous CD19 and CD22 (“CD19/CD22”) or no exogenous antigen (“WT”)). After 24 hours of co-culture, T cell activation was assayed flow cytometrically by quantifying the level of CD69 expression. For both anti-CD22 alpha-synTCR and anti-CD22 beta-synTCR T cells, CD69 expression was upregulated in the presence of CD22+ target cells relative to CD22− target cells (FIG. 93).

Antigen-specific T cell activation driven by anti-CD19 and anti-CD22 synTCRs demonstrates that various antigen-binding domains may be employed on the synTCR platform, including various different scFvs targeting different antigens, providing wide versatility in antigen-specific targeting.

The above described constructs include the following:

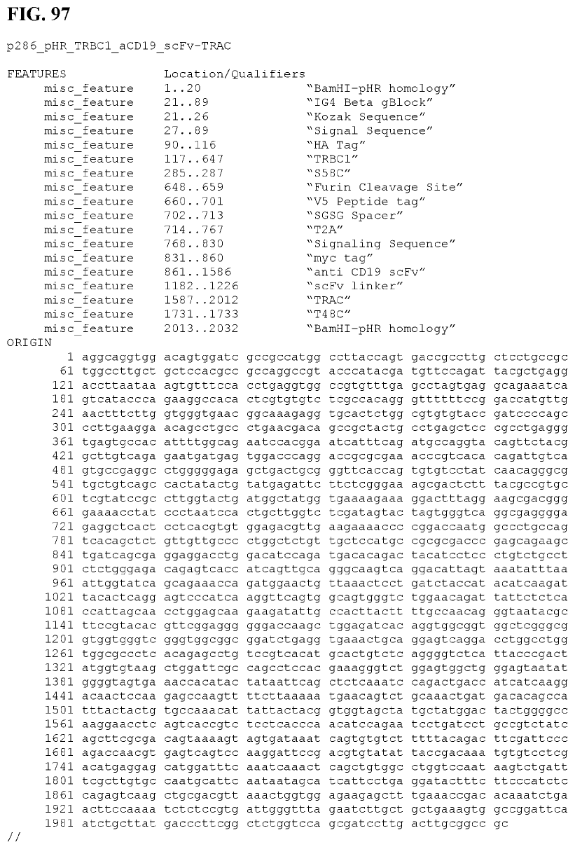

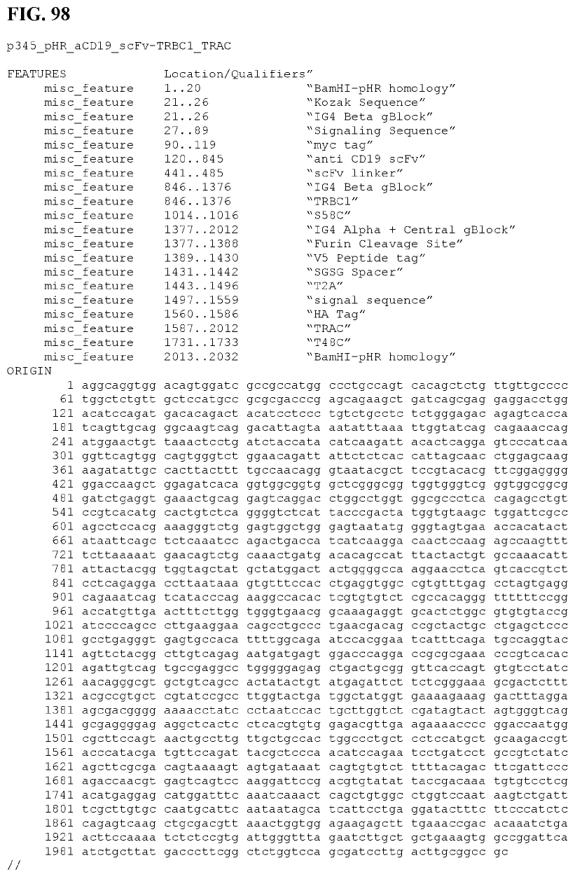

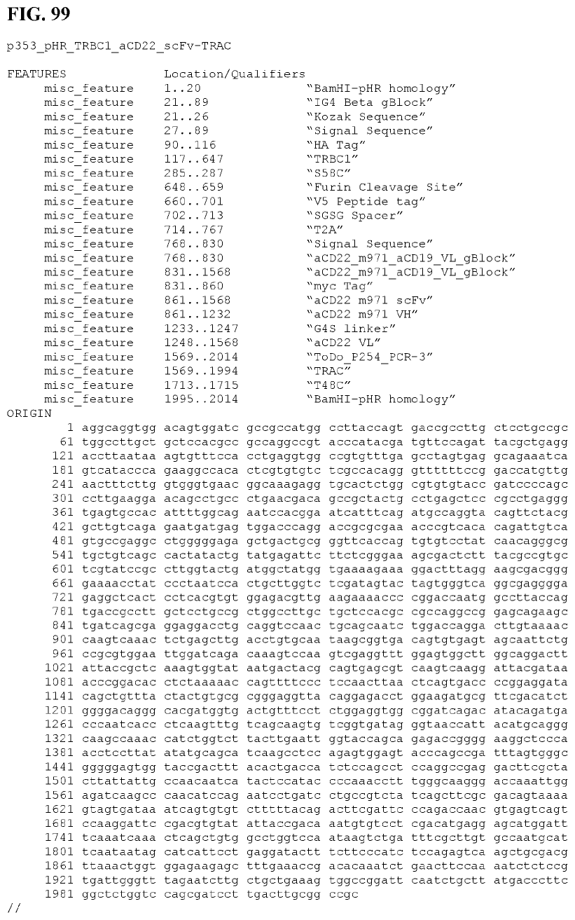

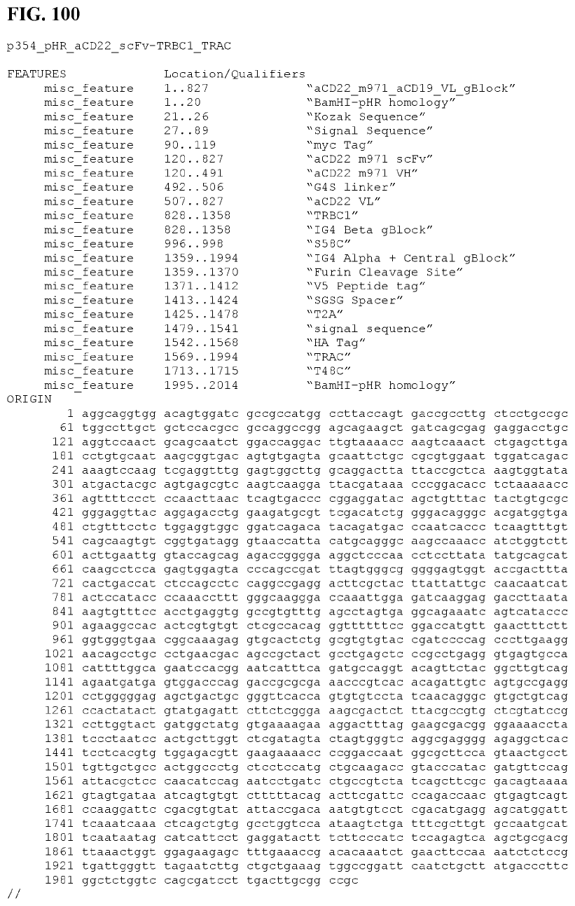

pHR_TRBC1_aCD19_scFv-TRAC (P286), as depicted in FIG. 97 and having the following
translated amino acid sequence:
(SEQ ID NO: 128)
MALPVTALLLPLALLLHAARPYPYDVPDYAEDLNKVFPPEVAVFEPSEAEISHTQKATLVCLAT
GFFPDHVELSWWVNGKEVHSGVCTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPEQKLISEEDLDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTKLEI
TGGGGSGGGGSGGGGSEVKLQESGPGLVAPSQSLSVTCTVSGVSLPDYGVSWIRQPPRKGLEW
LGVIWGSETTYYNSALKSRLTIIKDNSKSQVFLKMNSLQTDDTAIYYCAKHYYYGGSYAMDYW
GQGTSVTVSSPNIQNPDPAVYQLRDSKSSDKSVCLFTDFDSQTNVSQSKDSDVYITDKCVLDMR
SMDFKSNSAVAWSNKSDFACANAFNNSIIPEDTFFPSPESSCDVKLVEKSFETDTNLNFQNLSVIG
FRILLLKVAGFNLLMTLRLWSSDP.
pHR_aCD19_scFv-TRBC1_TRAC (P345), as depicted in FIG. 98 and having the following
translated amino acid sequence:
(SEQ ID NO: 129)
MALPVTALLLPLALLLHAARPEQKLISEEDLDIQMTQTTSSLSASLGDRVTISCRASQDISKYLN
WYQQKPDGTVKLLIYHTSRLHSGVPSRFSGSGSGTDYSLTISNLEQEDIATYFCQQGNTLPYTFG
GGTKLEITGGGGSGGGGSGGGGSEVKLQESGPGLVAPSQSLSVTCTVSGVSLPDYGVSWIRQPP
RKGLEWLGVIWGSETTYYNSALKSRLTIIKDNSKSQVFLKMNSLQTDDTAIYYCAKHYYYGGS
YAMDYWGQGTSVTVSSEDLNKVFPPEVAVFEPSEAEISHTQKATLVCLATGFFPDHVELSWWV
NGKEVHSGVCTDPQPLKEQPALNDSRYCLSSRLRVSATFWQNPRNHFRCQVQFYGLSENDEWT
QDRAKPVTQIVSAEAWGRADCGFTSVSYQQGVLSATILYEILLGKATLYAVLVSALVLMAMVK
RKDFRKRRGKPIPNPLLGLDSTSGSGEGRGSLLTCGDVEENPGPMALPVTALLLPLALLLHAARP
YPYDVPDYAPNIQNPDPAVYQLRDSKSSDKSVCLFTDFDSQTNVSQSKDSDVYITDKCVLDMRS
MDFKSNSAVAWSNKSDFACANAFNNSIIPEDTFFPSPESSCDVKLVEKSFETDTNLNFQNLSVIGF
RILLLKVAGFNLLMTLRLWSSDP.
pHR_TRBC1_aCD22_scFv-TRAC (P353), as depicted in FIG. 99 and having the following
translated amino acid sequence:
(SEQ ID NO: 130)
MALPVTALLLPLALLLHAARPYPYDVPDYAEDLNKVFPPEVAVFEPSEAEISHTQKATLVCLAT
GFFPDHVELSWWVNGKEVHSGVCTDPQPLKEQPALNDSRYCLSSRLRVSATFWQNPRNHFRCQ
VQFYGLSENDEWTQDRAKPVTQIVSAEAWGRADCGFTSVSYQQGVLSATILYEILLGKATLYA
VLVSALVLMAMVKRKDFRKRRGKPIPNPLLGLDSTSGSGEGRGSLLTCGDVEENPGPMALPVT
ALLLPLALLLHAARPEQKLISEEDLQVQLQQSGPGLVKPSQTLSLTCAISGDSVSSNSAAWNWIR
QSPSRGLEWLGRTYYRSKWYNDYAVSVKSRITINPDTSKNQFSLQLNSVTPEDTAVYYCAREVT
GDLEDAFDIWGQGTMVTVSSGGGGSDIQMTQSPSSLSASVGDRVTITCRASQTIWSYLNWYQQ
RPGKAPNLLIYAASSLQSGVPSRFSGRGSGTDFTLTISSLQAEDFATYYCQQSYSIPQTFGQGTKL
EIKPNIQNPDPAVYQLRDSKSSDKSVCLFTDFDSQTNVSQSKDSDVYITDKCVLDMRSMDFKSN
SAVAWSNKSDFACANAFNNSIIPEDTFFPSPESSCDVKLVEKSFETDTNLNFQNLSVIGFRILLLK
VAGFNLLMTLRLWSSDP.
pHR_aCD22_scFv-TRBC1_TRAC (P354), as depicted in FIG. 100 and having the following
translated amino acid sequence:
(SEQ ID NO: 131)
MALPVTALLLPLALLLHAARPEQKLISEEDLQVQLQQSGPGLVKPSQTLSLTCAISGDSVSSNSA
AWNWIRQSPSRGLEWLGRTYYRSKWYNDYAVSVKSRITINPDTSKNQFSLQLNSVTPEDTAVY
YCAREVTGDLEDAFDIWGQGTMVTVSSGGGGSDIQMTQSPSSLSASVGDRVTITCRASQTIWSY
LNWYQQRPGKAPNLLIYAASSLQSGVPSRFSGRGSGTDFTLTISSLQAEDFATYYCQQSYSIPQTF
GQGTKLEIKEDLNKVFPPEVAVFEPSEAEISHTQKATLVCLATGFFPDHVELSWWVNGKEVHSG
VCTDPQPLKEQPALNDSRYCLSSRLRVSATFWQNPRNHFRCQVQFYGLSENDEWTQDRAKPVT
QIVSAEAWGRADCGFTSVSYQQGVLSATILYEILLGKATLYAVLVSALVLMAMVKRKDFRKRR
GKPIPNPLLGLDSTSGSGEGRGSLLTCGDVEENPGPMALPVTALLLPLALLLHAARPYPYDVPDY
APNIQNPDPAVYQLRDSKSSDKSVCLFTDFDSQTNVSQSKDSDVYITDKCVLDMRSMDFKSNSA
VAWSNKSDFACANAFNNSIIPEDTFFPSPESSCDVKLVEKSFETDTNLNFQNLSVIGFRILLLKVA
GFNLLMTLRLWSSDP.

### Example 4: Dual-Antigen Binding Domain Containing synTCRs (“Dual-synTCRs”)

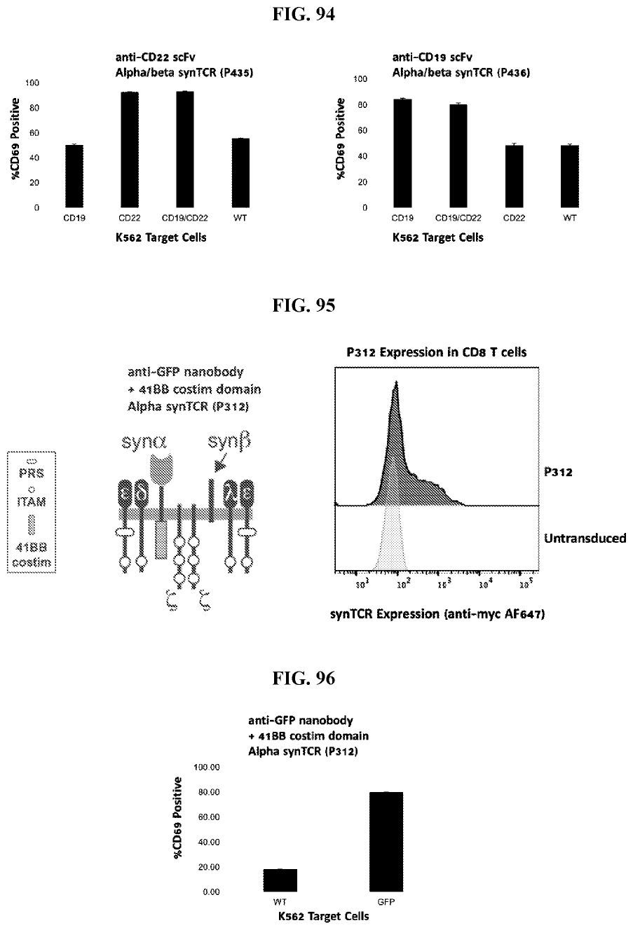

SynTCRs were designed with two separate antigen binding domains (“dual synTCRs”), i.e., with binding domains on both the alpha and beta chains, and the designed synTCRs were tested for activity. Specifically, primary human CD8 T cells were transduced with two different dual-synTCRs: an “anti-CD22 alpha/beta synTCR” (P435, anti-CD22 scFv fused to truncated TCR alpha chain paired with anti-CD22 scFv fused to truncated TCR beta chain) and an “anti-CD19 alpha/beta synTCR” (P436, anti-CD19 scFv fused to truncated TCR alpha chain paired with anti-CD19 scFv fused to truncated TCR beta chain). The synTCR-expressing CD8 T cells were co-cultured overnight with K562 target cells expressing different antigens (i.e., exogenous CD19, exogenous CD22, both exogenous CD19 and CD22 (“CD19/CD22”) or no exogenous antigen (“WT”)). After 24 hours of co-culture, T cell activation was assayed flow cytometrically by quantifying the level of CD69 expression. For both anti-CD22 alpha/beta-synTCR and anti-CD19 alpha/beta-synTCR T cells, CD69 expression was upregulated in the presence of target cells expressing the corresponding antigen (FIG. 94). Antigen-specific T cell activation driven by both anti-CD19 and anti-CD22 alpha/beta dual-synTCRs demonstrates that scFvs can be used on either or both truncated TCR alpha and TCR beta chains in synTCR designs.

The above described constructs include the following:

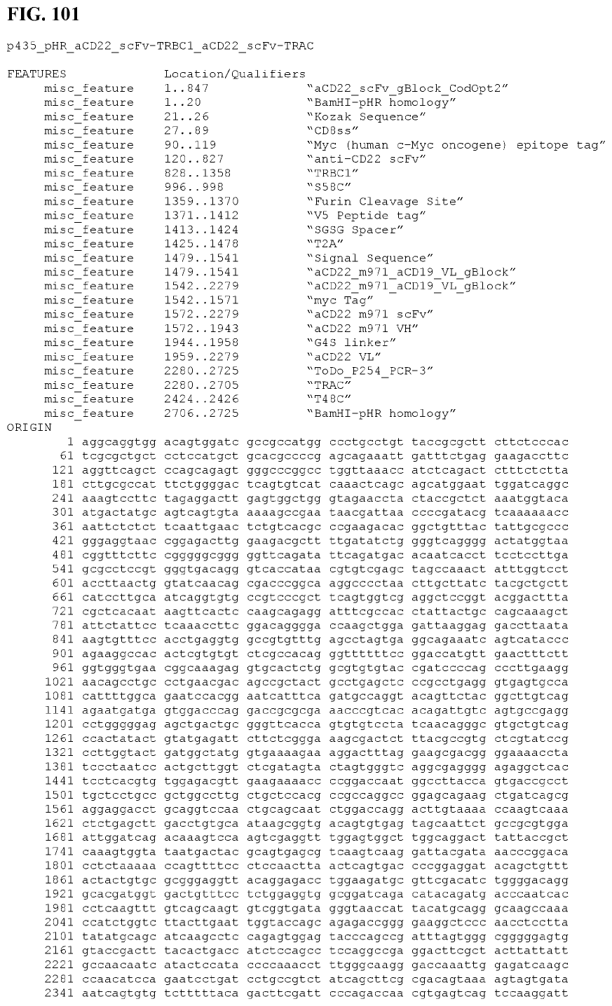

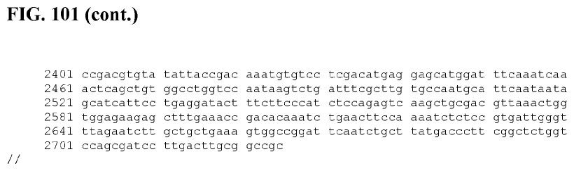

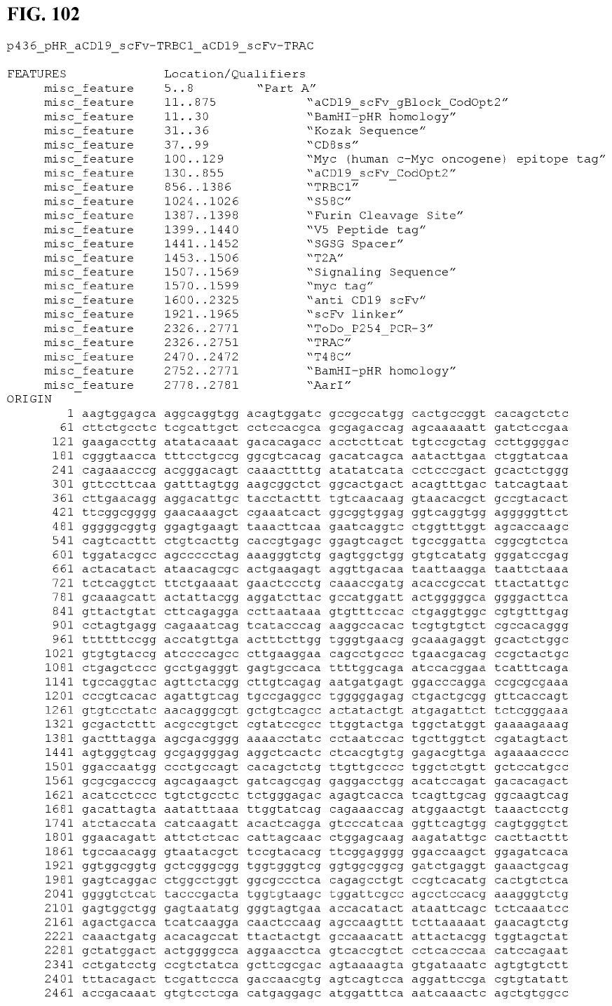

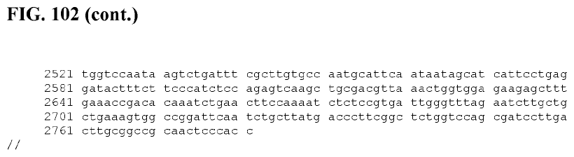

pHR_aCD22_scFv-TRBC1_aCD22_scFv-TRAC(P435), as
depicted in FIG. 101 and having the following
translated amino acid sequence:
(SEQ ID NO: 132)
MALPVTALLLPLALLLHAARPEQKLISEEDLQVQLQQSGPGLVKPSQTLS
LTCAISGDSVSSNSAAWNWIRQSPSRGLEWLGRTYYRSKWYNDYAVSVKS
RITINPDTSKNQFSLQLNSVTPEDTAVYYCAREVTGDLEDAFDIWGQGTM
VTVSSGGGGSDIQMTQSPSSLSASVGDRVTITCRASQTIWSYLNWYQQRP
GKAPNLLIYAASSLQSGVPSRFSGRGSGTDFTLTISSLQAEDFATYYCQQ
SYSIPQTFGQGTKLEIKEDLNKVFPPEVAVFEPSEAEISHTQKATLVCLA
TGFFPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGF
TSVSYQQGVLSATILYEILLGKATLYAVLVSALVLMAMVKRKDFRKRRGK
PIPNPLLGLDSTSGSGEGRGSLLTCGDVEENPGPMALPVTALLLPLALLL
HAARPEQKLISEEDLQVQLQQSGPGLVKPSQTLSLTCAISGDSVSSNSAA
WNWIRQSPSRGLEWLGRTYYRSKWYNDYAVSVKSRITINPDTSKNQFSLQ
LNSVTPEDTAVYYCAREVTGDLEDAFDIWGQGTMVTVSSGGGGSDIQMTQ
SPSSLSASVGDRVTITCRASQTIWSYLNWYQQRPGKAPNLLIYAASSLQS
GVPSRFSGRGSGTDFTLTISSLQAEDFATYYCQQSYSIPQTFGQGTKLEI
KPNIQNPDPAVYQLRDSKSSDKSVCLFTDFDSQTNVSQSKDSDVYITDKC
VLDMRSMDFKSNSAVAWSNKSDFACANAFNNSIIPEDTFFPSPESSCDVK
LVEKSFETDTNLNFQNLSVIGFRILLLKVAGFNLLMTLRLWSSDP.
pHR_aCD19_scFv-TRBC1_aCD19_scFv-TRAC(P436), as
depicted in FIG. 102 and having the following
translated amino acid sequence:
(SEQ ID NO: 133)
MALPVTALLLPLALLLHAARPEQKLISEEDLDIQMTQTTSSLSASLGDRV
TISCRASQDISKYLNWYQQKPDGTVKLLIYHTSRLHSGVPSRFSGSGSGT
DYSLTISNLEQEDIATYFCQQGNTLPYTFGGGTKLEITGGGGSGGGGSGG
GGSEVKLQESGPGLVAPSQSLSVTCTVSGVSLPDYGVSWIRQPPRKGLEW
LGVIWGSETTYYNSALKSRLTIIKDNSKSQVFLKMNSLQTDDTAIYYCAK
HYYYGGSYAMDYWGQGTSVTVSSEDLNKVFPPEVAVFEPSEAEISHTQKA
TLVCLATGFFPDHVELSWWVNGKEVHSGVCTDPQPLKEQPALNDSRYCLS
SRLRVSATFWQNPRNHFRCQVQFYGLSENDEWTQDRAKPVTQIVSAEAWG
RADCGFTSVSYQQGVLSATILYEILLGKATLYAVLVSALVLMAMVKRKDF
RKRRGKPIPNPLLGLDSTSGSGEGRGSLLTCGDVEENPGPMALPVTALLL
PLALLLHAARPEQKLISEEDLDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSPNIQNPDPAVYQLRDSKSSDKSVCLFTDFDSQTNVSQ
SKDSDVYITDKCVLDMRSMDFKSNSAVAWSNKSDFACANAFNNSIIPEDT
FFPSPESSCDVKLVEKSFETDTNLNFQNLSVIGFRILLLKVAGFNLLMTL
RLWSSDP.

### Example 5: Co-Stimulatory Domain Containing synTCRs

SynTCRs were designed with costimulatory domain(s) fused to the intracellular portion of the synTCR chain(s). For example, synTCR with the 41BB costimulatory domain fused intracellularly to the alpha chain of the anti-GFP alpha synTCR (P312) was designed and tested. Specifically, primary human CD8 T cells were transduced with the anti-GFP alpha synTCR+41BB synTCR (P312, anti-GFP nanobody fused extracellularly to truncated TCR alpha chain with intracellular 41BB fusion paired with truncated TCR beta chain). SynTCR expression was flow cytometrically assayed by measuring anti-myc staining, as the designed synTCR receptor included an N-terminal myc tag. P312 was found to be expressed in primary human CD8 T cells, as measured by increased anti-myc staining relative to untransduced T cell controls (FIG. 95).

To test the function of the P312, synTCR-expressing CD8 T cells were co-cultured overnight with WT or GFP+ K562 target cells. After 24 hours of co-culture, T cell activation was assayed flow cytometrically by quantifying the level of CD69 expression. P312 transduced T cells upregulated CD69 expression only in the presence of GFP+ K562 target cells (FIG. 96). Antigen-specific T cell activation driven by the anti-GFP alpha synTCR+41BB synTCR demonstrates the effective use of designed synTCRs that contain incorporated costimulatory domains.

The above described construct includes the following:

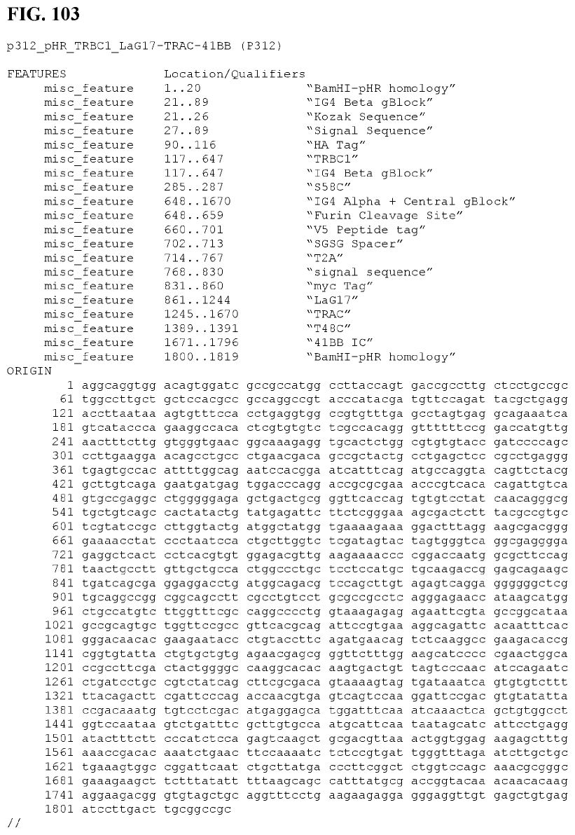

pHR_TRBC1_LaG17-TRAC-41BB (P312), as depicted in
FIG. 103 and having the following translated amino
acid sequence:
(SEQ ID NO: 134)
MALPVTALLLPLALLLHAARPYPYDVPDYAEDLNKVFPPEVAVFEPSEAE
ISHTQKATLVCLATGFFPDHVELSWWVNGKEVHSGVCTDPQPLKEQPALN
DSRYCLSSRLRVSATFWQNPRNHFRCQVQFYGLSENDEWTQDRAKPVTQI
VSAEAWGRADCGFTSVSYQQGVLSATILYEILLGKATLYAVLVSALVLMA
MVKRKDFRKRRGKPIPNPLLGLDSTSGSGEGRGSLLTCGDVEENPGPMAL
PVTALLLPLALLLHAARPEQKLISEEDLMADVQLVESGGGLVQAGGSLRL
SCAASGRTISMAAMSWFRQAPGKEREFVAGISRSAGSAVHADSVKGRFTI
SRDNTKNTLYLQMNSLKAEDTAVYYCAVRTSGFFGSIPRTGTAFDYWGQG
TQVTVSPNIQNPDPAVYQLRDSKSSDKSVCLFTDFDSQTNVSQSKDSDVY
ITDKCVLDMRSMDFKSNSAVAWSNKSDFACANAFNNSIIPEDTFFPSPES
SCDVKLVEKSFETDTNLNFQNLSVIGFRILLLKVAGFNLLMTLRLWSSKR
GRKKLLYIFKQPFMRPVQTTQEEDGCSCRFPEEEEGGCEL.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A nucleic acid encoding a chimeric T cell antigen receptor (TCR) comprising a modified α-chain and a modified β-chain that, when present in an immune cell membrane, activates the immune cell when the chimeric TCR binds an antigen, wherein:
a) the modified α-chain is a fusion polypeptide comprising a heterologous antigen-binding domain, that specifically binds the antigen, fused to the extracellular domain of a TCR α-chain; or
b) the modified β-chain is a fusion polypeptide comprising a heterologous antigen-binding domain, that specifically binds the antigen, fused to the extracellular domain of a TCR β-chain.

2. The nucleic acid according to claim 1, wherein the antigen is a cancer antigen.

3. The nucleic acid according to claim 1 or 2, wherein the antigen is a cell surface antigen.

4. The nucleic acid according to claim 1 or 2, wherein the antigen is a peptide-major histocompatibility complex (peptide-MHC).

5. The nucleic acid according to any of the preceding claims, wherein the heterologous antigen-binding domain comprises an antibody.

6. The nucleic acid according to claim 5, wherein the antibody is a scFv or a single domain antibody.

7. The nucleic acid according to any of claims 1 to 3, wherein the heterologous antigen-binding domain comprises a ligand binding domain of a receptor.

8. The nucleic acid according to any of the preceding claims, wherein the heterologous antigen-binding domain is fused directly to the extracellular domain.

9. The nucleic acid according to any of claims 1 to 7, wherein the heterologous antigen-binding domain is fused to the extracellular domain by a linker.

10. The nucleic acid according to claim 9, wherein the linker is less than 30 amino acids in length.

11. The nucleic acid according to claim 10, wherein the linker is less than 20 amino acids in length.

12. The nucleic acid according to any of the preceding claims, wherein the modified α-chain comprises a truncated α-chain, the modified β-chain comprises a truncated β-chain or the modified α-chain comprises a truncated α-chain and the modified β-chain comprises a truncated β-chain.

13. The nucleic acid according to claim 12, wherein the modified α-chain, the modified β-chain or both the modified α-chain and the modified β-chain do not comprise a variable region.

14. The nucleic acid according to claim 12 or 13, wherein the extracellular domain to which the heterologous antigen-binding domain is fused is a constant region of the TCR α-chain or the TCR β-chain.

15. The nucleic acid according to claim 14, wherein the heterologous antigen-binding domain is fused directly to the constant region.

16. The nucleic acid according to claim 14, wherein the heterologous antigen-binding domain is fused to the constant region by a linker.

17. The nucleic acid according to claim 16, wherein the linker is less than 30 amino acids in length.

18. The nucleic acid according to claim 17, wherein the linker is less than 20 amino acids in length.

19. The nucleic acid according to any of the preceding claims, wherein the chimeric TCR comprises a recombinant disulfide bond between an α-chain cysteine mutation and a β-chain cysteine mutation.

20. The nucleic acid according to claim 19, wherein the α-chain cysteine mutation is a T48C mutation and the β-chain cysteine mutation is a S57C mutation.

21. The nucleic acid according to any of the preceding claims, wherein the modified α-chain and the modified β-chain are domain swapped modified α- and β-chains.

22. The nucleic acid according to claim 21, wherein the domain swapped modified α- and β-chains comprise swapped α- and β-chain transmembrane regions.

23. The nucleic acid according to claim 21 or 22, wherein the domain swapped modified α- and β-chains comprise swapped α- and β-chain cytoplasmic regions.

24. The nucleic acid according to any of claims 21 to 23, wherein the domain swapped modified α- and β-chains comprise swapped α- and β-chain connecting regions.

25. The nucleic acid according to any of the preceding claims, wherein the modified α-chain is a fusion polypeptide comprising two or more heterologous antigen-binding domains, that each specifically bind a different antigen, fused to the extracellular domain of a TCR α-chain.

26. The nucleic acid according to claim 25, wherein the fusion polypeptide comprises a first heterologous antigen-binding domain fused to the extracellular domain of a TCR α-chain and a second heterologous antigen-binding domain fused to the first heterologous antigen-binding domain.

27. The nucleic acid according to any of the preceding claims, wherein the modified β-chain is a fusion polypeptide comprising two or more heterologous antigen-binding domains, each of which specifically binds a different antigen, fused to the extracellular domain of a TCR β-chain.

28. The nucleic acid according to claim 27, wherein the fusion polypeptide comprises a first heterologous antigen-binding domain fused to the extracellular domain of a TCR β-chain and a second heterologous antigen-binding domain fused to the first heterologous antigen-binding domain.

29. The nucleic acid according to any of the preceding claims, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain.

30. The nucleic acid according to any of the preceding claims, wherein the modified α-chain, the modified β-chain, or both the modified α-chain and the modified β-chain comprise a costimulatory domain.

31. The nucleic acid according to any of the preceding claims, wherein the chimeric TCR activates the immune cell to exhibit cytotoxic activity to a target cell expressing the antigen.

32. The nucleic acid according to claim 31, wherein the activated immune cell results in a 10% or greater increase in killing of the target cell as compared to a control immune cell without the chimeric TCR.

33. The nucleic acid according to any of the preceding claims, wherein the modified α-chain and the modified β-chain are linked into a single chain by a linking polypeptide comprising a transmembrane domain.

34. A recombinant expression vector comprising the nucleic acid according to any of claims 1 to 33, wherein the expression vector comprises a promoter operably linked to a nucleotide sequence encoding the modified α-chain and a nucleotide sequence encoding the modified β-chain.

35. The expression vector according to claim 34, wherein the expression vector comprises a bicistronic-facilitating sequence between the nucleotide sequence encoding the modified α-chain and the nucleotide sequence encoding the modified β-chain.

36. The expression vector according to claim 35, wherein the bicistronic-facilitating sequence comprises a furin cleavage site encoding sequence, an amino acid spacer encoding sequence and a 2A peptide encoding sequence.

37. The expression vector according to claim 36, wherein the amino acid spacer encoding sequence comprises a nucleotide sequence encoding a V5 peptide.

38. The expression vector according to any of claims 34 to 37, wherein the promoter is an inducible or conditional promoter.

39. A recombinant expression vector comprising the nucleic acid according to any of claims 1 to 33, wherein the recombinant expression vector comprises a first promoter operably linked to a nucleotide sequence encoding the modified α-chain and a second promoter operably linked to a nucleotide sequence encoding the modified β-chain.

40. The expression vector according to claim 39, wherein the first promoter is an inducible or conditional promoter.

41. The expression vector according to claim 39 or 40, wherein the second promoter is an inducible or conditional promoter.

42. The expression vector according to any of claims 39 to 41, wherein the first promoter and the second promoter are copies of the same promoter.

43. An immune cell comprising the expression vector according to any of claims 34 to 42.

44. An immune cell genetically modified to comprise the nucleic acid according to any of claims 1 to 33.

45. A method of killing a target cell, the method comprising contacting the target cell with the immune cell according to claim 43 or 44, wherein the target cell expresses the antigen to which the chimeric TCR binds.

46. The method according to claim 45, wherein the method is performed in vitro and the contacting comprises co-culturing the target cell and the immune cell.

47. The method according to claim 45, wherein the method is performed in vivo and the contacting comprises administering the immune cell to a subject having the target cell.

48. The method according to claim 47, wherein the target cell is a cancer cell and the method comprises administering to the subject an amount of the immune cells effective to treat the subject for the cancer.

49. A nucleic acid encoding a modified T cell antigen receptor (TCR) α-chain that, when present in a chimeric TCR within an immune cell membrane, activates the immune cell when the chimeric TCR binds an antigen, the modified TCR α-chain comprising:
a heterologous antigen-binding domain;
a truncated TCR α-chain extracellular domain linked to the heterologous antigen-binding domain;
a TCR chain connecting region linked to the truncated TCR α-chain;
a TCR chain transmembrane domain linked to the TCR chain connecting region; and
a TCR chain cytoplasmic domain.

50. The nucleic acid according to claim 49, wherein the antigen is a cancer antigen.

51. The nucleic acid according to claim 49 or 50, wherein the antigen is a cell surface antigen.

52. The nucleic acid according to claim 49 or 50, the antigen is a peptide-major histocompatibility complex (peptide-MHC).

53. The nucleic acid according to any of claims 49 to 52, wherein the heterologous antigen-binding domain comprises an antibody.

54. The nucleic acid according to claims 53, wherein the antibody is a scFv or a single domain antibody.

55. The nucleic acid according to any of claims 49 to 51, wherein the heterologous antigen-binding domain comprises a ligand binding domain of a receptor.

56. The nucleic acid according to any of claims 49 to 55, wherein the heterologous antigen-binding domain is linked directly to the truncated TCR α-chain extracellular domain.

57. The nucleic acid according to any of claims 49 to 55, wherein the heterologous antigen-binding domain is linked to the truncated TCR α-chain extracellular domain by a linker.

58. The nucleic acid according to claim 57, wherein the linker is less than 30 amino acids in length.

59. The nucleic acid according to claims 58, wherein the linker is less than 20 amino acids in length.

60. The nucleic acid according to any of claims 49 to 59, wherein the truncated TCR α-chain extracellular domain does not comprise a variable region.

61. The nucleic acid according to any of claims 49 to 60, wherein the TCR chain connecting region comprises one or more cysteine substitutions.

62. The nucleic acid according to claim 61, wherein the TCR chain connecting region is a TCR α-chain connecting region.

63. The nucleic acid according to claim 62, wherein the one or more cysteine substitutions comprise a T48C mutation.

64. The nucleic acid according to claim 61, wherein the TCR chain connecting region is a TCR β-chain connecting region.

65. The nucleic acid according to claim 64, wherein the one or more cysteine substitutions comprise a S57C mutation.

66. The nucleic acid according to any of claims 49 to 65, wherein the TCR chain transmembrane domain is a TCR α-chain transmembrane domain.

67. The nucleic acid according to any of claims 49 to 65, wherein the TCR chain transmembrane domain is a TCR β-chain transmembrane domain.

68. The nucleic acid according to any of claims 49 to 67, wherein the TCR chain cytoplasmic domain is a TCR α-chain cytoplasmic domain.

69. The nucleic acid according to any of claims 49 to 68, wherein the TCR chain cytoplasmic domain is a TCR β-chain cytoplasmic domain.

70. The nucleic acid according to any of claims 49 to 69, wherein the modified TCR α-chain comprises two different heterologous antigen-binding domains.

71. The nucleic acid according to any of claims 49 to 70, wherein the modified TCR α-chain further comprises a costimulatory domain.

72. The nucleic acid according to any of claims 49 to 71, wherein the chimeric TCR comprising the modified TCR α-chain activates the immune cell to exhibit cytotoxic activity to a target cell expressing the antigen.

73. The nucleic acid according to claim 72,wherein the activated immune cell results in a 10% or greater increase in killing of the target cell as compared to a control immune cell without the chimeric TCR.

74. A recombinant expression vector comprising the nucleic acid according to any of claims 49 to 73.

75. An immune cell comprising the expression vector of claim 74.

76. An immune cell genetically modified to comprise the nucleic acid according to any of claims 49 to 73.

77. An immune cell comprising:
a first nucleic acid encoding a modified TCR α-chain comprising:
a heterologous antigen-binding domain linked to a TCR α-chain; and
a first cysteine substitution within the chain connecting region of the TCR α-chain; and
a second nucleic acid encoding a modified TCR β-chain comprising a second cysteine substitution, wherein the first and second cysteine substitutions result in a recombinant disulfide bond between the modified TCR α-chain and the modified TCR β-chain.

78. The immune cell according to claim 77, wherein the first cysteine substitution is a T48C mutation and the second cysteine substitution is a S57C mutation.

79. A method of killing a target cell, the method comprising contacting the target cell with an immune cell according to any of claims 75 to 78, wherein the target cell expresses the antigen to which the chimeric TCR binds.

80. The method according to claim 79, wherein the method is performed in vitro and the contacting comprises co-culturing the target cell and the immune cell.

81. The method according to claim 79, wherein the method is performed in vivo and the contacting comprises administering the immune cell to a subject having the target cell.

82. The method according to claim 81, wherein the target cell is a cancer cell and the method comprises administering to the subject an amount of the immune cells effective to treat the subject for the cancer.

83. A nucleic acid encoding a modified T cell antigen receptor (TCR) β-chain that, when present in a chimeric TCR within an immune cell membrane, activates the immune cell when the chimeric TCR binds an antigen, the modified TCR β-chain comprising:
a heterologous antigen-binding domain;
a truncated TCR β-chain extracellular domain linked to the heterologous antigen-binding domain;
a TCR chain connecting region linked to the truncated TCR β-chain;
a TCR chain transmembrane domain linked to the TCR chain connecting region; and
a TCR chain cytoplasmic domain.

84. The nucleic acid according to claim 83, wherein the antigen is a cancer antigen.

85. The nucleic acid according to claim 83 or 84, wherein the antigen is a cell surface antigen.

86. The nucleic acid according to claim 83 or 84, the antigen is a peptide-major histocompatibility complex (peptide-MHC).

87. The nucleic acid according to any of claims 83 to 86, wherein the heterologous antigen-binding domain comprises an antibody.

88. The nucleic acid according to any of claim 87, wherein the antibody is a scFv or a single domain antibody.

89. The nucleic acid according to any of claims 83 to 85, wherein the heterologous antigen-binding domain comprises a ligand binding domain of a receptor.

90. The nucleic acid according to any of claims 83 to 89, wherein the heterologous antigen-binding domain is linked directly to the truncated TCR β-chain extracellular domain.

91. The nucleic acid according to any of claims 83 to 89, wherein the heterologous antigen-binding domain is linked to the truncated TCR β-chain extracellular domain by a linker.

92. The nucleic acid according to claim 91, wherein the linker is less than 30 amino acids in length.

93. The nucleic acid according to claim 92, wherein the linker is less than 20 amino acids in length.

94. The nucleic acid according to any of claims 83 to 93, wherein the truncated TCR β-chain extracellular domain does not comprise a variable region.

95. The nucleic acid according to any of claims 83 to 94, wherein the TCR chain connecting region comprises one or more cysteine substitutions.

96. The nucleic acid according to claim 95, wherein the TCR chain connecting region is a TCR β-chain connecting region.

97. The nucleic acid according to claim 96, wherein the one or more cysteine substitutions comprise a S57C mutation.

98. The nucleic acid according to claim 95, wherein the TCR chain connecting region is a TCR α-chain connecting region.

99. The nucleic acid according to claim 98, wherein the one or more cysteine substitutions comprise a T48C mutation.

100. The nucleic acid according to any of claims 83 to 99, wherein the TCR chain transmembrane domain is a TCR β-chain transmembrane domain.

101. The nucleic acid according to any of claims 83 to 99, wherein the TCR chain transmembrane domain is a TCR α-chain transmembrane domain.

102. The nucleic acid according to any of claims 83 to 101, wherein the TCR chain cytoplasmic domain is a TCR β-chain cytoplasmic domain.

103. The nucleic acid according to any of claims 83 to 101, wherein the TCR chain cytoplasmic domain is a TCR α-chain cytoplasmic domain.

104. The nucleic acid according to any of claims 83 to 103, wherein the modified TCR β-chain comprises two different heterologous antigen-binding domains.

105. The nucleic acid according to any of claims 83 to 104, wherein the modified TCR β-chain further comprises a costimulatory domain.

106. The nucleic acid according to any of claims 83 to 105, wherein the chimeric TCR comprising the modified TCR β-chain activates the immune cell to exhibit cytotoxic activity to a target cell expressing the antigen.

107. The nucleic acid according to claim 106, wherein the activated immune cell results in a 10% or greater increase in killing of the target cell as compared to a control immune cell without the chimeric TCR.

108. A recombinant expression vector comprising the nucleic acid according to any of claims 83 to 107.

109. An immune cell comprising the expression vector of claim 108.

110. An immune cell genetically modified to comprise the nucleic acid according to any of claims 83 to 107.

111. An immune cell comprising:
a first nucleic acid encoding a modified TCR β-chain comprising:
a heterologous antigen-binding domain linked to a TCR β-chain; and
a first cysteine substitution within the chain connecting region of the TCR β-chain; and
a second nucleic acid encoding a modified TCR α-chain comprising a second cysteine substitution, wherein the first and second cysteine substitutions result in a recombinant disulfide bond between the modified TCR β-chain and the modified TCR α-chain.

112. The immune cell according to claim 111, wherein the first cysteine substitution is a S57C mutation and the second cysteine substitution is a T48C mutation.

113. A method of killing a target cell, the method comprising contacting the target cell with an immune cell according to any of claims 109 to 112, wherein the target cell expresses the antigen to which the chimeric TCR binds.

114. The method according to claim 113, wherein the method is performed in vitro and the contacting comprises co-culturing the target cell and the immune cell.

115. The method according to claim 113, wherein the method is performed in vivo and the contacting comprises administering the immune cell to a subject having the target cell.

116. The method according to claim 115, wherein the target cell is a cancer cell and the method comprises administering to the subject an amount of the immune cells effective to treat the subject for the cancer.

117. A method of treating a subject for a condition, the method comprising:
administering to the subject an effective amount of the immune cells according to any of claims 43, 44, 75-78 and 109-112 in combination with an agent that ameliorates at least one side effect of the immune cells.

118. The method according to claim 117, wherein the condition is cancer.

119. A method of treating a subject for cancer, the method comprising:
administering to the subject an effective amount of the immune cells according to any of claims 43, 44, 75-78 and 109-112 in combination with a conventional cancer therapy.

120. The method according to claim 119, wherein the immune cells and the conventional cancer therapy are administered in combination with an agent that ameliorates at least one side effect of the immune cells.

121. A chimeric T cell antigen receptor (TCR) comprising a modified α-chain and a modified β-chain that, when present in an immune cell membrane, activates the immune cell when the chimeric TCR binds an antigen, wherein:
a) the modified α-chain is a fusion polypeptide comprising a heterologous antigen-binding domain, that specifically binds the antigen, fused to the extracellular domain of a TCR α-chain; or
b) the modified β-chain is a fusion polypeptide comprising a heterologous antigen-binding domain, that specifically binds the antigen, fused to the extracellular domain of a TCR β-chain.

122. The chimeric TCR according to claim 121, wherein the antigen is a cancer antigen.

123. The chimeric TCR according to claim 121 or 122, wherein the antigen is a cell surface antigen.

124. The chimeric TCR according to claim 121 or 122, wherein the antigen is a peptide-major histocompatibility complex (peptide-MHC).

125. The chimeric TCR according to any of claims 121 to 124, wherein the heterologous antigen-binding domain comprises an antibody.

126. The chimeric TCR according to claim 125, wherein the antibody is a scFv or a single domain antibody.

127. The chimeric TCR according to any of claims 121 to 123, wherein the heterologous antigen-binding domain comprises a ligand binding domain of a receptor.

128. The chimeric TCR according to any of claims 121 to 127, wherein the heterologous antigen-binding domain is fused directly to the extracellular domain.

129. The chimeric TCR according to any of claims 121 to 127, wherein the heterologous antigen-binding domain is fused to the extracellular domain by a linker.

130. The chimeric TCR according to claim 129, wherein the linker is less than 30 amino acids in length.

131. The chimeric TCR according to claim 130, wherein the linker is less than 20 amino acids in length.

132. The chimeric TCR according to any of claims 121 to 131, wherein the modified α-chain comprises a truncated α-chain, the modified β-chain comprises a truncated β-chain or the modified α-chain comprises a truncated α-chain and the modified β-chain comprises a truncated β-chain.

133. The chimeric TCR according to claim 132, wherein the modified α-chain, the modified β-chain or both the modified α-chain and the modified β-chain do not comprise a variable region.

134. The chimeric TCR according to claim 132 or 133, wherein the extracellular domain to which the heterologous antigen-binding domain is fused is a constant region of the TCR α-chain or the TCR β-chain.

135. The chimeric TCR according to claim 134, wherein the heterologous antigen-binding domain is fused directly to the constant region.

136. The chimeric TCR according to claim 134, wherein the heterologous antigen-binding domain is fused to the constant region by a linker.

137. The chimeric TCR according to claim 136, wherein the linker is less than 30 amino acids in length.

138. The chimeric TCR according to claim 137, wherein the linker is less than 20 amino acids in length.

139. The chimeric TCR according to any of claims 121 to 138, wherein the chimeric TCR comprises a recombinant disulfide bond between a α-chain cysteine mutation and a β-chain cysteine mutation.

140. The chimeric TCR according to claim 139, wherein the α-chain cysteine mutation is a T48C mutation and the β-chain cysteine mutation is a S57C mutation.

141. The chimeric TCR according to any of claims 121 to 140, wherein the modified α-chain and the modified β-chain are domain swapped modified α- and β-chains.

142. The chimeric TCR according to claim 141, wherein the domain swapped modified α- and β-chains comprise swapped α- and β-chain transmembrane regions.

143. The chimeric TCR according to claim 141 or 142, wherein the domain swapped modified α- and β-chains comprise swapped α- and β-chain cytoplasmic regions.

144. The chimeric TCR according to any of claims 141 to 143, wherein the domain swapped modified α- and β-chains comprise swapped α- and β-chain connecting regions.

145. The chimeric TCR according to any of claims 121 to 144, wherein the modified α-chain is a fusion polypeptide comprising two or more heterologous antigen-binding domains, that each specifically bind a different antigen, fused to the extracellular domain of a TCR α-chain.

146. The chimeric TCR according to claim 145, wherein the fusion polypeptide comprises a first heterologous antigen-binding domain fused to the extracellular domain of a TCR α-chain and a second heterologous antigen-binding domain fused to the first heterologous antigen-binding domain.

147. The chimeric TCR according to any of claims 121 to 146, wherein the modified β-chain is a fusion polypeptide comprising two or more heterologous antigen-binding domains, that each specifically bind a different antigen, fused to the extracellular domain of a TCR β-chain.

148. The chimeric TCR according to claim 147, wherein the fusion polypeptide comprises a first heterologous antigen-binding domain fused to the extracellular domain of a TCR β-chain and a second heterologous antigen-binding domain fused to the first heterologous antigen-binding domain.

149. The chimeric TCR according to any of claims 121 to 148, wherein the modified α-chain is a fusion polypeptide comprising one or more heterologous antigen-binding domains fused to the extracellular domain of a TCR α-chain and the modified β-chain is a fusion polypeptide comprising one or more heterologous antigen-binding domains fused to the extracellular domain of the TCR β-chain.

150. The chimeric TCR according to any of claims 121 to 149, wherein the modified α-chain, the modified β-chain, or both the modified α-chain and the modified β-chain comprise a costimulatory domain.

151. The chimeric TCR according to any of claims 121 to 150, wherein the chimeric TCR activates the immune cell to exhibit cytotoxic activity to a target cell expressing the antigen.

152. The chimeric TCR according to claim 151, wherein the activated immune cell results in a 10% or greater increase in killing of the target cell as compared to a control immune cell without the chimeric TCR.

153. The chimeric TCR according to any of claims 121 to 152, wherein the modified α-chain and the modified β-chain are linked into a single chain by a linking polypeptide comprising a transmembrane domain.

154. A method of killing a target cell, the method comprising contacting the target cell with an immune cell expressing a chimeric TCR according to any of claims 149 to 153, wherein the modified α-chain comprises a heterologous antigen-binding domain specific for a first antigen expressed by the target cell and the modified β-chain comprises a heterologous antigen-binding domain specific for a second antigen expressed by the target cell.

155. The method according to claim 154, wherein the first antigen expressed by the target cell and the second antigen expressed by the target cell are the same antigen.

156. The method according to claim 155, wherein the heterologous antigen-binding domain of the modified α-chain and the heterologous antigen-binding domain of the modified β-chain are the same heterologous antigen-binding domain.

157. The method according to claim 155, wherein the heterologous antigen-binding domain of the modified α-chain and the heterologous antigen-binding domain of the modified β-chain are different heterologous antigen-binding domains.

158. The method according to claim 154, wherein the first antigen expressed by the target cell and the second antigen expressed by the target cell are different antigens.


SEQUENCE LISTING (245 sequences)

SEQ ID NO: 1 - protein, 133 aa, Artificial Sequence
  synthethic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 2 - protein, 132 aa, Artificial Sequence
  synthethic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 3 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 138 aa, Mus musculus
PYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 5 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 6 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 7 - protein, 20 aa, Artificial Sequence
  synthethic polypeptide
CDVKLVEKSFETDTNLNFQN

SEQ ID NO: 8 - protein, 20 aa, Artificial Sequence
  synthethic polypeptide
VIGFRILLLKVAGFNLLMTL

SEQ ID NO: 9 - protein, 16 aa, Artificial Sequence
  synthethic polypeptide
CGFTSVSYQQGVLSAT

SEQ ID NO: 10 - protein, 21 aa, Artificial Sequence
  synthethic polypeptide
ILLGKATLYAVLVSALVLMAM

SEQ ID NO: 11 - protein, 142 aa, Homo sapiens
PNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 138 aa, Mus musculus
PYIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 13 - protein, 177 aa, Homo sapiens
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 14 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 15 - protein, 130 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 16 - protein, 47 aa, Homo sapiens
CDVKLVEKSFETDTNLNFQNLSVIGFRILLLKVAGFNLLMTLRLWSS

SEQ ID NO: 17 - protein, 95 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 18 - protein, 47 aa, Homo sapiens
CGFTSVSYQQGVLSATILYEILLGKATLYAVLVSALVLMAMVKRKDF

SEQ ID NO: 19 - protein, 177 aa, Artificial Sequence
  synthethic polypeptide
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSS

SEQ ID NO: 20 - protein, 142 aa, Artificial Sequence
  synthethic polypeptide
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 21 - protein, 145 aa, Homo sapiens
EDLNKVFPPEVAVFEPSEAEISHTQKATLVCLATGFFPDHVELSWWVNGKEVHSGVSTDPQPLKEQALNDSRYCLSSRLRVSATFWQNPRNHFRCQVQFYGLSENDEWTQDRAKPVTQIVSAEAWGRADCGFTSVSYQQGVLSAT

SEQ ID NO: 22 - protein, 114 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQ

SEQ ID NO: 23 - protein, 31 aa, Artificial Sequence
  synthethic polypeptide
ILYEILLGKATLYAVLVSALVLMAMVKRKDF

SEQ ID NO: 24 - protein, 173 aa, Artificial Sequence
  synthethic polypeptide
EDLNKVFPPEVAVFEPSEAEISHTQKATLVCLATGFFPDHVELSWWVNGKEVHSGVSTDPQPLKEQALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSS

SEQ ID NO: 25 - protein, 145 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 26 - protein, 24 aa, Artificial Sequence
  synthethic polypeptide
IYIWAPLAGTCGVLLLSLVITLYC

SEQ ID NO: 27 - protein, 23 aa, Artificial Sequence
  synthethic polypeptide
LGLLVAGVLVLLVSLGVAIHLCC

SEQ ID NO: 28 - protein, 25 aa, Artificial Sequence
  synthethic polypeptide
ALIVLGGVAGLLLFIGLGIFFCVRC

SEQ ID NO: 29 - protein, 23 aa, Artificial Sequence
  synthethic polypeptide
LCYLLDGILFIYGVILTALFLRV

SEQ ID NO: 30 - protein, 26 aa, Artificial Sequence
  synthethic polypeptide
WVLVVVGGVLACYSLLVTVAFIIFWV

SEQ ID NO: 31 - protein, 26 aa, Artificial Sequence
  synthethic polypeptide
VAAILGLGLVLGLLGPLAILLALYLL

SEQ ID NO: 32 - protein, 24 aa, Artificial Sequence
  synthethic polypeptide
ALPAALAVISFLLGLGLGVACVLA

SEQ ID NO: 33 - protein, 5 aa, Artificial Sequence
  synthethic polypeptide
GSGGS

SEQ ID NO: 34 - protein, 4 aa, Artificial Sequence
  synthethic polypeptide
GGGS

SEQ ID NO: 35 - protein, 4 aa, Artificial Sequence
  synthethic polypeptide
GGSG

SEQ ID NO: 36 - protein, 5 aa, Artificial Sequence
  synthethic polypeptide
GGSGG

SEQ ID NO: 37 - protein, 5 aa, Artificial Sequence
  synthethic polypeptide
GSGSG

SEQ ID NO: 38 - protein, 5 aa, Artificial Sequence
  synthethic polypeptide
GSGGG

SEQ ID NO: 39 - protein, 5 aa, Artificial Sequence
  synthethic polypeptide
GGGSG

SEQ ID NO: 40 - protein, 5 aa, Artificial Sequence
  synthethic polypeptide
GSSSG

SEQ ID NO: 41 - protein, 15 aa, Artificial Sequence
  synthethic polypeptide
GGGGSGGGGSGGGGS

SEQ ID NO: 42 - protein, 4 aa, Artificial Sequence
  synthethic polypeptide
SGSG

SEQ ID NO: 43 - protein, 17 aa, Artificial Sequence
  synthethic polypeptide
GSADDAKKDAAKKDGKS

SEQ ID NO: 44 - protein, 5 aa, Artificial Sequence
  synthethic polypeptide
GGGGS

SEQ ID NO: 45 - protein, 9 aa, Artificial Sequence
  synthethic polypeptide
RMFPNAPYL

SEQ ID NO: 46 - protein, 10 aa, Artificial Sequence
  synthethic polypeptide
KLVVVGAGGV

SEQ ID NO: 47 - protein, 10 aa, Artificial Sequence
  synthethic polypeptide
KLVVVGAVGV

SEQ ID NO: 48 - protein, 10 aa, Artificial Sequence
  synthethic polypeptide
KLVVVGACGV

SEQ ID NO: 49 - protein, 10 aa, Artificial Sequence
  synthethic polypeptide
KLVVVGADGV

SEQ ID NO: 50 - protein, 9 aa, Artificial Sequence
  synthethic polypeptide
VVGAVGVGK

SEQ ID NO: 51 - protein, 9 aa, Artificial Sequence
  synthethic polypeptide
VVGACGVGK

SEQ ID NO: 52 - protein, 9 aa, Artificial Sequence
  synthethic polypeptide
VVGAGGVGK

SEQ ID NO: 53 - protein, 9 aa, Artificial Sequence
  synthethic polypeptide
KITDFGLAK

SEQ ID NO: 54 - protein, 9 aa, Artificial Sequence
  synthethic polypeptide
KITDFGRAK

SEQ ID NO: 55 - protein, 9 aa, Artificial Sequence
  synthethic polypeptide
VLQELNVTV

SEQ ID NO: 56 - protein, 9 aa, Artificial Sequence
  synthethic polypeptide
EADPTGHSY

SEQ ID NO: 57 - protein, 9 aa, Artificial Sequence
  synthethic polypeptide
LLGRNSFEV

SEQ ID NO: 58 - protein, 10 aa, Artificial Sequence
  synthethic polypeptide
STTPPPGTRV

SEQ ID NO: 59 - protein, 10 aa, Artificial Sequence
  synthethic polypeptide
ELAGIGILTV

SEQ ID NO: 60 - protein, 9 aa, Artificial Sequence
  synthethic polypeptide
IMDQVPFSV

SEQ ID NO: 61 - protein, 9 aa, Artificial Sequence
  synthethic polypeptide
NLVPMVATV

SEQ ID NO: 62 - protein, 9 aa, Artificial Sequence
  synthethic polypeptide
AIIRILQQL

SEQ ID NO: 63 - protein, 9 aa, Artificial Sequence
  synthethic polypeptide
VLHDDLLEA

SEQ ID NO: 64 - protein, 9 aa, Artificial Sequence
  synthethic polypeptide
VLRDDLLEA

SEQ ID NO: 65 - protein, 9 aa, Artificial Sequence
  synthethic polypeptide
SLLMWITQV

SEQ ID NO: 66 - protein, 10 aa, Artificial Sequence
  synthethic polypeptide
LLDFVRFMGV

SEQ ID NO: 67 - protein, 9 aa, Artificial Sequence
  synthethic polypeptide
FMNKFIYEI

SEQ ID NO: 68 - protein, 9 aa, Artificial Sequence
  synthethic polypeptide
KIFGSLAFL

SEQ ID NO: 69 - protein, 9 aa, Artificial Sequence
  synthethic polypeptide
GVLPALPQV

SEQ ID NO: 70 - protein, 9 aa, Artificial Sequence
  synthethic polypeptide
FLLTRILTI

SEQ ID NO: 71 - protein, 236 aa, Mus musculus
SGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKAS

SEQ ID NO: 72 - protein, 4 aa, Artificial Sequence
  synthethic polypeptide
RKRR

SEQ ID NO: 73 - protein, 18 aa, Artificial Sequence
  synthethic polypeptide
EGRGSLLTCGDVEENPGP

SEQ ID NO: 74 - protein, 14 aa, Artificial Sequence
  synthethic polypeptide
GKPIPNPLLGLDST

SEQ ID NO: 75 - protein, 21 aa, Artificial Sequence
  synthethic polypeptide
MALPVTALLLPLALLLHAARP

SEQ ID NO: 76 - protein, 5 aa, Artificial Sequence
  synthethic polypeptide
HHHHH

SEQ ID NO: 77 - protein, 6 aa, Artificial Sequence
  synthethic polypeptide
HHHHHH

SEQ ID NO: 78 - protein, 10 aa, Artificial Sequence
  synthethic polypeptide
EQKLISEEDL

SEQ ID NO: 79 - protein, 8 aa, Artificial Sequence
  synthethic polypeptide
DYKDDDDK

SEQ ID NO: 80 - protein, 8 aa, Artificial Sequence
  synthethic polypeptide
WSHPQFEK

SEQ ID NO: 81 - protein, 9 aa, Artificial Sequence
  synthethic polypeptide
YPYDVPDYA

SEQ ID NO: 82 - protein, 5 aa, Artificial Sequence
  synthethic polypeptide
RYIRS

SEQ ID NO: 83 - protein, 4 aa, Artificial Sequence
  synthethic polypeptide
FHHT

SEQ ID NO: 84 - protein, 17 aa, Artificial Sequence
  synthethic polypeptide
WEAAAREACCRECCARA

SEQ ID NO: 85 - protein, 28 aa, Artificial Sequence
  synthetic polypeptide sequence
NLSVIGFRILLLKVAGFNLLMTLRLWSS

SEQ ID NO: 86 - protein, 4 aa, Artificial Sequence
  synthetic polypeptide sequence
  misc_feature (2)..(3): Xaa can be any naturally occurring amino acid
RXXR

SEQ ID NO: 87 - protein, 4 aa, Artificial Sequence
  synthetic polypeptide sequence
  misc_feature (2)..(2): Xaa can be any naturally occurring amino acid
  MISC_FEATURE (3)..(3): Lys or Arg
  MISC_FEATURE (3)..(3): The amino acid at this position is Lys or Arg
RXXR

SEQ ID NO: 88 - protein, 6 aa, Artificial Sequence
  synthetic polypeptide sequence
  misc_feature (2)..(3): Xaa can be any naturally occurring amino acid
  misc_feature (5)..(5): Xaa can be any naturally occurring amino acid
RXXRXR

SEQ ID NO: 89 - protein, 6 aa, Artificial Sequence
  synthetic polypeptide sequence
  misc_feature (2)..(2): Xaa can be any naturally occurring amino acid
  MISC_FEATURE (3)..(3): The amino acid at this position is Lys or Arg
  misc_feature (5)..(5): Xaa can be any naturally occurring amino acid
RXXRXR

SEQ ID NO: 90 - protein, 769 aa, artificial sequence
  synthetic polypeptide sequence
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGSADDAKKDAAKKDGKSMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 91 - protein, 767 aa, artificial sequence
  synthetic polypeptide sequence
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGGGGSGGGGSGGGGSMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 548 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 93 - protein, 548 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 94 - protein, 540 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNSRKRRGKPIPNPLLGLDSTSGSGEGRGSLLTCGDVEENPGPMALPVTALLLPLALLLHAARPYPYDVPDYAPYIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 95 - protein, 540 aa, artificial sequence
  synthetic polypeptide sequence
MALPVTALLLPLALLLHAARPYPYDVPDYAEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNSRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPYIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 96 - protein, 336 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 97 - protein, 301 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 98 - protein, 332 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 99 - protein, 297 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPYIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 100 - protein, 681 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 101 - protein, 680 aa, artificial sequence
  synthetic polypeptide sequence
MALPVTALLLPLALLLHAARPYPYDVPDYAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 769 aa, artificial sequence
  synthetic polypeptide sequence
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGSADDAKKDAAKKDGKSMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 103 - protein, 767 aa, artificial sequence
  synthetic polypeptide sequence
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGGGGSGGGGSGGGGSMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 104 - protein, 548 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 105 - protein, 548 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 336 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 107 - protein, 301 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 108 - protein, 681 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 109 - protein, 680 aa, artificial sequence
  synthetic polypeptide sequence
MALPVTALLLPLALLLHAARPYPYDVPDYAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 548 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSSRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 111 - protein, 548 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSSRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 112 - protein, 333 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSS

SEQ ID NO: 113 - protein, 304 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 114 - protein, 681 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSSRKRRGKPIPNPLLGLDSTSGSGEGRGSLLTCGDVEENPGPMALPVTALLLPLALLLHAARPYPYDVPDYAMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 115 - protein, 680 aa, artificial sequence
  synthetic polypeptide sequence
MALPVTALLLPLALLLHAARPYPYDVPDYAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATNLSVIGFRILLLKVAGFNLLMTLRLWSSRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQILYEILLGKATLYAVLVSALVLMAMVKRKDF

SEQ ID NO: 116 - protein, 548 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSSRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 117 - protein, 548 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSSRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 118 - protein, 336 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSS

SEQ ID NO: 119 - protein, 301 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 120 - protein, 681 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSSRKRRGKPIPNPLLGLDSTSGSGEGRGSLLTCGDVEENPGPMALPVTALLLPLALLLHAARPYPYDVPDYAMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 121 - protein, 680 aa, artificial sequence
  synthetic polypeptide sequence
MALPVTALLLPLALLLHAARPYPYDVPDYAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCDVKLVEKSFETDTNLNFQNLSVIGFRILLLKVAGFNLLMTLRLWSSRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGFTSVSYQQGVLSATILYEILLGKATLYAVLVSALVLMAMVKRKDF

SEQ ID NO: 122 - protein, 791 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 123 - protein, 791 aa, artificial sequence
  synthetic polypeptide sequence
MALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 805 aa, artificial sequence
  synthetic polypeptide sequence
MALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASGGGGSGGGGSGGGGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 125 - protein, 805 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASGGGGSGGGGSGGGGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 126 - protein, 799 aa, artificial sequence
  synthetic polypeptide sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGGGGSGGGGSGGGGSSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 127 - protein, 799 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGGGGSGGGGSGGGGSSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 128 - protein, 664 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 129 - protein, 664 aa, artificial sequence
  synthetic polypeptide sequence
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 130 - protein, 658 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 131 - protein, 658 aa, artificial sequence
  synthetic polypeptide sequence
MALPVTALLLPLALLLHAARPEQKLISEEDLQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPYPYDVPDYAPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 132 - protein, 895 aa, artificial sequence
  synthetic polypeptide sequence
MALPVTALLLPLALLLHAARPEQKLISEEDLQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 133 - protein, 907 aa, artificial sequence
  synthetic polypeptide sequence
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSDP

SEQ ID NO: 134 - protein, 590 aa, artificial sequence
  synthetic polypeptide sequence
MALPVTALLLPLALLLHAARPYPYDVPDYAEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRKRRGKPIPNPLLGLDSTSGSGEGRGSLLTCGDVEENPGPMALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSKRGRKKLLYIFKQPFMRPVQTTQEEDGCSCRFPEEEEGGCEL

SEQ ID NO: 135 - protein, 19 aa, Artificial Sequence
  synthetic polypeptide sequence
RCRERRRNERLRRESVRPV

SEQ ID NO: 136 - protein, 29 aa, Artificial Sequence
  synthetic polypeptide sequence
RKGRMMDVKKCGIQDTNSKKQSDTHLEET

SEQ ID NO: 137 - protein, 32 aa, Artificial Sequence
  synthetic polypeptide sequence
KKRHMASYSMCSDPSTRDPPGRPEPYVEVYLI

SEQ ID NO: 138 - protein, 35 aa, Artificial Sequence
  synthetic polypeptide sequence
TKKKYSSSVHDPNGEYMFMRAVNTAKKSRLTDVTL

SEQ ID NO: 139 - protein, 35 aa, Artificial Sequence
  synthetic polypeptide sequence
MEESVVRPSVFVVDGQTDIPFTRLGRSHRRQSCSV

SEQ ID NO: 140 - protein, 39 aa, Artificial Sequence
  synthetic polypeptide sequence
SLSKMLKKRSPLTTGVYVKMPPTEPECEKQFQPYFIPIN

SEQ ID NO: 141 - protein, 40 aa, Artificial Sequence
  synthetic polypeptide sequence
ARTQIKKLCSWRDKNSAACVVYEDMSHSRCNTLSSPNQYQ

SEQ ID NO: 142 - protein, 42 aa, Artificial Sequence
  synthetic polypeptide sequence
KRGRKKLLYIFKQPFMRPVQTTQEEDGCSCRFPEEEEGGCEL

SEQ ID NO: 143 - protein, 43 aa, Artificial Sequence
  synthetic polypeptide sequence
WVRSKRSRLLHSDYMNMTPRRPGPTRKHYQPYAPPRDFAAYRS

SEQ ID NO: 144 - protein, 49 aa, Artificial Sequence
  synthetic polypeptide sequence
HQRRKYRSNKGESPVEPAEPCHYSCPREEEGSTIPIQEDYRKPEPACSP

SEQ ID NO: 145 - protein, 51 aa, Artificial Sequence
  synthetic polypeptide sequence
RRQWRPRRFSALEQGIHPPQAQSKIEELEQEPEPEPEPEPEPEPEPEPEQL

SEQ ID NO: 146 - protein, 54 aa, Artificial Sequence
  synthetic polypeptide sequence
HIWQLRSQCMWPRETQLLLEVPPSTEDARSCQFPEEERGERSAEEKGRLGDLWV

SEQ ID NO: 147 - protein, 61 aa, Artificial Sequence
  synthetic polypeptide sequence
NRRRRRERRDLFTESWDTQKAPNNYRSPISTSQPTNQSMDDTREDIYVNYPTFSRRPKTRV

SEQ ID NO: 148 - protein, 62 aa, Artificial Sequence
  synthetic polypeptide sequence
KKVAKKPTNKAPHPKQEPQEINFPDDLPGSNTAAPVQETLHGCQPVTQEDGKESRISVQERQ

SEQ ID NO: 149 - protein, 77 aa, Artificial Sequence
  synthetic polypeptide sequence
KWYSHSKEKIQNLSLISLANLPPSGLANAVAEGIRSEENIYTIEENVYEVEEPNEYYCYVSSRQQPSQPLGCRFAMP

SEQ ID NO: 150 - protein, 80 aa, Artificial Sequence
  synthetic polypeptide sequence
RKKKALRIHSVEGDLRRKSAGQEEWSPSAPSPPGSCVQAEAAPAGLCGEQRGEDCAELHDYFNVLSYRSLGNCSFFTETG

SEQ ID NO: 151 - protein, 83 aa, Artificial Sequence
  synthetic polypeptide sequence
KLRKAHVIWKKENEVSEHTLESYRSRSNNEETSSEEKNGQSSHPMRCMNYITKLYSEAKTKRKENVQHSKLEEKHIQVPESIV

SEQ ID NO: 152 - protein, 98 aa, Artificial Sequence
  synthetic polypeptide sequence
ICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 153 - protein, 101 aa, Artificial Sequence
  synthetic polypeptide sequence
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 154 - protein, 107 aa, Artificial Sequence
  synthetic polypeptide sequence
CCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 155 - protein, 114 aa, Artificial Sequence
  synthetic polypeptide sequence
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 156 - protein, 117 aa, Artificial Sequence
  synthetic polypeptide sequence
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 157 - protein, 120 aa, Artificial Sequence
  synthetic polypeptide sequence
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 158 - protein, 187 aa, Artificial Sequence
  synthetic polypeptide sequence
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 159 - protein, 108 aa, Artificial Sequence
  synthetic polypeptide sequence
TYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQ

SEQ ID NO: 160 - protein, 42 aa, Artificial Sequence
  synthetic polypeptide sequence
ALYLLRRDQRLPPDAHKPPGGGSFRTPIQEEQADAHSTLAKI

SEQ ID NO: 161 - protein, 51 aa, Artificial Sequence
  synthetic polypeptide sequence
MGWIRGRRSRHSWEMSEFHNYNLDLKKSDFSTRWQKQRCPVVKSKCRENAS

SEQ ID NO: 162 - protein, 48 aa, Artificial Sequence
  synthetic polypeptide sequence
KKYFFKKEVQQLSVSFSSLQIKALQNAVEKEVQAEDNIYIENSLYATD

SEQ ID NO: 163 - protein, 22 aa, Artificial Sequence
  synthethic polypeptide
SGFLQEKVWVMLVTSLVALQAL

SEQ ID NO: 164 - protein, 85 aa, Artificial Sequence
  synthetic polypeptide sequence
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 165 - protein, 111 aa, Artificial Sequence
  synthethic polypeptide
ACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 166 - protein, 95 aa, Artificial Sequence
  synthetic polypeptide sequence
MAEAITYADLRFVKAPLKKSISSRLGQDPGADDDGEITYENVQVPAVLGVPSSLASSVLGDKAAVKSEQPTASWRAVTSPAVGRILPCRTTCLRY

SEQ ID NO: 167 - protein, 141 aa, Artificial Sequence
  synthetic polypeptide sequence
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 168 - protein, 45 aa, Artificial Sequence
  synthethic polypeptide
RKWCQYQKEIMERPPPFKPPPPPIKYTCIQEPNESDLPYHEMETL

SEQ ID NO: 169 - protein, 84 aa, Artificial Sequence
  synthetic polypeptide sequence
LRKRRDSLSLSTQRTQGPAESARNLEYVSVSPTNNTVYASVTHSNRETEIWTPRENDTITIYSTINHSKESKPTFSRATALDNV

SEQ ID NO: 170 - protein, 88 aa, Artificial Sequence
  synthetic polypeptide sequence
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 171 - protein, 82 aa, Artificial Sequence
  synthetic polypeptide sequence
KTHRRKAARTAVGRNDTHPTTGSASPKHQKKSKLHGPTETSSCSGAAPTVEMDEELHYASLNFHGMNPSKDTSTEYSEVRTQ

SEQ ID NO: 172 - protein, 38 aa, Artificial Sequence
  synthethic polypeptide
MTDSVIYSMLELPTATQAQNDYGPQQKSSSSRPSCSCL

SEQ ID NO: 173 - protein, 45 aa, Artificial Sequence
  synthethic polypeptide
MDQQAIYAELNLPTDSGPESSSPSSLPRDVCQGSPWHQFALKLSC

SEQ ID NO: 174 - protein, 168 aa, Artificial Sequence
  synthetic polypeptide sequence
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 175 - protein, 83 aa, Artificial Sequence
  synthetic polypeptide sequence
RWCSNKKNAAVMDQESAGNRTANSEDSDEQDPQEVTYTQLNHCVFTQRKITRPSQRPKTPPTDIIVYTELPNAESRSKVVSCP

SEQ ID NO: 176 - protein, 84 aa, Artificial Sequence
  synthetic polypeptide sequence
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 177 - protein, 10 aa, Artificial Sequence
  synthethic polypeptide
MAVFKTTLWR

SEQ ID NO: 178 - protein, 29 aa, Artificial Sequence
  synthethic polypeptide
KGSQRVPEEPGEQPIYMNFSEPLTKDMAT

SEQ ID NO: 179 - protein, 11 aa, Artificial Sequence
  synthethic polypeptide
VNRPQWAPPGR

SEQ ID NO: 180 - protein, 8 aa, Artificial Sequence
  synthethic polypeptide
VTLRSFVP

SEQ ID NO: 181 - protein, 35 aa, Artificial Sequence
  synthethic polypeptide
AAWHGQKPGTHPPSELDCGHDPGYQLQTLPGLRDT

SEQ ID NO: 182 - protein, 32 aa, Artificial Sequence
  synthethic polypeptide
QHSQRSPPRCSQEANSRKDNAPFRVVEPWEQI

SEQ ID NO: 183 - protein, 168 aa, Artificial Sequence
  synthetic polypeptide sequence
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 184 - protein, 116 aa, Artificial Sequence
  synthetic polypeptide sequence
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 185 - protein, 126 aa, Artificial Sequence
  synthetic polypeptide sequence
MAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTP

SEQ ID NO: 186 - protein, 61 aa, Artificial Sequence
  synthetic polypeptide sequence
KVNGCRKYKLNKTESTPVVEEDEMQPYASYTEKNNPLYDTTNKVKASEALQSEVDTDLHTL

SEQ ID NO: 187 - protein, 98 aa, Artificial Sequence
  synthetic polypeptide sequence
RMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 188 - protein, 113 aa, Artificial Sequence
  synthetic polypeptide sequence
WRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 189 - protein, 37 aa, Artificial Sequence
  synthetic polypeptide sequence
MSDSKEPRLQQLGLLEEEQLRGLGFRQTRGYKSLAGC

SEQ ID NO: 190 - protein, 29 aa, Artificial Sequence
  synthetic polypeptide sequence
RKSSGGKGGSYSQAACSDSAQGSDVSLTA

SEQ ID NO: 191 - protein, 95 aa, Artificial Sequence
  synthetic polypeptide sequence
LPKYKTRKAMRNNVPRDRGDTAMEVGIYANILEKQAKEESVPEVGSRPCVSTAQDEAKHSQELQYATPVFQEVAPREQEACDSYKSGYVYSELNF

SEQ ID NO: 192 - protein, 178 aa, Artificial Sequence
  synthethic polypeptide
RRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDMEQ

SEQ ID NO: 193 - protein, 42 aa, Artificial Sequence
  synthethic polypeptide
SAGSAGSAGSAGSAGSAGSAGSAGSAGSAGSAGSAGSAGSAG

SEQ ID NO: 194 - protein, 42 aa, Artificial Sequence; identical to SEQ ID NO: 142
  synthethic polypeptide

SEQ ID NO: 195 - protein, 93 aa, Artificial Sequence
  synthetic polypeptide sequence
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 196 - protein, 118 aa, Artificial Sequence
  synthetic polypeptide sequence
KCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 197 - protein, 74 aa, Artificial Sequence
  synthetic polypeptide sequence
HFGKTGRASDQRDLTEHKPSVSNHTQDHSNDPPNKMNEVTYSTLNFEAQQPTQPTSASPSLTATEIIYSEVKKQ

SEQ ID NO: 198 - protein, 112 aa, Artificial Sequence
  synthetic polypeptide sequence
DVVCTGWLRKSPPEKKLRRYAWKKRWFILRSGRMSGDPDVLEYYKNDHSKKPLRIINLNFCEQVDAGLTFNKKELQDSFVFDIKTSERTFYLVAETEEDMNKWVQSICQICG

SEQ ID NO: 199 - protein, 116 aa, Artificial Sequence
  synthetic polypeptide sequence
AVMEGPLFLQSQRFGTKRWRKTWAVLYPASPHGVARLEFFDHKGSSSGGGRGSSRRLDCKVIRLAECVSVAPVTVETPPEPGATAFRLDTAQRSHLLAADAPSSAAWVQTLCRNAF

SEQ ID NO: 200 - protein, 111 aa, Artificial Sequence
  synthetic polypeptide sequence
GAVKQGFLYLQQQQTFGKKWRRFGASLYGGSDCALARLELQEGPEKPRRCEAARKVIRLSDCLRVAEAGGEASSPRDTSAFFLETKERLYLLAAPAAERGDWVQAICLLAF

SEQ ID NO: 201 - DNA, 2378 bp, Artificial Sequence
  synthetic polynucleotide
aggcaggtggacagtggatcgccgccgagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggttctgcggatgatgcgaagaaagatgcggcaaagaaagatggtaagagcatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcg

SEQ ID NO: 202 - DNA, 2372 bp, Artificial Sequence
  synthetic polynucleotide
aggcaggtggacagtggatcgccgccgagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggtggcggtggctcgggcggtggtgggtcgggtggcggcggatctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcg

SEQ ID NO: 203 - DNA, 1690 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 204 - DNA, 1690 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 205 - DNA, 1666 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaagacttgagaaatgtaactccccccaaggtaagcctctttgaacctagcaaggcggaaattgcgaacaagcaaaaagcgaccctggtatgcctcgctcggggctttttcccagatcacgttgagctgagctggtgggtcaacggaaaggaagttcacagcggggttagcaccgacccccaagcttataaggaatcaaattattcatattgtctctcatcaagattgcgcgtatgtgcaactttttggcataacccaagaaaccattttcgctgccaggttcagttccacggcctgagtgaggaagacaaatggcccgaggggagtcccaaacccgtcacgcaaaatataagcgcggaggcatggggtagagctgattgcggcattacctccgcatcatatcagcagggggtgctgagcgcaacaatcctttatgaaatactccttggaaaggccacactttacgccgtactcgtcagcacccttgtcgtaatggctatggtcaaacggaagaatagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctccctacatccaaaaccctgaacctgctgtatatcagctgaaagatccacgcagtcaggattcaacgctgtgtctttttaccgactttgacagtcagatcaatgtccccaaaactatggagtcaggcaccttcataacggataagacggttctggacatgaaagccatggattctaaatcaaacggagcaatcgcctggagcaaccagactagcttcacctgccaggatatattcaaagaaaccaatgcttgttatccttcttctgatgtaccctgtgatgcgaccctgactgaaaaatcctttgagacggacatgaatctgaattttcaaaatctttctgtaatgggactgaggatcttgcttttgaaagttgctggctttaaccttctgatgacgctgcgcctgtggagtagcgatccttgacttgcggccgc

SEQ ID NO: 206 - DNA, 1666 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaagacttgagaaatgtaactccccccaaggtaagcctctttgaacctagcaaggcggaaattgcgaacaagcaaaaagcgaccctggtatgcctcgctcggggctttttcccagatcacgttgagctgagctggtgggtcaacggaaaggaagttcacagcggggttagcaccgacccccaagcttataaggaatcaaattattcatattgtctctcatcaagattgcgcgtatgtgcaactttttggcataacccaagaaaccattttcgctgccaggttcagttccacggcctgagtgaggaagacaaatggcccgaggggagtcccaaacccgtcacgcaaaatataagcgcggaggcatggggtagagctgattgcggcattacctccgcatcatatcagcagggggtgctgagcgcaacaatcctttatgaaatactccttggaaaggccacactttacgccgtactcgtcagcacccttgtcgtaatggctatggtcaaacggaagaatagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtccctacatccaaaaccctgaacctgctgtatatcagctgaaagatccacgcagtcaggattcaacgctgtgtctttttaccgactttgacagtcagatcaatgtccccaaaactatggagtcaggcaccttcataacggataagacggttctggacatgaaagccatggattctaaatcaaacggagcaatcgcctggagcaaccagactagcttcacctgccaggatatattcaaagaaaccaatgcttgttatccttcttctgatgtaccctgtgatgcgaccctgactgaaaaatcctttgagacggacatgaatctgaattttcaaaatctttctgtaatgggactgaggatcttgcttttgaaagttgctggctttaaccttctgatgacgctgcgcctgtggagtagcgatccttgacttgcggccgc

SEQ ID NO: 207 - DNA, 1054 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 208 - DNA, 949 bp, Artificial Sequence
  synthetic polynucleotide
aggcaggtggacagtggatcgccgccatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 209 - DNA, 1042 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaagacttgagaaatgtaactccccccaaggtaagcctctttgaacctagcaaggcggaaattgcgaacaagcaaaaagcgaccctggtatgcctcgctcggggctttttcccagatcacgttgagctgagctggtgggtcaacggaaaggaagttcacagcggggttagcaccgacccccaagcttataaggaatcaaattattcatattgtctctcatcaagattgcgcgtatgtgcaactttttggcataacccaagaaaccattttcgctgccaggttcagttccacggcctgagtgaggaagacaaatggcccgaggggagtcccaaacccgtcacgcaaaatataagcgcggaggcatggggtagagctgattgcggcattacctccgcatcatatcagcagggggtgctgagcgcaacaatcctttatgaaatactccttggaaaggccacactttacgccgtactcgtcagcacccttgtcgtaatggctatggtcaaacggaagaatagcgatccttgacttgcggccgc

SEQ ID NO: 210 - DNA, 937 bp, Artificial Sequence
  synthetic polynucleotide
aggcaggtggacagtggatcgccgccatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtccctacatccaaaaccctgaacctgctgtatatcagctgaaagatccacgcagtcaggattcaacgctgtgtctttttaccgactttgacagtcagatcaatgtccccaaaactatggagtcaggcaccttcataacggataagacggttctggacatgaaagccatggattctaaatcaaacggagcaatcgcctggagcaaccagactagcttcacctgccaggatatattcaaagaaaccaatgcttgttatccttcttctgatgtaccctgtgatgcgaccctgactgaaaaatcctttgagacggacatgaatctgaattttcaaaatctttctgtaatgggactgaggatcttgcttttgaaagttgctggctttaaccttctgatgacgctgcgcctgtggagtagcgatccttgacttgcggccgc

SEQ ID NO: 211 - DNA, 2089 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 212 - DNA, 2086 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 213 - DNA, 2383 bp, Artificial Sequence
  synthetic polynucleotide
aggcaggtggacagtggatcgccgccgagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggttctgcggatgatgcgaagaaagatgcggcaaagaaagatggtaagagcatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 214 - DNA, 2377 bp, Artificial Sequence
  synthetic polynucleotide
aggcaggtggacagtggatcgccgccgagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggtggcggtggctcgggcggtggtgggtcgggtggcggcggatctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 215 - DNA, 1690 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 216 - DNA, 1690 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 217 - DNA, 1054 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 218 - DNA, 949 bp, Artificial Sequence
  synthetic polynucleotide
aggcaggtggacagtggatcgccgccatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 219 - DNA, 2089 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 220 - DNA, 2086 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 221 - DNA, 1690 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaatactgtatgagattcttctcgggaaagcgactctttacgccgtgctcgtatccgccttggtactgatggctatggtgaaaagaaaggactttgatccttgacttgcggccgc

SEQ ID NO: 222 - DNA, 1690 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaatactgtatgagattcttctcgggaaagcgactctttacgccgtgctcgtatccgccttggtactgatggctatggtgaaaagaaaggactttgatccttgacttgcggccgc

SEQ ID NO: 223 - DNA, 1045 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactaatctctccgtgattgggtttagaatcttgctgctgaaagtggccggattcaatctgcttatgacccttcggctctggtccagcgatccttgacttgcggccgc

SEQ ID NO: 224 - DNA, 958 bp, Artificial Sequence
  synthetic polynucleotide
aggcaggtggacagtggatcgccgccatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaatactgtatgagattcttctcgggaaagcgactctttacgccgtgctcgtatccgccttggtactgatggctatggtgaaaagaaaggactttgatccttgacttgcggccgc

SEQ ID NO: 225 - DNA, 2089 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaatactgtatgagattcttctcgggaaagcgactctttacgccgtgctcgtatccgccttggtactgatggctatggtgaaaagaaaggactttgatccttgacttgcggccgc

SEQ ID NO: 226 - DNA, 2086 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaatactgtatgagattcttctcgggaaagcgactctttacgccgtgctcgtatccgccttggtactgatggctatggtgaaaagaaaggactttgatccttgacttgcggccgc

SEQ ID NO: 227 - DNA, 1690 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgacgttaaactggtggagaagagctttgaaaccgacacaaatctgaacttccaaaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 228 - DNA, 1690 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgacgttaaactggtggagaagagctttgaaaccgacacaaatctgaacttccaaaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 229 - DNA, 1054 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 230 - DNA, 949 bp, Artificial Sequence
  synthetic polynucleotide
aggcaggtggacagtggatcgccgccatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 231 - DNA, 2089 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgacgttaaactggtggagaagagctttgaaaccgacacaaatctgaacttccaaaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctatggagactctcctgggcctcctgattctgtggcttcagttgcaatgggtttcctctaagcaagaagtgacccagattcctgccgccctgagtgtgccagaaggcgaaaacctggtgctcaactgctcatttacggatagcgcaatttacaacctccagtggtttagacaggacccaggtaaaggactcacgagcttgcttctgatccaaagctcacagagagagcagacctctgggaggcttaacgcgtcccttgataagtcatctgggcgctctacgctgtacatcgcggctagccaaccgggggattctgccacatacctgtgcgcagtgagaccaacaagtgggggctcttatatccctaccttcgggagagggaccagcctgatcgttcatccgcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 232 - DNA, 2086 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctatgtccatcggcctgctgtgctgcgccgcactgtccctgctgtgggcaggtcctgtcaatgccggagtcacccagacgccaaagttccaggtactgaaaaccgggcagtccatgacgttgcagtgtgcacaggacatgaaccatgaatatatgagctggtataggcaggatcctggaatgggactgagactcatacactatagtgtgggggcaggaatcacagatcaaggtgaagtcccgaacgggtacaatgtctcccggtccaccaccgaggacttcccactgcgacttctcagtgcagctcccagtcagacctccgtgtacttctgtgcaagttcttatgtcggtaatacaggagaactgttttttggggaaggctcccgactgacagttctggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtcaaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgacgttaaactggtggagaagagctttgaaaccgacacaaatctgaacttccaaaatctctccgtgattgggtttagaatcttgctgctgaaagtggccggattcaatctgcttatgacccttcggctctggtccagcaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaaaccgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttgatccttgacttgcggccgc

SEQ ID NO: 233 - DNA, 2419 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccttaccagtgaccgccttgctcctgccgctggccttgctgctccacgccgccaggccggattacaaggatgacgatgacaagggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagccccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 234 - DNA, 2419 bp, Artificial Sequence
  synthetic polynucleotide
aggcaggtggacagtggatcgccgccatggccttaccagtgaccgccttgctcctgccgctggccttgctgctccacgccgccaggccggattacaaggatgacgatgacaagggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagcgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 235 - DNA, 2461 bp, Artificial Sequence
  synthetic polynucleotide
aggcaggtggacagtggatcgccgccatggccttaccagtgaccgccttgctcctgccgctggccttgctgctccacgccgccaggccggattacaaggatgacgatgacaagggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagcggtggcggtggctcgggcggtggtgggtcgggtggcggcggatctatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 236 - DNA, 2461 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccttaccagtgaccgccttgctcctgccgctggccttgctgctccacgccgccaggccggattacaaggatgacgatgacaagggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagcggtggcggtggctcgggcggtggtgggtcgggtggcggcggatctatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 237 - DNA, 2443 bp, Artificial Sequence
  synthetic polynucleotide
aggcaggtggacagtggatcgccgccatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggtggcggtggctcgggcggtggtgggtcgggtggcggcggatcttctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagcgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 238 - DNA, 2443 bp, Artificial Sequence
  synthetic polynucleotide
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccttaccagtgaccgccttgctcctgccgctggccttgctgctccacgccgccaggccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtggtggcggtggctcgggcggtggtgggtcgggtggcggcggatcttctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagccccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgc

SEQ ID NO: 239 - DNA, 2033 bp, Artificial Sequence
  synthetic polynucleotide sequence
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccctgccagtcacagctctgttgttgcccctggctctgttgctccatgccgcgcgacccgagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcg

SEQ ID NO: 240 - DNA, 2033 bp, artificial sequence
  synthetic polynucleotide sequence
aggcaggtggacagtggatcgccgccatggccctgccagtcacagctctgttgttgcccctggctctgttgctccatgccgcgcgacccgagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcg

SEQ ID NO: 241 - DNA, 2015 bp, artificial sequence
  synthetic polynucleotide sequence
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccttaccagtgaccgccttgctcctgccgctggccttgctgctccacgccgccaggccggagcagaagctgatcagcgaggaggacctgcaggtccaactgcagcaatctggaccaggacttgtaaaaccaagtcaaactctgagcttgacctgtgcaataagcggtgacagtgtgagtagcaattctgccgcgtggaattggatcagacaaagtccaagtcgaggtttggagtggcttggcaggacttattaccgctcaaagtggtataatgactacgcagtgagcgtcaagtcaaggattacgataaacccggacacctctaaaaaccagttttccctccaacttaactcagtgaccccggaggatacagctgtttactactgtgcgcgggaggttacaggagacctggaagatgcgttcgacatctggggacagggcacgatggtgactgtttcctctggaggtggcggatcagacatacagatgacccaatcaccctcaagtttgtcagcaagtgtcggtgatagggtaaccattacatgcagggcaagccaaaccatctggtcttacttgaattggtaccagcagagaccggggaaggctcccaacctccttatatatgcagcatcaagcctccagagtggagtacccagccgatttagtgggcgggggagtggtaccgactttacactgaccatctccagcctccaggccgaggacttcgctacttattattgccaacaatcatactccataccccaaacctttgggcaagggaccaaattggagatcaagcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcg

SEQ ID NO: 242 - DNA, 2015 bp, artificial sequence
  synthetic polynucleotide sequence
aggcaggtggacagtggatcgccgccatggccttaccagtgaccgccttgctcctgccgctggccttgctgctccacgccgccaggccggagcagaagctgatcagcgaggaggacctgcaggtccaactgcagcaatctggaccaggacttgtaaaaccaagtcaaactctgagcttgacctgtgcaataagcggtgacagtgtgagtagcaattctgccgcgtggaattggatcagacaaagtccaagtcgaggtttggagtggcttggcaggacttattaccgctcaaagtggtataatgactacgcagtgagcgtcaagtcaaggattacgataaacccggacacctctaaaaaccagttttccctccaacttaactcagtgaccccggaggatacagctgtttactactgtgcgcgggaggttacaggagacctggaagatgcgttcgacatctggggacagggcacgatggtgactgtttcctctggaggtggcggatcagacatacagatgacccaatcaccctcaagtttgtcagcaagtgtcggtgatagggtaaccattacatgcagggcaagccaaaccatctggtcttacttgaattggtaccagcagagaccggggaaggctcccaacctccttatatatgcagcatcaagcctccagagtggagtacccagccgatttagtgggcgggggagtggtaccgactttacactgaccatctccagcctccaggccgaggacttcgctacttattattgccaacaatcatactccataccccaaacctttgggcaagggaccaaattggagatcaaggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccgtacccatacgatgttccagattacgctcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcg

SEQ ID NO: 243 - DNA, 2726 bp, artificial sequence
  synthetic polynucleotide sequence
aggcaggtggacagtggatcgccgccatggccctgcctgttaccgcgcttcttctcccactcgcgctgctcctccatgctgcacgccccgagcagaaattgatttctgaggaagaccttcaggttcagctccagcagagtgggcccggcctggttaaaccatctcagactctttctcttacttgcgccatttctggggactcagtgtcatcaaactcagcagcatggaattggatcaggcaaagtccttctagaggacttgagtggctgggtagaacctactaccgctctaaatggtacaatgactatgcagtcagtgtaaaaagccgaataacgattaaccccgatacgtcaaaaaaccaattctctcttcaattgaactctgtcacgcccgaagacacggctgtttactattgcgcccgggaggtaaccggagacttggaagacgcttttgatatctggggtcaggggactatggtaacggtttcttccgggggcgggggttcagatattcagatgacacaatcaccttcctccttgagcgcctccgtgggtgacagggtcaccataacgtgtcgagctagccaaactatttggtcctaccttaactggtatcaacagcgacccggcaaggcccctaacttgcttatctacgctgcttcatccttgcaatcaggtgtgccgtcccgcttcagtggtcgaggctccggtacggactttacgctcacaataagttcactccaagcagaggatttcgccacctattactgccagcaaagctattctattcctcaaaccttcggacaggggaccaagctggagattaaggaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccttaccagtgaccgccttgctcctgccgctggccttgctgctccacgccgccaggccggagcagaagctgatcagcgaggaggacctgcaggtccaactgcagcaatctggaccaggacttgtaaaaccaagtcaaactctgagcttgacctgtgcaataagcggtgacagtgtgagtagcaattctgccgcgtggaattggatcagacaaagtccaagtcgaggtttggagtggcttggcaggacttattaccgctcaaagtggtataatgactacgcagtgagcgtcaagtcaaggattacgataaacccggacacctctaaaaaccagttttccctccaacttaactcagtgaccccggaggatacagctgtttactactgtgcgcgggaggttacaggagacctggaagatgcgttcgacatctggggacagggcacgatggtgactgtttcctctggaggtggcggatcagacatacagatgacccaatcaccctcaagtttgtcagcaagtgtcggtgatagggtaaccattacatgcagggcaagccaaaccatctggtcttacttgaattggtaccagcagagaccggggaaggctcccaacctccttatatatgcagcatcaagcctccagagtggagtacccagccgatttagtgggcgggggagtggtaccgactttacactgaccatctccagcctccaggccgaggacttcgctacttattattgccaacaatcatactccataccccaaacctttgggcaagggaccaaattggagatcaagcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcg

SEQ ID NO: 244 - DNA, 2782 bp, artificial sequence
  synthetic polynucleotide sequence
aagtggagcaaggcaggtggacagtggatcgccgccatggcactgccggtcacagctctccttctgcctctcgcattgctcctccacgcagcgagaccagagcaaaaattgatctccgaagaagaccttgatatacaaatgacacagaccacctcttcattgtccgctagccttggggaccgggtaaccatttcctgccgggcgtcacaggacatcagcaaatacttgaactggtatcaacagaaacccgacgggacagtcaaacttttgatatatcatacctcccgactgcactctggggttccttcaagatttagtggaagcggctctggcactgactacagtttgactatcagtaatcttgaacaggaggacattgctacctacttttgtcaacaaggtaacacgctgccgtacactttcggcgggggaacaaagctcgaaatcactggcggtggagggtcaggtggagggggttctgggggcggtgggagtgaagttaaacttcaagaatcaggtcctggtttggtagcaccaagccagtcactttctgtcacttgcaccgtgagcggagtcagcttgccggattacggcgtctcatggatacgccagccccctagaaagggtctggagtggctgggtgtcatatggggatccgagactacatactataacagcgcactgaagagtaggttgacaataattaaggataattctaaatctcaggtctttctgaaaatgaactccctgcaaaccgatgacaccgccatttactattgcgcaaagcattactattacggaggatcttacgccatggattactgggggcaggggacttcagttactgtatcttcagaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggccctgccagtcacagctctgttgttgcccctggctctgttgctccatgccgcgcgacccgagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcgatccttgacttgcggccgcaactcccaccg

SEQ ID NO: 245 - DNA, 1819 bp, artificial sequence
  synthetic polynucleotide sequence
aggcaggtggacagtggatcgccgccatggccttaccagtgaccgccttgctcctgccgctggccttgctgctccacgccgccaggccgtacccatacgatgttccagattacgctgaggaccttaataaagtgtttccacctgaggtggccgtgtttgagcctagtgaggcagaaatcagtcatacccagaaggccacactcgtgtgtctcgccacagggttttttccggaccatgttgaactttcttggtgggtgaacggcaaagaggtgcactctggcgtgtgtaccgatccccagcccttgaaggaacagcctgccctgaacgacagccgctactgcctgagctcccgcctgagggtgagtgccacattttggcagaatccacggaatcatttcagatgccaggtacagttctacggcttgtcagagaatgatgagtggacccaggaccgcgcgaaacccgtcacacagattgtcagtgccgaggcctgggggagagctgactgcgggttcaccagtgtgtcctatcaacagggcgtgctgtcagccactatactgtatgagattcttctcgggaaagcgactctttacgccgtgctcgtatccgccttggtactgatggctatggtgaaaagaaaggactttaggaagcgacggggaaaacctatccctaatccactgcttggtctcgatagtactagtgggtcaggcgaggggagaggctcactcctcacgtgtggagacgttgaagaaaaccccggaccaatggcgcttccagtaactgccttgttgctgccactggccctgctcctccatgctgcaagaccggagcagaagctgatcagcgaggaggacctgatggcagacgtccagcttgtagagtcaggaggggggctcgtgcaggccggcggcagccttcgcctgtcctgcgccgcctcagggagaaccataagcatggctgccatgtcttggtttcgccaggcccctggtaaagagagagaattcgtagccggcataagccgcagtgctggttccgccgttcacgcagattccgtgaaaggcagattcacaatttcacgggacaacacgaagaataccctgtaccttcagatgaacagtctcaaggccgaagacaccgcggtgtattactgtgctgtgagaacgagcgggttctttggaagcatcccccgaactggcaccgccttcgactactggggccaaggcacacaagtgactgttagtcccaacatccagaatcctgatcctgccgtctatcagcttcgcgacagtaaaagtagtgataaatcagtgtgtctttttacagacttcgattcccagaccaacgtgagtcagtccaaggattccgacgtgtatattaccgacaaatgtgtcctcgacatgaggagcatggatttcaaatcaaactcagctgtggcctggtccaataagtctgatttcgcttgtgccaatgcattcaataatagcatcattcctgaggatactttcttcccatctccagagtcaagctgcgacgttaaactggtggagaagagctttgaaaccgacacaaatctgaacttccaaaatctctccgtgattgggtttagaatcttgctgctgaaagtggccggattcaatctgcttatgacccttcggctctggtccagcaaacgcgggcgaaagaagcttctttatatttttaagcagccatttatgcgaccggtacaaacaacacaagaggaagacgggtgtagctgcaggtttcctgaagaagaggagggaggttgtgagctgtgagatccttgacttgcggccgc